(19) United States
(12) Patent Application Publication
(10) Pub. No.: US 2025/0346587 A1
(43) Pub. Date: Nov. 13, 2025

(54) NITROGEN-CONTAINING FIVE-MEMBERED HETEROCYCLIC DERIVATIVES AS CHECKPOINT KINASE 1 INHIBITOR AND USES THEREOF

(71) Applicant: SPEROGENIX THERAPEUTICS LIMITED, Hong Kong (CN)

(72) Inventors: Hanlan LIU, Hong Kong (CN); Jingjing MA, Hong Kong (CN); Yung-Jen HUANG, Hong Kong (CN); Zhang CAI, Hong Kong (CN); Xiaohui WANG, Hong Kong (CN); Zhiyu YAN, Hong Kong (CN); Pasha M. KHAN, New Delhi (IN); Dayanand PANPATIL, Noida (IN); Brahmam PUJALA, Greater Noida (IN); Juxin RUAN, Hong Kong (CN)

(21) Appl. No.: 18/869,156

(22) PCT Filed: Apr. 23, 2023
(86) PCT No.: PCT/CN2023/090103

§ 371 Date: Nov. 25, 2024

(30) Foreign Application Priority Data
May 25, 2022 (WO) PCT/CN2022/095077

(51) Int. Cl. C07D 413/14; A61K 31/497; A61K 31/5377; C07D 401/14; C07D 403/12; C07D 403/14; C07D 413/12

(52) U.S. Cl. CPC C07D 413/14; A61K 31/497; A61K 31/5377; C07D 401/14; C07D 403/12; C07D 403/14; C07D 413/12

(57) ABSTRACT

The present disclosure relates generally to nitrogen-containing five-membered heterocyclic derivatives usefulness in treatment of conditions associated with Checkpoint kinase (CHK), particularly more specifically CHK-1 enzymes. Methods of use, compositions, and method of synthesis are also disclosed.

### FIELD OF THE INVENTION

The present invention generally relates to nitrogen-containing five-membered heterocyclic derivatives having Checkpoint kinase-1 (CHK-1) inhibitory activity, to the use of such compounds in the treatment of proliferative disorders, such as cancer; Pulmonary Arterial Hypertension (PAH) and Idiopathic Pulmonary Fibrosis (IPF). The invention also provides method of synthesis of said compounds, method of using said compounds, pharmaceutical compositions comprising said compounds and method of using thereof.

### BACKGROUND OF THE INVENTION

A wide range of cancer chemotherapeutic agents act through DNA damaging pathway to induce DNA damage causing tumor growth inhibition. However, these chemotherapeutic agents lead to cell cycle arrest by induction of checkpoints at either S-phase or G2/M boundary. The G2 arrest allows the cell time to repair the damaged DNA before entering mitosis. Checkpoint kinase-1 (CHK-1) and an unrelated serine/threonine kinase, Checkpoint kinase-2 (CHK-2), play a central role in arresting the cell cycle at the G2-M boundary (O'Connell et al., 1997). CHK-1 and/or CHK-2 induce this checkpoint by phosphorylating CDC25 phosphatase, inhibiting the removal of inactivating phosphates on cyclin dependent kinases (CDKs) (Karlsson-Rosenthal et al., 2006; Zheng et al., 1998). Another overlapping pathway mediated by p53 also elicits cycle arrest in response to DNA-damage. However, p53 is mutationally inactivated in many cancers, resulting in a partial deficiency in their ability to initiate a DNA-repair response. If CHK-1 activity is also inhibited in p53-negative cancers, all ability to arrest and repair DNA in response to DNA-damage will be removed, and this results in mitotic catastrophe and enhances the effect of the DNA damaging agents (Bunch & Eastman, 1996; Konarias et al. 2001; Tenzer & Pruschy 2003).

CHK-1 inhibition, therefore, represents a novel therapeutic strategy to increase the lethality of DNA-damaging chemotherapeutic drugs in p53 pathway defective cancers (Ma et al., 2012). Abrogation of the remaining intact checkpoint should result in increased tumor cell death. CHK-1 inhibitors have demonstrated potentiation of a range of cytotoxic chemotherapy drugs both in vitro and in a range of pre-clinical models of human cancer including gemcitabine, irinotecan, cytarabine, and cisplatin (Qiu et al., 2018). This “synthetic lethality” approach should increase the therapeutic activity of the chemotherapeutic drug without increasing the systemic toxicity as normal cells should remain protected by their functional p53 pathway. CHK-1 inhibitors have, therefore, the potential to be combined with a wide range of cytotoxic chemotherapeutic agents for the treatment of a diverse selection of human cancers.

Further, excessive and sustained proliferation, resistance to apoptosis by fine tuning of cell cycle, and DNA repair machinery are few of the causative mechanisms of cancer, Pulmonary Arterial Hypertension (PAH), and Idiopathic Pulmonary Fibrosis (IPF). PAH is a devastating disease accompanied with progressive vascular remodeling of distal pulmonary arteries leading to concomitant elevation of pulmonary artery pressure, perivascular inflammation, fibrotic changes, right ventricular hypertrophy, and death (Bourgeois et al., 2019). It is a concurrent complication of Idiopathic Pulmonary Fibrosis and affects its survival, functional status, and progression, however, no treatment other than lung transplantation are currently available. Besides genetic predisposition, a number of epigenetic factors such as oxidative stress and generation of reactive oxygen species cause DNA damage in pulmonary artery smooth muscle cells (PASMCs) and alter the cellular functions similar to cancer cells (Ranchoux et al., 2016). Since DNA repair machinery has been targeted successfully to delineate the underlying molecular mechanisms of cancer and a wide range of chemotherapeutic agents are being explored, which act through DNA damaging pathway to cause tumor growth inhibition, similar approach could be adopted for PAH and IPF (Sharma & Aldred, 2020; Wu et al., 2022). In cancer, these chemotherapeutic agents lead to cell cycle arrest by induction of checkpoints at either S-phase or G2/M boundary, wherein, the G2 arrest allows the cell to repair the damaged DNA before entering mitosis. Checkpoint kinase-1 (CHK-1) and Checkpoint kinase-2 (CHK-2), serine/threonine kinases, are key components of the DNA damage response and critical regulators of DNA repair and cell cycle progression. They are upregulated in cancer cells and play a central role in arresting the cell cycle at the G2-M boundary to facilitate DNA repair (O'Connell et al., 1997). CHK-1 and/or CHK-2 induce this checkpoint by phosphorylating CDC25 phosphatase, inhibiting the removal of inactivating phosphates on cyclin dependent kinases (CDKs) (Zheng et al., 1998). In PAH, proliferating PAH-PASMCs show increased levels of γ-H2AX and pRPA32, markers for DNA damage/replication stress and also display enhanced expression and activation of CHK1. Moreover, pharmacological inhibition of CHK1 reduces vascular remodeling and improves hemodynamic parameters in clinically relevant rat models suggesting that CHK1 inhibition could also be an attractive therapeutic option for PAH (Bourgeois et al., 2019).

Various attempts have been made to develop CHK-1 kinase inhibitors. For example, US10000481B2 (Vernalis) disclosed 1H-pyrrolo[2,3-B] pyridine derivatives compounds as CHK-1 kinase inhibitors. U.S. Pat. No. 10,010,547B2 (Cascadian Therapeutics) disclosed pyrazol amino pyrazine derivatives as kinase inhibitors. WO/2018/086546A1 (Zhejiang University) disclosed 2-polysubstituted aromatic ring-pyrimidine derivatives as CHK-1 inhibitors. Some small molecule inhibitors of CHK-1 (Prexasertib/LY2606368, LY2603618 and SRA737) are currently in Phase I/II clinical evaluation in combination with gemcitabine, pemetrexed, fludarabine, cytarabine, and cisplatin. These CHK-1 kinase inhibitors are not nitrogen-containing five-membered heterocyclic derivatives. The main features of pulmonary hypertension (PH) in IPF patients (IPF-PH) are excessive proliferation and resistance to apoptosis of fibroblasts and pulmonary arterial (PA) smooth muscle cells (PASMC) that lead to aberrant accumulation of extracellular matrix in parenchyma and extensive vascular remodeling. It can be hypothesized that CHK1/2, which is upregulated and activated, contributes to fibrotic and vascular lesions in IPF-PH. This is associated with γH2AX, a sensitive molecular marker of DNA damage, which in turn correlates with PAH remodeling and fibrosis scores (Sharma & Aldred, 2020). The increase in DNA repair in IPF is associated with a significant upregulation of CHK1 and CHK2 in the lungs and distal PA of IPF patients, and it was mainly localized within PASMC and fibrotic lesions. Some of the drugs targeting proliferation could be protective against PAH and since CHK1 activation in PAH-PASMCs is known to be a decisive event in the initiation of pulmonary vascular remodeling in PAH, CHK1 could be a potential therapeutic target for PAH and IPF (Bourgeois et al., 2019; Satoh et al., 2018; Wu et al., 2022).

Activation of the ATR-CHK1 signaling in PAH-PASMCs and significant therapeutic effects observed by the inhibition of this axis in animal models mimicking PAH indicate that CHK1 may represent a new therapeutic avenue for patients with PAH. Inhibiting CHK1 signaling would block or reverse pulmonary vascular remodeling, a key pathological feature of PAH for which current approved therapies have limited efficacy. Moreover, therapeutic effects observed on cancer by inhibition of CHK1 also highlight a continuing need for developing new CHK-1 inhibitors with pharmacokinetic and pharmacodynamic properties making them suitable for use as pharmaceutical agents. Thus, the objective of the present invention is to provide such pharmaceutical agents and treatments.

### SUMMARY OF THE INVENTION

In one aspect, the present invention provides a nitrogen-containing five-membered heterocyclic compound of formula (I):

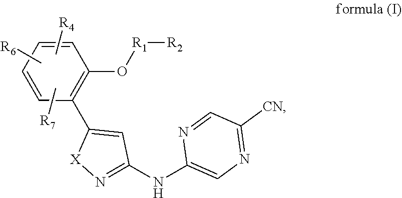

or a pharmaceutically acceptable salt thereof, wherein R₁, R₂, R₄, R₆, R₇ and κare as detailed herein.

In one aspect, the compound of formula (I) or a pharmaceutically acceptable salt thereof, is a compound of Table 1 or a pharmaceutically acceptable salt thereof, as detailed herein.

In some another aspect, the present invention provides method of treating a disease or disorder associated with this CHK kinase enzymes, more specifically CHK-1 kinase enzymes in an individual in need thereof, wherein the method comprises administering to the individual an effective amount of a compound of the present invention or a pharmaceutically acceptable salt thereof.

In some another aspect, the present invention provides method of treating cancer in an individual in need thereof, wherein the method comprises administering to the individual an effective amount of a compound of the present invention or a pharmaceutically acceptable salt thereof.

In some another aspect, the present invention provides method of treating Idiopathic Pulmonary Fibrosis (IPF) in an individual in need thereof, wherein the method comprises administering to the individual an effective amount of a compound of the present invention, or a pharmaceutically acceptable salt thereof.

In some another aspect, the present invention provides method of treating I Pulmonary Arterial Hypertension (PAH) in an individual in need thereof, wherein the method comprises administering to the individual an effective amount of a compound of the present invention, or a pharmaceutically acceptable salt thereof.

In some another aspect, the present invention provides method of treating a disease or disorder associated with this CHK kinase enzymes, more specifically CHK-1 kinase enzymes in an individual in need thereof, wherein the method comprises administering to the individual an effective amount of a compound of the present invention, or a pharmaceutically acceptable salt thereof in combination with other therapeutic agents.

In some another aspect, the present invention provides pharmaceutical compositions, comprising a compound of the present invention, or a pharmaceutically acceptable salt thereof.

In some another aspect, the present invention provides method of treating a disease or disorder associated with this CHK kinase enzymes, or more specifically CHK-1 kinase enzymes in an individual in need thereof, wherein the method comprises administering to the individual an effective amount of a pharmaceutical composition comprising a compound of the present invention, or a pharmaceutically acceptable salt thereof.

In some another aspect, the present invention provides processes for preparing compounds and intermediates thereof disclosed in the present invention. In some another aspect, the present invention provides a kit comprising the compound or a pharmaceutically acceptable salt thereof.

### DETAIL DESCRIPTION OF THE INVENTION

### Definitions

“Alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, n-pentyl, isopentyl, neopentyl, n-hexyl, 2-methylpentyl, 3-methylpentyl, 2,3-dimethylbutyl, or 2,2-dimethylbutyl, homologs and isomers of.

“Alkylene” as used herein refers to the same residues as alkyl, but having bivalency. Particular alkylene groups are those having 1 to 6 carbon atoms (a “C₁-C₆ alkylene”), 1 to 5 carbon atoms (a “C₁-C₅ alkylene”), 1 to 4 carbon atoms (a “C₁-C₄ alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Heterocycle” or “Heterocyclyl” refers to a saturated or an unsaturated non-aromatic group having from 3 to 8 annular carbon atoms and from 1 to 2 annular heteroatoms, such as nitrogen or oxygen, and the nitrogen atom(s) are optionally substituted. A heterocyclyl group may be monocyclic, bicyclic or spirocyclic 4- to 10-membered heterocyclyl. A heterocycle comprising more than one ring may be fused, spiro or bridged, or any combination thereof. In fused ring systems, one or more of the fused rings can be aryl or heteroaryl. Examples of heterocyclyl groups include, but are not limited to, aziridinyl, azetidinyl, oxetanyl, morpholinyl, azepanyl, tetrahydropyranyl, dihydropyranyl, piperidinyl, piperazinyl, pyrrolidinyl, tetrahydrofuranyl, azaspiro[3.3]heptyl, and the like.

“Ring” is partially or fully unsaturated and substituted or unsubstituted. So-called rings include single rings, interlocking rings, spiral rings, parallel rings or bridge rings. The number of atoms on the ring is usually defined as the number of elements of the ring. For example, “8-membered ring” means 8 atoms arranged in a circle. Unless otherwise specified, the ring optionally contains from 1 to 3 heteroatoms.

“CHK” refers to Checkpoint kinase, which includes CHK-1 and CHK-2. CHK refers herein specifically to CHK-1.

“Optionally substituted” unless otherwise specified means that a group may be unsubstituted or substituted by one or more (e.g., 1, 2 or 3) of the substituents listed for that group in which the substituents may be the same of different. In one embodiment, an optionally substituted group has one substituent. In another embodiment, an optionally substituted group has two substituents. In another embodiment, an optionally substituted group has three substituents.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

As used herein, “treatment” or “treating” is an approach for obtaining beneficial or desired results including clinical results. For example, beneficial or desired results include, but are not limited to, one or more of the following: decreasing symptoms resulting from the disease, increasing the quality of life of those suffering from the disease, decreasing the dose of other medications required to treat the disease, delaying the progression of the disease, and/or prolonging survival of individuals. In reference to cancers or other unwanted cell proliferation (idiopathic pulmonary fibrosis (IPF)), beneficial or desired results include shrinking a tumor (reducing tumor size); decreasing the growth rate of the tumor (such as to suppress tumor growth); reducing the number of cancer cells; inhibiting, retarding or slowing to some extent and preferably stopping cancer cell infiltration into peripheral organs; inhibiting (slowing to some extent and preferably stopping) tumor metastasis; inhibiting tumor growth; preventing or delaying occurrence and/or recurrence of tumor; and/or relieving to some extent one or more of the symptoms associated with the cancer. In some embodiments, beneficial or desired results include preventing or delaying occurrence and/or recurrence, such as of unwanted cell proliferation (idiopathic pulmonary fibrosis (IPF)).

As used herein, “delaying development of a disease” means to defer, hinder, slow, retard, stabilize, and/or postpone development of the disease (such as cancer, pulmonary arterial hypertension (PAH) and idiopathic pulmonary fibrosis (IPF)). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

As used herein, an “effective dosage” or “effective amount” of compound or salt thereof or pharmaceutical composition is an amount sufficient to effect beneficial or desired results. For prophylactic use, beneficial or desired results include results such as eliminating or reducing the risk, lessening the severity of, or delaying the onset of the disease, including biochemical, histological and/or behavioral symptoms of the disease, its complications and intermediate pathological phenotypes presenting during development of the disease. For therapeutic use, beneficial or desired results include ameliorating, palliating, lessening, delaying or decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In reference to cancers or other unwanted cell proliferation, an effective amount comprises an amount sufficient to cause a tumor to shrink and/or to decrease the growth rate of the tumor (such as to suppress tumor growth) or to prevent or delay other unwanted cell proliferation (idiopathic pulmonary fibrosis (IPF)). In reference to pulmonary arterial hypertension (PAH), an effective amount comprises an amount sufficient to prevent or delay the development of pulmonary arterial hypertension (PAH)). In some embodiments, an effective amount is an amount sufficient to delay development. In some embodiments, an effective amount is an amount sufficient to prevent or delay occurrence and/or recurrence. An effective amount can be administered in one or more administrations, in the case of cancer, the effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer.

As used herein, the term “individual” is a mammal, including humans. An individual includes, but is not limited to, human, bovine, horse, feline, canine, rodent, or primate. In some embodiments, the individual is human. The individual (such as a human) may have advanced disease or lesser extent of disease, such as low tumor burden. In some embodiments, the individual is at an early stage of a proliferative disease (such as cancer or idiopathic pulmonary fibrosis (IPF)). In some embodiments, the individual is at an advanced stage of a proliferative disease (such as an advanced cancer).

Reference to “about” a value or parameter herein includes (and describes) embodiments that are directed to that value or parameter per se. For example, description referring to “about X” includes description of “X”.

It is understood that aspects and variations described herein also include “consisting” and/or “consisting essentially of” aspects and variations.

### Compounds

The present invention provides a nitrogen-containing five-membered heterocyclic compound of Formula (I) or a pharmaceutically acceptable salt thereof:

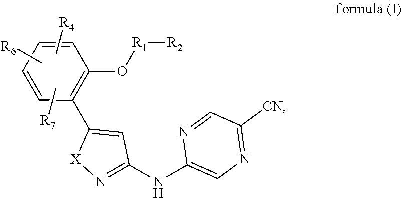

wherein,
X is selected from the group consisting of O or NH;
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄, R₆ and R₇ are independently -A-R₅ or absent;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from ═O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R₃ is selected from the group consisting of H, or C₁-C₃ alkyl; n is 0, 1 or 2;
or R₄ and R₆, together with the carbons to which they are attached, form a 5-, 6-, 7-, or, 8-membered ring, wherein said ring is partially or fully unsaturated and substituted or unsubstituted.

In some embodiments, nitrogen-containing five-membered heterocyclic derivatives of Formula (I) is

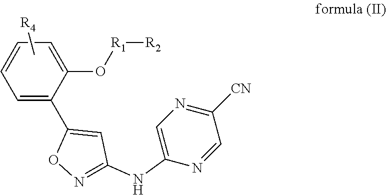

wherein,
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄ is -A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from ═O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) saturated or unsaturated monocyclic 5-6 membered heterocyclyl, wherein the heterocyclyl contains 1-3 heteroatoms independently selected from N(R₃)ₙ, O or S; optionally each R³ is selected from the group consisting of H, or C₁-C₃ alkyl; n is 0, 1 or 2;
or a pharmaceutically acceptable salt thereof.

In some embodiments, nitrogen-containing five-membered heterocyclic derivatives of Formula (II) are selected from the group consisting of:

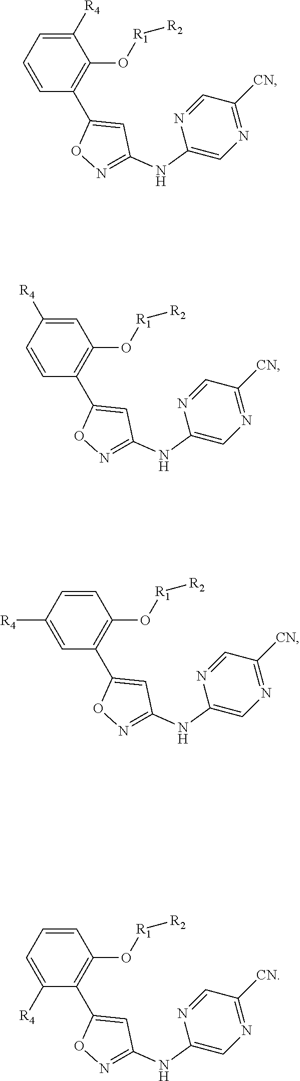

In some embodiments, a compound of Formula (II) or a pharmaceutically acceptable salt thereof, wherein,
R₄ is -A-R₅;
A is selected from:
(i) a bond; or
(ii) a saturated chain of 2 to 4 chain members in length containing at least one carbon atom chain member, at least one heteroatom chain member selected from nitrogen, and optionally one or more further carbon atom chain members and/or heteroatom chain members selected from nitrogen, oxygen, sulphur, sulphinyl and sulphonyl; the saturated chain being optionally substituted with one or more substituents selected from ═O, C₁₋₄ hydrocarbyl and fluorine wherein two hydrocarbyl substituents on the same carbon atom may optionally link to form a ring of three to five ring members;
R₅ is selected from the group consisting of
(i) H, or
(ii) saturated or unsaturated monocyclic 6 membered heterocyclyl, wherein the heterocyclyl contains 1-2 heteroatoms independently selected from N(R₃)ₙ, optionally each R³ is selected from the group consisting of H, or C₁-C₃ alkyl, n is 0, 1 or 2.

In some embodiments, a compound of Formula (II) or a pharmaceutically acceptable salt thereof, wherein,
R₄ is -A-R₅;
A is selected from:
(i) a bond; or
(ii) —(CH₂)₁₋₄, —NH—(CH₂)₁₋₃;
R₅ is selected from the group consisting of
(i) H, or

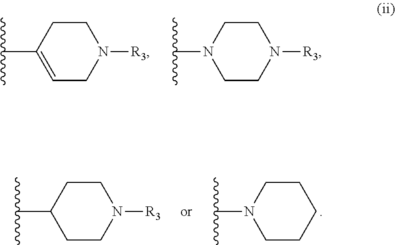

In some embodiments, nitrogen-containing five-membered heterocyclic derivatives of Formula (I) is

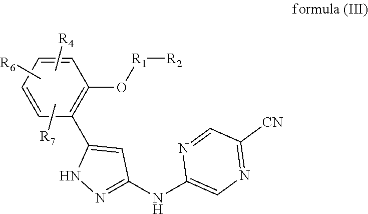

wherein,
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄, R₆ and R₇ are independently -A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R³ is selected from the group consisting of H, or C₁-C₃ alkyl; n is 0, 1 or 2;
or R₄ and R₆, together with the carbons to which they are attached, form a 5-, 6-, 7-, or, 8-membered ring, wherein said ring is partially or fully unsaturated and substituted or unsubstituted.

In some embodiments, nitrogen-containing five-membered heterocyclic derivatives of Formula (III) is

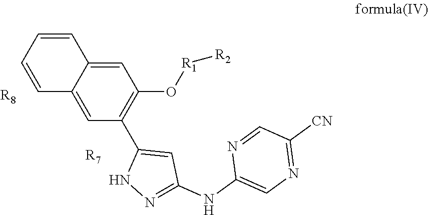

wherein R₈ is -A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R³ is selected from the group consisting of H, or C₁-C₃ alkyl; n is 0, 1 or 2.

In some embodiments, nitrogen-containing five-membered heterocyclic derivatives of Formula (IV) is

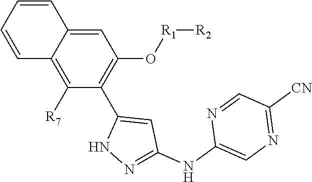

In some embodiments, a compound of Formula (I) or a pharmaceutically acceptable salt thereof, wherein,
R₁ is selected from the group consisting of C₁-C₃ alkyl, C₁-C₄ alkylene, —C₃-C₆ cycloalkyl or absent;
R₂ is selected from the group consisting of H, —NH₂, unsaturated or saturated monocyclic, or spirocyclic 4- to 6-membered heterocyclyl, wherein the heterocyclyl contains 1-2 heteroatoms independently selected from N(R₃)ₙ, and O; In some embodiments, a compound of Formula (I) or a pharmaceutically acceptable salt thereof, wherein, R₁ is selected from the group consisting methyl, ethyl, n-propyl, isopropyl, —CH₂—, —CH₂CH₂—, —CH₂CH₂CH₂—, —CH₂CH₂CH₂CH₂—, cyclopropyl, cyclobutyl, cyclopentyl, cyclohexyl, or absent.

In some embodiments, a compound of Formula (I) or a pharmaceutically acceptable salt thereof, wherein, R₂ is selected from the group consisting of H, —NH₂,

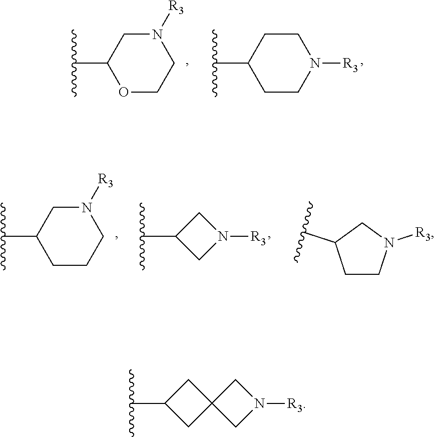

In some embodiments, a compound of Formula (I) or a pharmaceutically acceptable salt thereof, wherein, R₃ is selected from the group consisting of H, methyl, ethyl, n-propyl, isopropyl, n-butyl, t-butyl, isobutyl, sec-butyl, n-pentyl, isopentyl, neopentyl, n-hexyl, 2-methylpentyl, 3-methylpentyl, 2,3-dimethylbutyl, or 2,2-dimethylbutyl.

In some embodiments, a compound of Formula (I) or a pharmaceutically acceptable salt thereof, wherein the compound is selected from Table 1.

TABLE 1
Compound

| Compound No. | Structure | Chemical Name |
|---|---|---|
| Compound 1.1 | 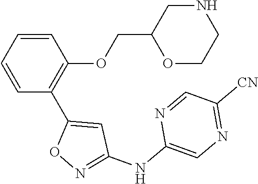 [SMILES: N#Cc1cnc(Nc2cc(-c3ccccc3OCC3CNCCO3)on2)cn1] | 5-(5-(2-(morpholin-2- ylmethoxy)phenyl)- isoxazol-3-ylamino)- pyrazine-2-carbonitrile |
| Compound 1.2 | 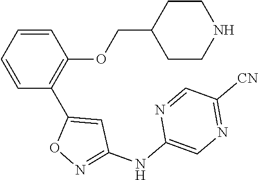 [SMILES: N#Cc1cnc(Nc2cc(-c3ccccc3OCC3CCNCC3)on2)cn1] | 5-(5-(2-(piperidin-4-yl- methoxy)phenyl)isox- azol-3-ylamino)pyr- azine-2-carbonitrile |
| Compound 1.3 | 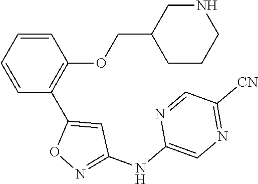 [SMILES: N#Cc1cnc(Nc2cc(-c3ccccc3OCC3CCCNC3)on2)cn1] | 5-(5-(2-(piperidin-3-yl- methoxy)phenyl)isox- azol-3-ylamino)pyr- azine-2-carbonitrile |
| Compound 1.4 | 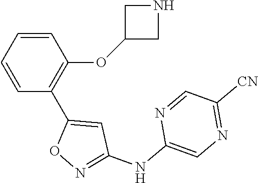 [SMILES: N#Cc1cnc(Nc2cc(-c3ccccc3OC3CNC3)on2)cn1] | 5-(5-(2-(azetidin-3-yl- oxy)phenyl)isoxazol- 3-ylamino)pyrazine-2- carbonitrile |
| Compound 1.5 | 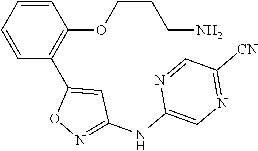 [SMILES: N#Cc1cnc(Nc2cc(-c3ccccc3OCCCN)on2)cn1] | 5-(5-(2-(3-aminoprop- oxy)phenyl)isoxazol-3- ylamino)pyrazine-2- carbonitrile |
| Compound 1.6 | 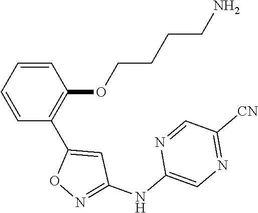 | 5-(5-(2-(4-aminobut- oxy)phenyl)isoxazol-3- ylamino)pyrazine-2- carbonitrile |
| Compound 1.7 | 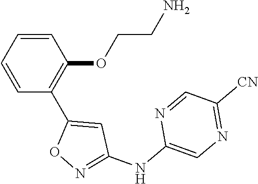 | 5-(5-(2-(2-amino- ethoxy)phenyl)isox- azol-3-ylamino)pyr- azine-2-carbonitrile |
| Compound 1.8 | 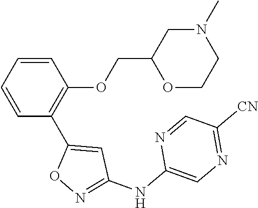 [SMILES: CN1CCOC(COc2ccccc2-c2cc(Nc3cnc(C#N)cn3)no2)C1] | 5-(5-(2-((4-methyl- morpholin-2-yl)meth- oxy)phenyl)isoxazol- 3-ylamino)-pyrazine- 2-carbonitrile |
| Compound 1.9 | 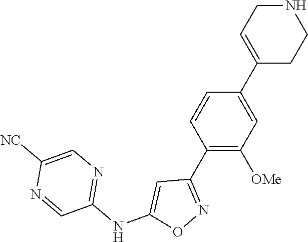 [SMILES: COc1cc(C2=CCNCC2)ccc1-c1cc(Nc2cnc(C#N)cn2)on1] | 5-(3-(2-methoxy-4- (1,2,3,6-tetrahydropyr- idin-4-yl)phenyl)- isoxazol-5-ylamino)- pyrazine-2-carbonitrile |
| Compound 1.10 | 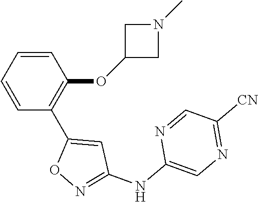 | 5-(5-(2-(1-methylazeti- din-3-yloxy)phenyl)- isoxazol-3-ylamino)- pyrazine-2-carbonitrile |
| Compound 1.11 | 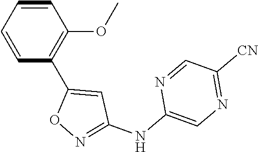 | 5-(5-(2-methoxyphen- yl)isoxazol-3-ylamino)- pyrazine-2-carbonitrile |
| Compound 1.12 | 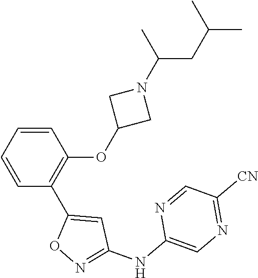 [SMILES: CC(C)CC(C)N1CC(Oc2ccccc2-c2cc(Nc3cnc(C#N)cn3)no2)C1] | 5-(5-(2-(1-(4-methyl- pentan-2-yl)azetidin-3- yloxy)phenyl)isoxazol- 3-ylamino)pyrazine-2- carbonitrile |
| Compound 1.13 | 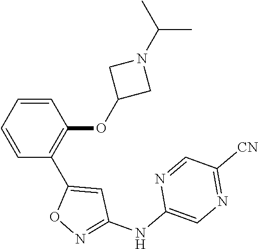 | 5-(5-(2-(1-isopropyl- azetidin-3-yloxy)- phenyl)isoxazol-3-yl- amino)pyrazine-2- carbonitrile |
| Compound 1.14 | 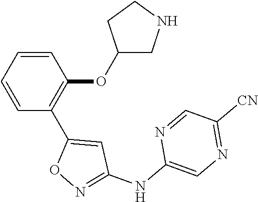 | 5-(5-(2-(pyrrolidin-3- yloxy)phenyl)isoxazol- 3-ylamino)pyrazine-2- carbonitrile |
| Compound 1.15 | 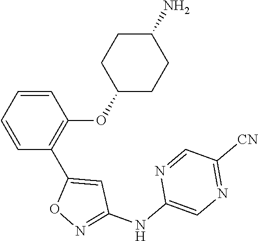 [SMILES: N#Cc1cnc(Nc2cc(-c3ccccc3O[C@H]3CC[C@@H](N)CC3)on2)cn1] | 5-(5-(2-((1s,4s)-4- aminocyclohexyloxy)- phenyl)isoxazol-3-yl- amino)pyrazine-2- carbonitrile |
| Compound 1.16 | 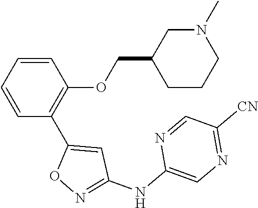 | 5-(5-(2-((1-methyl- piperidin-3-yl)meth- oxy)phenyl)isoxazol- 3-ylamino)pyrazine- 2-carbonitrile |
| Compound 1.17 | 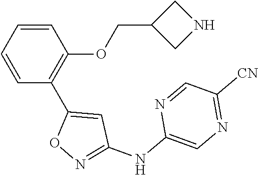 [SMILES: N#Cc1cnc(Nc2cc(-c3ccccc3OCC3CNC3)on2)cn1] | 5-(5-(2-(azetidin-3-yl- methoxy)phenyl)isox- azol-3-ylamino)pyr- azine-2-carbonitrile |
| Compound 1.18 | 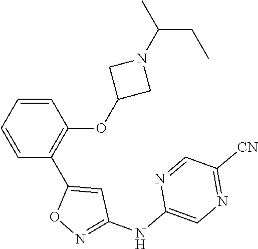 [SMILES: CCC(C)N1CC(Oc2ccccc2-c2cc(Nc3cnc(C#N)cn3)no2)C1] | 5-(5-(2-(1-sec-butyl- azetidin-3-yloxy)phen- yl)isoxazol-3-ylamino)- pyrazine-2-carbonitrile |
| Compound 1.19 | 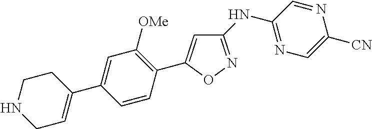 [SMILES: COc1cc(C2=CCNCC2)ccc1-c1cc(Nc2cnc(C#N)cn2)no1] | 5-(5-(2-methoxy-4- (1,2,3,6-tetrahydropyr- idin-4-yl)phenyl)isox- azol-3-ylamino)pyr- azine-2-carbonitrile |
| Compound 1.20 | 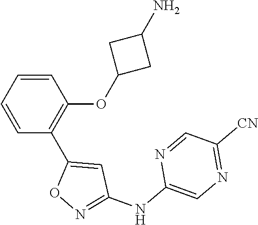 [SMILES: N#Cc1cnc(Nc2cc(-c3ccccc3OC3CC(N)C3)on2)cn1] | 5-(5-(2-(3-aminocyclo- butoxy)phenyl)isox- azol-3-ylamino)pyr- azine-2-carbonitrile |
| Compound 1.21 | 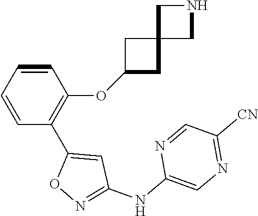 | 5-(5-(2-(2-azaspiro[3.3] heptan-6-yloxy)phen- yl)isoxazol-3-ylamino)- pyrazine-2-carbonitrile |
| Compound 1.22 | 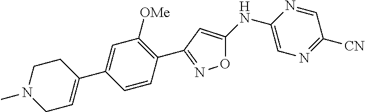 [SMILES: COc1cc(C2=CCN(C)CC2)ccc1-c1cc(Nc2cnc(C#N)cn2)on1] | 5-(3-(2-methoxy-4-(1- methyl-1,2,3,6-tetra- hydropyridin-4-yl)- phenyl)isoxazol-5-yl- amino)pyrazine-2- carbonitrile |
| Compound 1.23 | 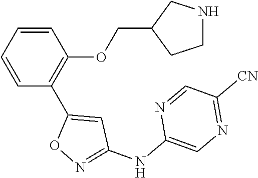 [SMILES: N#Cc1cnc(Nc2cc(-c3ccccc3OCC3CCNC3)on2)cn1] | 5-(5-(2-(pyrrolidin-3- ylmethoxy)phenyl)isox- azol-3-ylamino)pyr- azine-2-carbonitrile |
| Compound 2.1 | 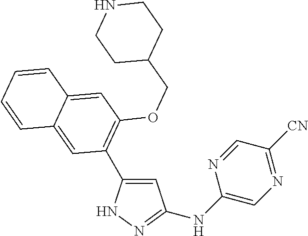 [SMILES: N#Cc1cnc(Nc2cc(-c3cc4ccccc4cc3OCC3CCNCC3)[nH]n2)cn1] | 5-(5-(3-(piperidin-4-yl- methoxy)naphthalen-2- yl)-1H-pyrazol-3-yl- amino)pyrazine-2- carbonitrile |
| Compound 2.2 | 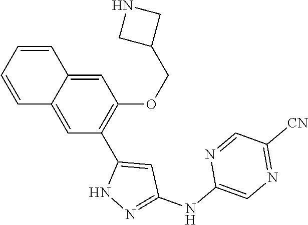 [SMILES: N#Cc1cnc(Nc2cc(-c3cc4ccccc4cc3OCC3CNC3)[nH]n2)cn1] | 5-(5-(3-(azetidine-3-yl- methoxy)naphthalen-2- yl)-1H-pyrazol-3-yl- amino)pyrazine-2- carbonitrile |
| Compound 2.3 | 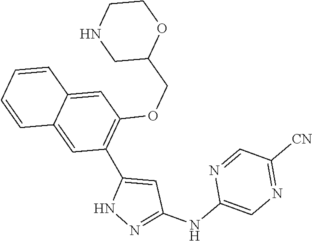 [SMILES: N#Cc1cnc(Nc2cc(-c3cc4ccccc4cc3OCC3CNCCO3)[nH]n2)cn1] | 5-(5-(3-(morpholin-2- ylmethoxy)naphthalen- 2-yl)-1H-pyrazol-3-yl- amino)pyrazine-2- carbonitrile |
| Compound 2.4 | 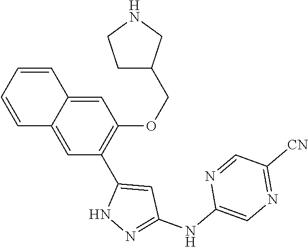 [SMILES: N#Cc1cnc(Nc2cc(-c3cc4ccccc4cc3OCC3CCNC3)[nH]n2)cn1] | 5-(5-(3-(pyrrolidin-3- ylmethoxy)naphthalen- 2-yl)-1H-pyrazol-3-yl- amino)pyrazine-2- carbonitrile |
| Compound 2.5 | 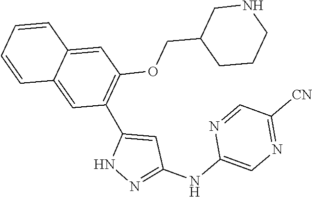 [SMILES: N#Cc1cnc(Nc2cc(-c3cc4ccccc4cc3OCC3CCCNC3)[nH]n2)cn1] | 5-(5-(3-(piperidin-3-yl- methoxy)naphthalen-2- yl)-1H-pyrazol-3-yl- amino)pyrazine-2- carbonitrile |
| Compound 2.6 | 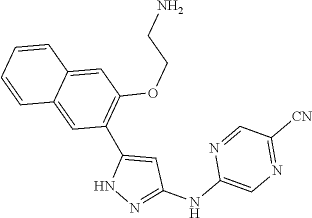 [SMILES: N#Cc1cnc(Nc2cc(-c3cc4ccccc4cc3OCCN)[nH]n2)cn1] | 5-((5-(3-(2-amino- ethoxy)naphthalen-2- yl)-1H-pyrazol-3-yl)- amino)pyrazine-2- carbonitrile |
| Compound 2.7 | 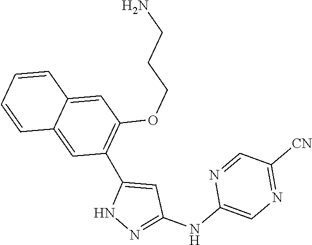 [SMILES: N#Cc1cnc(Nc2cc(-c3cc4ccccc4cc3OCCCN)[nH]n2)cn1] | 5-(5-(3-(3-aminoprop- oxy)naphthalen-2-yl)- 1H-pyrazol-3-ylamino)- pyrazine-2-carbonitrile |

Also provided are salts of compounds referred to herein, such as pharmaceutically acceptable salts. The invention also includes any or all of the stereochemical forms, including any enantiomeric or diastereomeric forms, and any tautomers or other forms of the compounds described.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form. Unless otherwise stated, “substantially pure” intends a composition that contains no more than 75% impurity, wherein the impurity denotes a compound other than the compound comprising the majority of the composition or a salt thereof. In some embodiments, a composition of substantially pure compound or a salt thereof is provided wherein the composition contains no more than 25%, 20%, 15%, 10%, or 5% impurity. In some embodiments, a composition of substantially pure compound or a salt thereof is provided wherein the composition contains or no more than 3%, 2%, 1% or 0.5% impurity.

Representative compounds of formula (I) are listed in table 1. It is understood that individual enantiomers and diastereomers are included in the generic compound structures shown in table 1. Specific synthetic methods for preparing compounds No. 1.1 to 1.22 and 2.1 to 2.7 of table 1 are provided example herein.

In some embodiments, provided herein are compounds described in table 1, or a salt, polymorph, solvate, enantiomer, stereoisomer or tautomer thereof, and uses thereof.

The embodiments and variations described herein are suitable for compounds of any formulae detailed herein, where applicable.

Representative examples of compounds detailed herein, including intermediates and final compounds according to the present disclosure are depicted herein. It is understood that in one aspect, any of the compounds may be used in the methods detailed herein, including, where applicable, intermediate compounds that may be isolated and administered to an individual.

The compounds depicted herein may be present as salts even if salts are not depicted and it is understood that the present disclosure embraces all salts and solvates of the compounds depicted here, as well as the non-salt and non-solvate form of the compound, as is well understood by the skilled in the art. In some embodiments, the salts of the compounds provided herein are pharmaceutically acceptable salts. In some embodiment, the pharmaceutically acceptable salt is formate salt.

Where tautomeric forms may be present for any of the compounds described herein, each and every tautomeric form is intended even though only one or some of the tautomeric forms may be explicitly depicted. The tautomeric forms specifically depicted may or may not be the predominant forms in solution or when used according to the methods described herein.

The present disclosure also includes any or all of the stereochemical forms, including any enantiomeric or diastereomeric forms of the compounds described. The structure or name is intended to embrace all possible stereoisomers of a compound depicted. All forms of the compounds are also embraced by the invention, such as crystalline or non-crystalline forms of the compounds. Compositions comprising a compound of the invention are also intended, such as a composition of substantially pure compound, including a specific stereochemical form thereof, or a composition comprising mixtures of compounds of the invention in any ratio, including two or more stereochemical forms, such as in a racemic or non-racemic mixture.

The invention also intends isotopically-labeled and/or isotopically-enriched forms of compounds described herein. The compounds herein may contain unnatural proportions of atomic isotopes at one or more of the atoms that constitute such compounds. In some embodiments, the compound is isotopically-labeled, such as an isotopically-labeled compound of the formula (I) or variations thereof described herein, where a fraction of one or more atoms are replaced by an isotope of the same element. Exemplary isotopes that can be incorporated into compounds of the invention include isotopes of hydrogen, carbon, nitrogen, oxygen, phosphorus, sulfur, chlorine, such as ²H, ³H, ¹¹C, ¹³C, ¹⁴C, ¹³N, ¹⁵O, ¹⁷O, ³²P, ³⁵S, ¹⁸F, ³⁶Cl. Certain isotope labeled compounds (e.g. ³H and ¹⁴C) are useful in compound or substrate tissue distribution studies. Incorporation of heavier isotopes such as deuterium (²H) can afford certain therapeutic advantages resulting from greater metabolic stability, for example, increased in vivo half-life, or reduced dosage requirements and, hence may be preferred in some instances.

Isotopically-labeled compounds of the present invention can generally be prepared by standard methods and techniques known to those skilled in the art or by procedures similar to those described in the accompanying Examples substituting appropriate isotopically-labeled reagents in place of the corresponding non-labeled reagent.

The invention also includes any or all metabolites of any of the compounds described. The metabolites may include any chemical species generated by a biotransformation of any of the compounds described, such as intermediates and products of metabolism of the compound, such as would be generated in vivo following administration to a human.

Articles of manufacture comprising a compound described herein, or a salt or solvate thereof, in a suitable container are provided. The container may be a vial, jar, ampoule, preloaded syringe, i.v. bag, and the like.

Preferably, the compounds detailed herein are orally bioavailable. However, the compounds may also be formulated for parenteral (e.g., intravenous) administration.

One or several compounds described herein can be used in the preparation of a medicament by combining the compound or compounds as an active ingredient with a pharmacologically acceptable carrier, which are known in the art. Depending on the therapeutic form of the medication, the carrier may be in various forms. In one variation, the manufacture of a medicament is for use in any of the methods disclosed herein, e.g., for the treatment of cancer.

### General Synthetic Schemes

The compounds of the invention may be prepared by a number of processes as generally described below and more specifically in the Examples hereinafter (such as the schemes provided in the Examples below). In the following process descriptions, the symbols when used in the formulae depicted are to be understood to represent those groups described above in relation to the formulae herein.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g., a racemate, and an appropriate chiral compound. The diastereomers may then be separated by any convenient means, for example by crystallization and the desired enantiomer recovered. In another resolution process, a racemate may be separated using chiral High Performance Liquid Chromatography. Alternatively, if desired a particular enantiomer may be obtained by using an appropriate chiral intermediate in one of the processes described.

Chromatography, recrystallization and other conventional separation procedures may also be used with intermediates or final products where it is desired to obtain a particular isomer of a compound or to otherwise purify a product of a reaction.

### Pharmaceutical Compositions and Formulations

Pharmaceutical compositions of any of the compounds detailed herein are embraced by this disclosure. Thus, the present disclosure includes pharmaceutical compositions comprising a compound as detailed herein or a salt thereof and a pharmaceutically acceptable carrier or excipient. In one aspect, the pharmaceutically acceptable salt is an acid addition salt, such as a salt formed with an inorganic or organic acid. In some embodiment, the pharmaceutically acceptable salt is formate salt.

Pharmaceutical compositions may take a form suitable for oral, buccal, parenteral, nasal, topical or rectal administration or a form suitable for administration by inhalation.

A compound as detailed herein may in one aspect be in a purified form and compositions comprising a compound in purified forms are detailed herein. Compositions comprising a compound as detailed herein or a salt thereof are provided, such as compositions of substantially pure compounds. In some embodiments, a composition containing a compound as detailed herein or a salt thereof is in substantially pure form.

In one variation, the compounds herein are synthetic compounds prepared for administration to an individual. In another variation, compositions are provided containing a compound in substantially pure form. In another variation, the present disclosure embraces pharmaceutical compositions comprising a compound detailed herein and a pharmaceutically acceptable carrier. In another variation, methods of administering a compound are provided. The purified forms, pharmaceutical compositions and methods of administering the compounds are suitable for any compound or form thereof detailed herein.

A compound detailed herein or salt thereof may be formulated for any available delivery route, including an oral, mucosal (e.g., nasal, sublingual, vaginal, buccal or rectal), parenteral (e.g., intramuscular, subcutaneous or intravenous), topical or transdermal delivery form. A compound or salt thereof may be formulated with suitable carriers to provide delivery forms that include, but are not limited to, tablets, caplets, capsules (such as hard gelatin capsules or soft elastic gelatin capsules), cachets, troches, lozenges, gums, dispersions, suppositories, ointments, cataplasms (poultices), pastes, powders, dressings, creams, solutions, patches, aerosols (e.g., nasal spray or inhalers), gels, suspensions (e.g., aqueous or non-aqueous liquid suspensions, oil-in-water emulsions or water-in-oil liquid emulsions), solutions and elixirs.

Compositions comprising a compound provided herein are also described. In one variation, the composition comprises a compound or salt thereof and a pharmaceutically acceptable carrier or excipient. In another variation, a composition of substantially pure compound is provided.

### Methods of Use

Compounds and compositions detailed herein, such as a pharmaceutical composition containing a compound of any formula provided herein or a salt thereof and a pharmaceutically acceptable carrier or excipient, may be used in methods of administration and treatment as provided herein. The compounds and compositions may also be used in in vitro methods, such as in vitro methods of administering a compound or composition to cells for screening purposes and/or for conducting quality control assays.

Provided herein is a method of treating a disease in an individual comprising administering an effective amount of a compound of the present invention (collectively, a compound of formula (I) or any embodiment, variation or aspect thereof or the present compounds or the compounds detailed or described herein) or a pharmaceutically acceptable salt thereof, to the individual. Further provided herein is a method of treating a proliferative disease in an individual, comprising administering an effective amount of the compound of the present invention or a pharmaceutically acceptable salt thereof, to the individual. Also provided herein is a method of treating cancer, pulmonary arterial hypertension (PAH) or idiopathic pulmonary fibrosis (IPF) in an individual comprising administering an effective amount of the compound of formula (I) or a pharmaceutically acceptable salt thereof, to the individual. In some embodiments, the compound is administered to the individual according to a dosage and/or method of administration described herein.

Another aspect of the invention relates to a method of treating a disease or disorder associated with Checkpoint kinase. The method involves administering to a patient in need of a treatment for diseases or disorders associated with

Checkpoint kinase an effective amount of the compositions and compounds of the present invention.

Another aspect of the invention is directed to a method inhibiting Checkpoint kinase. The method involves administering to a patient in need thereof an effective amount of the compositions or compounds of the present invention (collectively, a compound of formula (I).

The use of a compound of the present invention for the manufacture of a medicament for the treatment of a proliferative disease such as cancer, pulmonary arterial hypertension (PAH) and idiopathic pulmonary fibrosis (IPF).

The use of a compound of the present invention for the manufacture of a medicament for the treatment of cancer, wherein the cancer is selected from carcinomas, for example carcinomas of the bladder, breast, colon, kidney, epidermis, liver, lung, oesophagus, gall bladder, ovary, pancreas, stomach, cervix, thyroid, prostate, gastrointestinal system, or skin, hematopoieitic tumours such as leukaemia, B-cell lymphoma, T-cell lymphoma, Hodgkin's lymphoma, non-Hodgkin's lymphoma, hairy cell lymphoma, or Burkett's lymphoma; hematopoieitic tumours of myeloid lineage, for example acute and chronic myelogenous leukaemias, myelodysplastic syndrome, or promyelocytic leukaemia; p53 negative or mutated tumours; MYC oncogene-driven cancer such as B-cell lymphoma, leukemia, neuroblastoma, breast cancer or lung cancer; thyroid follicular cancer; tumours of mesenchymal origin, for example fibrosarcoma or habdomyosarcoma; tumours of the central or peripheral nervous system, for example astrocytoma, neuroblastoma, glioma or schwannoma; melanoma; seminoma; teratocarcinoma; osteosarcoma; xeroderma pigmentosum; keratoctanthoma; thyroid follicular cancer; Ewing's sarcoma or Kaposi's sarcoma. The use of a compound of the present invention (collectively, a compound of formula (I) for the manufacture of a medicament for the treatment of cancer, wherein the cancer is one which is characterized by a defective DNA repair mechanism or defective cell cycle.

The use of a compound of the present invention (collectively, a compound of formula (I) for the manufacture of a medicament for the treatment of anidiopathic pulmonary fibrosis (IPF). IPF is a chronic scarring lung disease characterized by progressive and irreversible decline in lung function. It is closely associated with concomitantly occurring pulmonary arterial hypertension, wherein there is aberrant proliferation of arterial cells, vascular remodeling, inflammation, and differentiation of fibroblasts into myofibroblasts. The goal of treatment in IPF is to manage the symptoms, retard disease progression, prevent acute exacerbations, and prolong survival.

The use of a compound of the present invention for the manufacture of a medicament for the treatment of pulmonary arterial hypertension (PAH). Pulmonary arterial hypertension (PAH) is a debilitating disease associated with progressive vascular remodeling of distal pulmonary arteries leading to elevation of pulmonary artery pressure, right ventricular hypertrophy, and death. Although presenting high levels of DNA damage that normally jeopardize their viability, pulmonary artery smooth muscle cells (PASMCs) from patients with PAH exhibit a cancer-like proproliferative and apoptosis-resistant phenotype accounting for vascular lumen obliteration. CHK1 expression is markedly increased in isolated PASMCs and distal PAs from patients with PAH.

Using a pharmacological and molecular loss of function approach, it has been depicted that CHK1 promotes PAH-PASMCs proliferation and resistance to apoptosis. In-vivo, pharmacological inhibition of CHK1 significantly reduces vascular remodeling and improves hemodynamic parameters in experimental rat models of PAH.

The disclosed compounds of the present invention can be administered in effective amounts to treat or prevent a disorder and/or prevent the development thereof in subjects.

The invention can be further understood by reference to the following examples, which are provided by way of illustration and are not meant to be limiting.

### EXAMPLES

### Example-1: Synthesis of 5-(5-(2-(morpholin-2-ylmethoxy)phenyl) isoxazol-3-ylamino) pyrazine-2-carbonitrile (Compound 1.1)

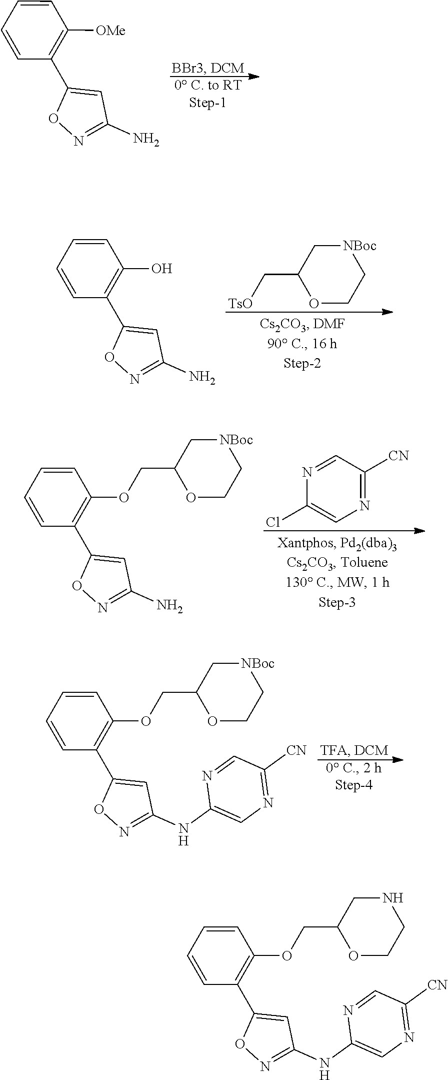

Step-1: Synthesis of 2-(3-aminoisoxazol-5-yl) phenol: To a solution of 5-(2-methoxyphenyl) isoxazol-3-amine (synthesized as described in WO2020/002587A1) (1.4 g, 7.36 mmol, 1.0 eq) in DCM (10 mL) at 0° C. was added boron tribromide (2.1 mL, 22.08 mmol, 3.0 eq). The resultant reaction mixture was allowed to reach RT and stirred for 10 h. Product formation was confirmed by TLC and LCMS. After the completion of reaction, the reaction mixture was added drop wise to ice water (30 mL). The resulting product was filtered off and dried under vacuum to obtain 2-(3-aminoisoxazol-5-yl) phenol (980 mg). LCMS: 177.3 [M+1]⁺

Step-2: Synthesis of tert-butyl 2-((2-(3-aminoisoxazol-5-yl) phenoxy)methyl) morpholine-4-carboxylate: To a solution of 2-(3-aminoisoxazol-5-yl) phenol (0.426 g, 2.42 mmol, 1.0 eq) in DMF (6 mL) was added tert-butyl 2-(tosyloxymethyl) morpholine-4-carboxylate (synthesized as shown in WO 2014151616 A1) (988 mg, 2.66 mmol, 1.1 eq) and Cs₂CO₃ (1.1 g, 3.39 mmol, 1.4 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (420 mg). LCMS: 376.3 [M+1]⁺

Step-3: Synthesis of tert-butyl 2-((2-(3-(5-cyanopyrazin-2-ylamino) isoxazol-5-yl) phenoxy)methyl) morpholine-4-carboxylate: A solution of tert-butyl 2-((2-(3-aminoisoxazol-5-yl) phenoxy)methyl) morpholine-4-carboxylate (300 mg, 0.8 mmol, 1.0 eq) and 5-chloropyrazine-2-carbonitrile (134 mg, 0.96 mmol, 1.2 eq) in toluene (9 mL) was purged with nitrogen for 5 minutes followed by addition of Cs₂CO₃ (780 mg, 2.4 mmol, 3.0 eq). The resultant reaction mixture was again purged for 5 minutes, then Xantphos (28 mg, 0.048 mmol, 0.06 eq) and Pd₂dba₃ (58 mg, 0.064 mmol, 0.08 eq) was added and the reaction mixture was subjected to microwave for 1 hour at 130° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate-hexane solvent system to obtain the desired compound (120 mg). LCMS: 479.2 [M+1]⁺

Step-4: Synthesis of 5-(5-(2-(morpholin-2-ylmethoxy)phenyl) isoxazol-3-ylamino) pyrazine-2-carbonitrile: To a solution of tert-butyl 2-((2-(3-(5-cyanopyrazin-2-ylamino) isoxazol-5-yl) phenoxy)methyl) morpholine-4-carboxylate (100 mg, 0.209 mmol, 1.0 eq) in DCM (5 mL), TFA (0.4 mL) was added at 0° C. and kept for stirring for 2 h. Then the reaction mixture was concentrated under reduced pressure to get the crude. The crude was purified by R-HPLC to yield desired compound \ (18 mg, formate salt). LCMS: 379.4 [M+1]⁺UPLC: 97.99 @ 220 nm 99.22 @ 254 nm. ¹H NMR (400 MHZ, DMSO-d6, after D₂O addition) δ 8.73 (s, 1H), 8.55 (s, 1H), 8.32 (s, 1H, formate), 7.84 (d, J=7.7 Hz, 1H), 7.48 (t, J=7.9 Hz, 1H), 7.30 (s, 1H), 7.20-7.08 (m, 2H), 4.22-4.09 (m, 2H), 3.97-3.89 (m, 2H), 3.67 (t, J=11.8 Hz, 1H), 3.17 (t, J=11.9 Hz, 1H), 3.01 (d, J=12.8 Hz, 1H), 2.85 (q, J=11.4 Hz, 2H).

### Example-2: Synthesis of 5-(5-(2-(piperidin-4-ylmethoxy)phenyl) isoxazol-3-ylamino) pyrazine-2-carbonitrile (Compound 1.2)

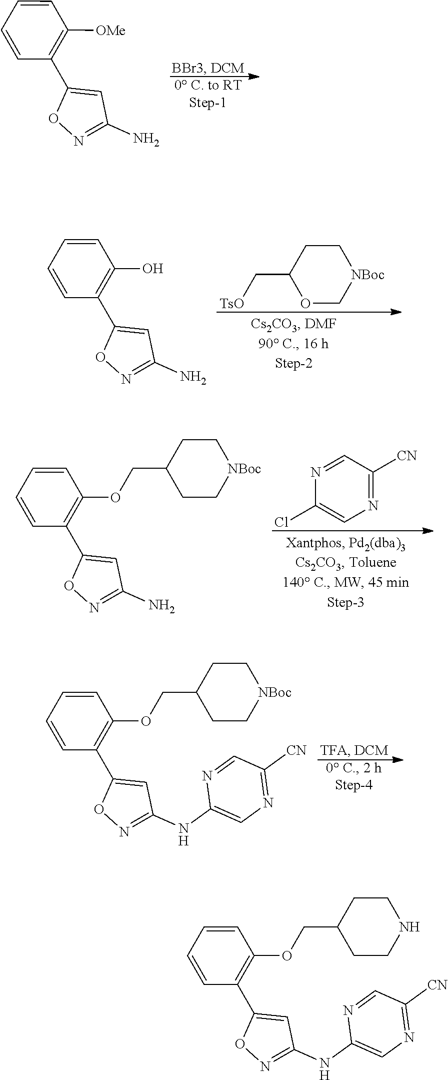

Step-1: Synthesis of 2-(3-aminoisoxazol-5-yl) phenol: Same as described in Compound 1.1

Step-2: Synthesis of tert-butyl 4-((2-(3-aminoisoxazol-5-yl) phenoxy)methyl) piperidine-1-carboxylate: To a solution of 2-(3-aminoisoxazol-5-yl) phenol (476 mg, 2.7 mmol, 1.0 eq) in DMF (8 mL) was added tert-butyl 4-(tosyloxymethyl) piperidine-1-carboxylate (synthesized as shown in WO 2020028724 A1) (998 mg, 2.7 mmol, 1.0 eq) and Cs₂CO₃ (1.14 g, 3.5 mmol, 1.3 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (420 mg). LCMS: 374.3 [M+1]⁺

Step-3: Synthesis of tert-butyl 4-((2-(3-(5-cyanopyrazin-2-ylamino) isoxazol-5-yl) phenoxy)methyl) piperidine-1-carboxylate: A solution of tert-butyl 4-((2-(3-aminoisoxazol-5-yl) phenoxy)methyl) piperidine-1-carboxylate (150 mg, 0.40 mmol, 1.0 eq) and 5-chloropyrazine-2-carbonitrile (67 mg, 0.48 mmol, 1.2 eq) in toluene (5 mL) was purged with nitrogen for 5 minutes followed by addition of Cs₂CO₃ (393 mg, 1.20 mmol, 3.0 eq). The resultant reaction mixture was again purged for 5 minutes, then Xantphos (14 mg, 0.024 mmol, 0.06 eq) and Pd₂dba₃ (29 mg, 0.032 mmol, 0.08 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate: hexane solvent system to obtain the desired compound (60 mg). LCMS: 477.3 [M+1]⁺

Step-4: Synthesis of 5-(5-(2-(piperidin-4-ylmethoxy)phenyl) isoxazol-3-ylamino) pyrazine-2-carbonitrile: To a solution of tert-butyl 4-((2-(3-(5-cyanopyrazin-2-ylamino) isoxazol-5-yl) phenoxy)methyl) piperidine-1-carboxylate (60 mg, 0.126 mmol, 1.0 eq) in DCM (5 ML), TFA (0.4 mL) was added at 0° C. and kept for stirring for 2 h. Then the reaction mixture was concentrated under reduced pressure to get the crude. The crude was purified by R-HPLC to yield desired compound (10 mg, formate salt). LCMS: 377.3 [M+1]⁺UPLC: 96.32 @ 220 nm 96.41 @ 254 nm; H NMR (400 MHZ, Methanol-d4) δ 8.65 (d, J=11.6 Hz, 2H), 8.55 (s, 1H, formate), 7.90 (dd, J=8.0, 1.8 Hz, 1H), 7.53-7.45 (m, 1H), 7.26 (s, 1H), 7.20 (d, J=8.4 Hz, 1H), 7.12 (t, J=7.6 Hz, 1H), 4.11 (d, J=6.4 Hz, 2H), 3.46 (d, J=12.0 Hz, 2H), 3.07 (td, J=12.9, 2.9 Hz, 2H), 2.32 (s, 1H), 2.19 (d, J=14.3 Hz, 2H), 1.69-1.56 (m, 2H).

### Example-3: Synthesis of 5-(5-(2-(piperidin-3-ylmethoxy)phenyl) isoxazol-3-ylamino) pyrazine-2-carbonitrile (Compound 1.3)

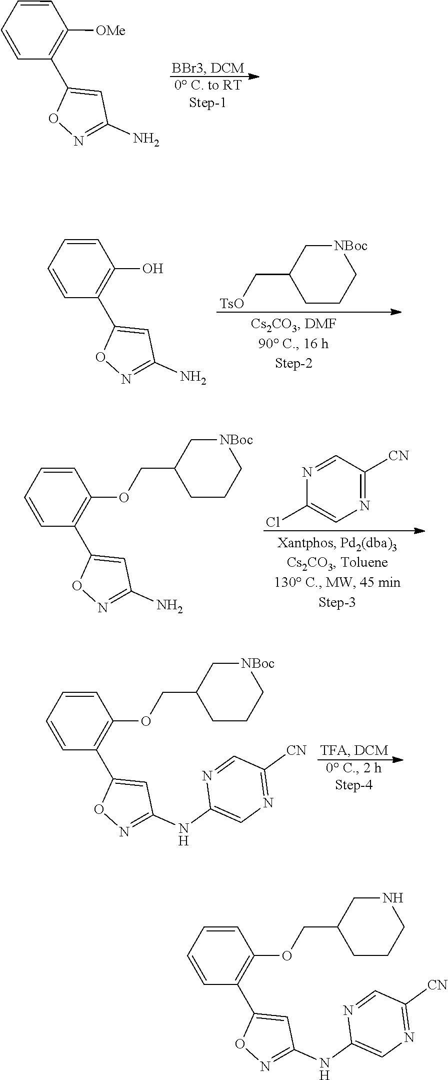

Step-1: Synthesis of 2-(3-aminoisoxazol-5-yl) phenol: Same as described in Compound 1.1

Step-2: Synthesis of tert-butyl 3-((2-(3-aminoisoxazol-5-yl) phenoxy)methyl) piperidine-1-carboxylate: To a solution of 2-(3-aminoisoxazol-5-yl) phenol (286 mg, 1.62 mmol, 1.0 eq) in DMF (8 mL) was added tert-butyl 3-(tosyloxymethyl) piperidine-1-carboxylate (synthesized as shown in J. Med. Chem. 2020, 63 (19), 11054-84) (600 mg, 1.62 mmol, 1.0 eq) and Cs₂CO₃ (688 mg, 2.11 mmol, 1.3 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (240 mg). LCMS: 374.3 [M+1]⁺

Step-3: Synthesis of tert-butyl 3-((2-(3-(5-cyanopyrazin-2-ylamino) isoxazol-5-yl) phenoxy)methyl) piperidine-1-carboxylate: A solution of tert-butyl 3-((2-(3-aminoisoxazol-5-yl) phenoxy)methyl) piperidine-1-carboxylate (150 mg, 0.40 mmol, 1.0 eq) and 5-chloropyrazine-2-carbonitrile (67 mg, 0.48 mmol, 1.2 eq) in toluene (5 mL) was purged with nitrogen for 5 minutes followed by addition of Cs₂CO₃ (393 mg, 1.20 mmol, 3.0 eq). The resultant reaction mixture was again purged for 5 minutes, then Xantphos (14 mg, 0.024 mmol, 0.06 eq) and Pd₂dba₃ (29 mg, 0.032 mmol, 0.08 eq) was added and the reaction mixture was subjected to microwave for 45 minutes at 130° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired compound. LCMS: 477.3 [M+1]⁺

Step-4: Synthesis of 5-(5-(2-(piperidin-3-ylmethoxy)phenyl) isoxazol-3-ylamino) pyrazine-2-carbonitrile: To a solution of tert-butyl 3-((2-(3-(5-cyanopyrazin-2-ylamino) isoxazol-5-yl) phenoxy)methyl) piperidine-1-carboxylate (60 mg, 0.126 mmol, 1.0 eq) in DCM (5 mL), TFA (0.4 mL) was added at 0° C. and kept for stirring for 2 h. Then the reaction mixture was concentrated under reduced pressure to get the crude. The crude was purified by R-HPLC to yield desired compound (15 mg, formate salt). LCMS: 377.3 [M+1]⁺UPLC: 99.31 @ 220 nm 99.57 @ 254 nm; ¹H NMR (400 MHZ, Methanol-d₄) δ 8.64 (d, J=8.8 Hz, 2H), 8.55 (s, 1H, formate), 7.91-7.84 (m, 1H), 7.49 (t, J=7.7 Hz, 1H), 7.26 (s, 1H), 7.21 (d, J=8.4 Hz, 1H), 7.13 (t, J=7.6 Hz, 1H), 4.62 (s, 1H), 4.15 (qd, J=9.6, 6.1 Hz, 2H), 3.54 (d, J=12.5 Hz, 1H), 2.93 (ddt, J=13.4, 10.0, 4.5 Hz, 2H), 2.38 (s, 1H), 2.13 (d, J=18.8 Hz, 1H), 2.07-1.94 (m, 1H), 1.81 (t, J=13.2 Hz, 1H), 1.56 (q, J=15.2, 13.9 Hz, 1H).

### Example-4: Synthesis of 5-(5-(2-(azetidin-3-yloxy)phenyl) isoxazol-3-ylamino) pyrazine-2-carbonitrile (Compound 1.4)

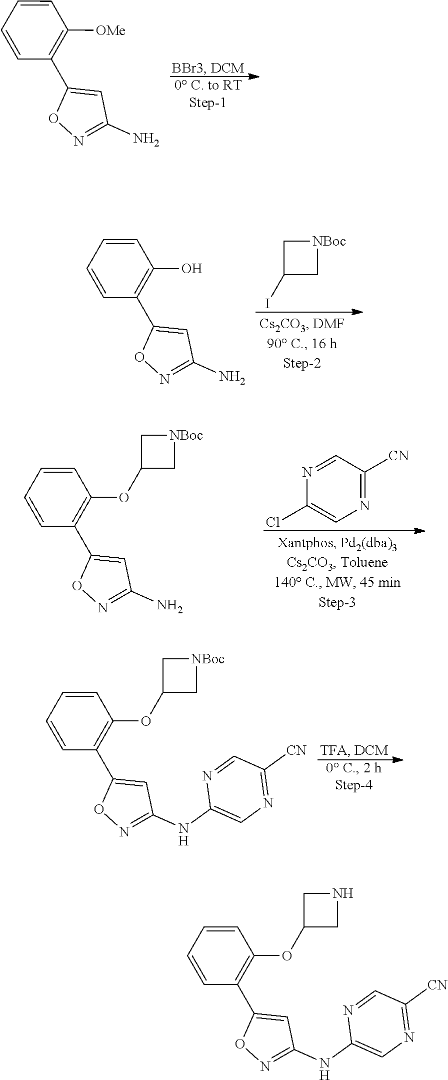

Step-1: Synthesis of 2-(3-aminoisoxazol-5-yl) phenol: Same as described in Compound 1.1

Step-2: Synthesis of tert-butyl 3-(2-(3-aminoisoxazol-5-yl) phenoxy) azetidine-1-carboxylate: To a solution of 2-(3-aminoisoxazol-5-yl) phenol (310 mg, 1.76 mmol, 1.0 eq) in DMF (5 mL) was added tert-butyl 3-iodoazetidine-1-carboxylate (498 mg, 1.76 mmol, 1.0 eq) and Cs₂CO₃ (803 mg, 2.46 mmol, 1.4 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (210 mg). LCMS: 332.3 [M+1]⁺

Step-3: Synthesis tert-butyl 3-(2-(3-(5-cyanopyrazin-2-ylamino) isoxazol-5-yl) phenoxy) azetidine-1-carboxylate: A solution of tert-butyl 3-(2-(3-aminoisoxazol-5-yl) phenoxy) azetidine-1-carboxylate (200 mg, 0.604 mmol, 1.0 eq) and 5-chloropyrazine-2-carbonitrile (101 mg, 0.725 mmol, 1.2 eq) in toluene (5 mL) was purged with N₂ for 5 minutes followed by addition of Cs₂CO₃ (590 mg, 1.81 mmol, 3.0 eq). The resultant reaction mixture was again purged for 5 minutes, then Xantphos (21 mg, 0.036 mmol, 0.06 eq) and Pd₂dba₃ (44 mg, 0.048 mmol, 0.08 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired compound (70 mg). LCMS: 435.3 [M+1]⁺

Step-4: Synthesis of 5-(5-(2-(azetidin-3-yloxy)phenyl) isoxazol-3-ylamino) pyrazine-2-carbonitrile: To a solution of tert-butyl 3-(2-(3-(5-cyanopyrazin-2-ylamino) isoxazol-5-yl) phenoxy) azetidine-1-carboxylate (70 mg, 0.161 mmol, 1.0 eq) in DCM (4.0 mL) TFA (0.4 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (40 mg, trifluoroacetate salt). LCMS: 335.3 [M+1]⁺ 99.73 @ 220 nm 99.93 @ 254 nm. ¹H NMR (400 MHZ, DMSO-d6, after D₂O addition) § 8.80 (s, 1H), 8.60 (s, 1H), 7.91 (dd, J=7.7, 1.7 Hz, 1H), 7.50 (t, J=7.7 Hz, 1H), 7.45 (s, 1H), 7.22 (t, J=7.6 Hz, 1H), 6.91 (d, J=8.3 Hz, 1H), 5.26 (h, J=6.0, 5.3 Hz, 1H), 4.56 (dd, J=12.3, 6.6 Hz, 2H), 4.10 (dd, J=12.6, 4.6 Hz, 2H).

### Example-5: Synthesis of 5-(5-(2-(3-aminopropoxy)phenyl) isoxazol-3-ylamino) pyrazine-2-carbonitrile (Compound 1.5)

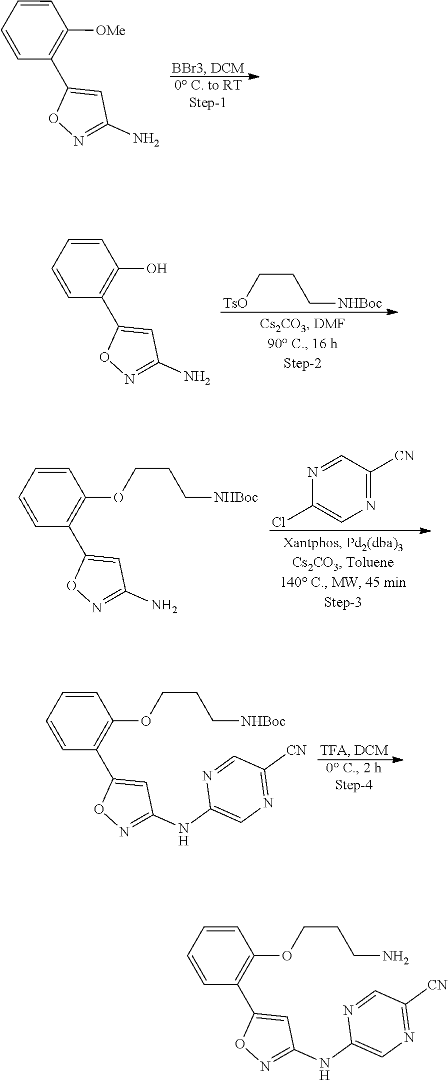

Step-1: Synthesis of 2-(3-aminoisoxazol-5-yl) phenol: Same as described in Compound 1.1

Step-2: Synthesis of tert-butyl 3-(2-(3-aminoisoxazol-5-yl) phenoxy) propylcarbamate: To a solution of 2-(3-aminoisoxazol-5-yl) phenol (270 mg, 1.53 mmol, 1.0 eq) in DMF (5 mL) was added 3-(tert-butoxycarbonylamino) propyl 4-methylbenzenesulfonate (synthesized as shown in WO 2020251971 A1) (505 mg, 1.53 mmol, 1.0 eq) and Cs₂CO₃ (699 mg, 2.15 mmol, 1.4 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (180 mg). LCMS: 334.3 [M+1]⁺

Step-3: Synthesis of tert-butyl 3-(2-(3-(5-cyanopyrazin-2-ylamino) isoxazol-5-yl) phenoxy) propylcarbamate: A solution of tert-butyl 3-(2-(3-aminoisoxazol-5-yl) phenoxy) propylcarbamate (150 mg, 0.45 mmol, 1.0 eq) and 5-chloropyrazine-2-carbonitrile (75 mg, 0.54 mmol, 1.2 eq) in toluene (3 mL) was purged with N₂ for 5 minutes followed by addition of Cs₂CO₃ (439 mg, 1.35 mmol, 3.0 eq). The resultant reaction mixture was again purged for 5 minutes, then Xantphos (16 mg, 0.027 mmol, 0.06 eq) and Pd₂dba₃ (33 mg, 0.036 mmol, 0.08 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate: hexane solvent system to obtain the desired compound (50 mg). LCMS: 437.3 [M+1]⁺

Step-4: Synthesis of 5-(5-(2-(3-aminopropoxy)phenyl) isoxazol-3-ylamino) pyrazine-2-carbonitrile: To a solution of tert-butyl 3-(2-(3-(5-cyanopyrazin-2-ylamino) isoxazol-5-yl) phenoxy) propylcarbamate (50 mg, 0.11 mmol, 1.0 eq) in DCM (3.0 mL) TFA (0.3 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (17 mg, formate salt). LCMS: 337.3 [M+1]⁺; 96.86 @ 220 nm 98.44 @ 254 nm. ¹H NMR (400 MHZ, Methanol-d4) δ 8.79 (s, 1H), 8.60 (s, 1H), 8.41 (s, 1H, formate), 7.87 (d, J=7.6 Hz, 1H), 7.51 (t, J=7.8 Hz, 1H), 7.33 (s, 1H), 7.22 (d, J=8.5 Hz, 1H), 7.14 (t, J=7.6 Hz, 1H), 4.24 (t, J=6.1 Hz, 2H), 3.07 (t, J=7.3 Hz, 2H), 2.14 (p, J=6.5 Hz, 2H).

### Example-6: Synthesis of 5-(5-(2-(4-aminobutoxy)phenyl) isoxazol-3-ylamino) pyrazine-2-carbonitrile (Compound 1.6)

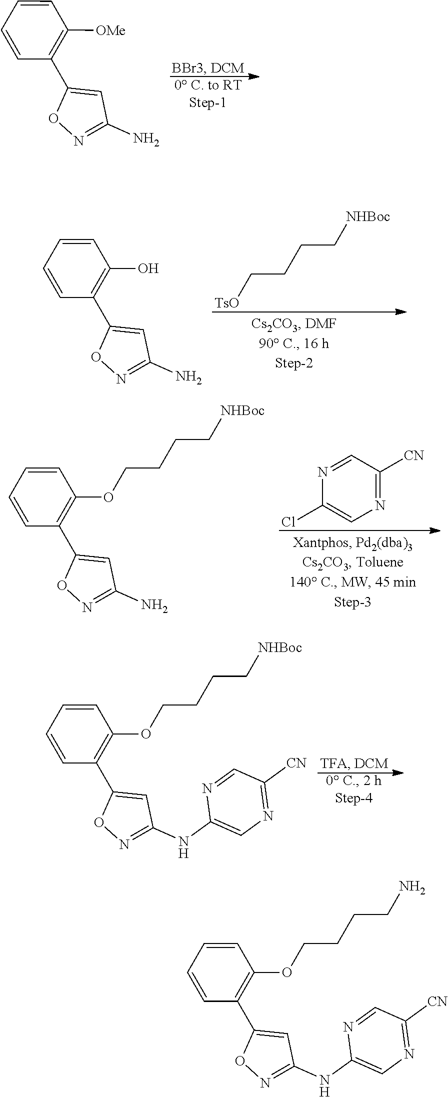

Step-1: Synthesis of 2-(3-aminoisoxazol-5-yl) phenol: Same as described in Compound 1.1

Step-2: Synthesis of tert-butyl 4-(2-(3-aminoisoxazol-5-yl) phenoxy)butylcarbamate: To a solution of 2-(3-aminoisoxazol-5-yl) phenol (205 mg, 1.16 mmol, 1.0 eq) in DMF (3 mL) was added 4-(tert-butoxycarbonylamino)butyl 4-methylbenzenesulfonate (synthesized as shown in WO 2021155321 A2) (400 mg, 1.16 mmol, 1.0 eq) and Cs₂CO₃ (531 mg, 1.63 mmol, 1.4 eq) in it. The resultant reaction mixture was stirred at 90° C. for 16 h. Product formation was confirmed by TLC and LCMS. After the completion of reaction, the reaction mixture was quenched with ice water (30 mL) and extracted with ethyl acetate (2× 20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (150 mg). LCMS: 348.3 [M+1]⁺

Step-3: Synthesis tert-butyl 4-(2-(3-(5-cyanopyrazin-2-ylamino) isoxazol-5-yl) phenoxy)butylcarbamate: A solution of tert-butyl 4-(2-(3-aminoisoxazol-5-yl) phenoxy)butylcarbamate (140 mg, 0.403 mmol, 1.0 eq) and 5-chloropyrazine-2-carbonitrile (67 mg, 0.483 mmol, 1.2 eq) in toluene (5 mL) was purged with N₂ for 5 minutes followed by addition of Cs₂CO₃ (393 mg, 1.21 mmol, 3.0 eq). The resultant reaction mixture was again purged for 5 minutes, then Xantphos (14.0 mg, 0.024 mmol, 0.06 eq) and Pd₂dba₃ (30 mg, 0.032 mmol, 0.08 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate: hexane solvent system to obtain the desired compound (90 mg). LCMS: 451.4 [M+1]⁺

Step-4: Synthesis of 5-(5-(2-(4-aminobutoxy)phenyl) isoxazol-3-ylamino) pyrazine-2-carbonitrile: To a solution of tert-butyl 4-(2-(3-(5-cyanopyrazin-2-ylamino) isoxazol-5-yl) phenoxy)butylcarbamate (90 mg, 0.20 mmol, 1.0 eq) in DCM (5.0 mL) TFA (0.3 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (29 mg, formate salt). LCMS: 351.4 [M+1]⁺95.84 @ 220 nm. ¹H NMR (400 MHZ, DMSO-d₆) δ 8.75 (s, 1H), 8.62 (s, 1H), 8.41 (s, 1H, formate), 7.86 (d, J=8.1 Hz, 1H), 7.50 (t, J=7.8 Hz, 1H), 7.32 (s, 1H), 7.22 (d, J=8.5 Hz, 1H), 7.12 (t, J=7.6 Hz, 1H), 4.17 (t, J=6.1 Hz, 2H), 2.86 (t, J=7.5 Hz, 2H), 1.91 (p, J=6.6 Hz, 2H), 1.77 (p, J=7.5 Hz, 2H).

### Example-7: Synthesis of 5-(5-(2-(2-aminoethoxy)phenyl) isoxazol-3-ylamino) pyrazine-2-carbonitrile (Compound 1.7)

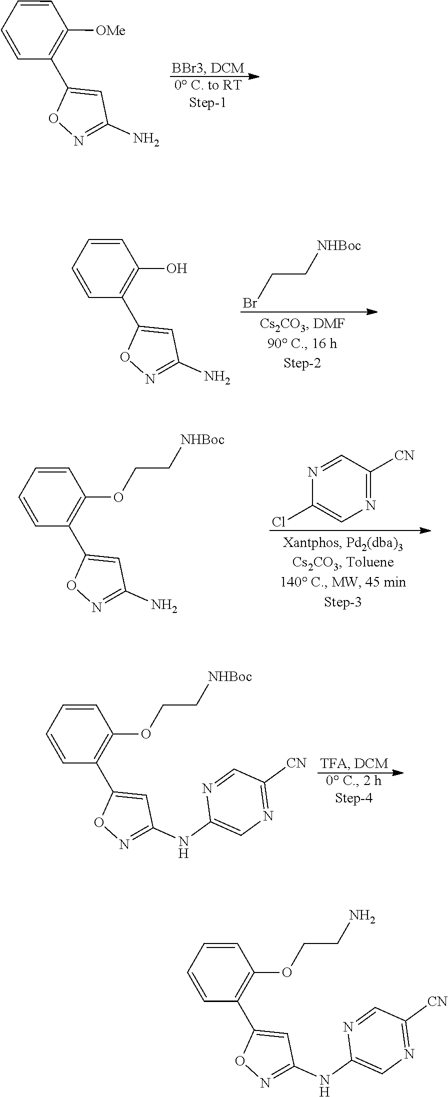

Step-1: Synthesis of 2-(3-aminoisoxazol-5-yl) phenol: Same as described in Compound 1.1

Step-2: Synthesis of tert-butyl 2-(2-(3-aminoisoxazol-5-yl) phenoxy)ethylcarbamate: To a solution of 2-(3-aminoisoxazol-5-yl) phenol (250 mg, 1.42 mmol, 1.0 eq) in DMF (3 mL) was added tert-butyl 2-bromoethylcarbamate (318 mg, 1.42 mmol, 1.0 eq) and Cs₂CO₃ (601 mg, 1.84 mmol, 1.3 eq) in it. The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (210 mg). LCMS: 320.4 [M+1]⁺

Step-3: Synthesis tert-butyl 2-(2-(3-(5-cyanopyrazin-2-ylamino) isoxazol-5-yl) phenoxy)ethylcarbamate: A solution of tert-butyl 2-(2-(3-aminoisoxazol-5-yl) phenoxy)ethylcarbamate (200 mg, 0.626 mmol, 1.0 eq) and 5-chloropyrazine-2-carbonitrile (105 mg, 0.75 mmol, 1.2 eq) in toluene (5 mL) was purged with N₂ for 5 minutes followed by addition of Cs₂CO₃ (612 mg, 1.88 mmol, 3.0 eq). The resultant reaction mixture was again purged for 5 minutes, then Xantphos (21 mg, 0.037 mmol, 0.06 eq) and Pd₂dba₃ (45 mg, 0.050 mmol, 0.08 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3× 15 mL). Collective organic layer was washed with brine (20 mL) and dried over anhydrous sodium sulfate and then concentrated under reduced pressure to get the crude. The crude was purified by combi flash chromatography by using 12 g RediSep® column and by eluting in ethyl acetate: hexane solvent system to obtain the desired compound (70 mg). LCMS: 423.3 [M+1]⁺

Step-4: Synthesis of 5-(5-(2-(2-aminoethoxy)phenyl) isoxazol-3-ylamino) pyrazine-2-carbonitrile: To a solution of tert-butyl 2-(2-(3-(5-cyanopyrazin-2-ylamino) isoxazol-5-yl) phenoxy)ethylcarbamate (70 mg, 0.17 mmol, 1.0 eq) in DCM (5.0 mL), TFA (0.4 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (20 mg, formate salt). LCMS: 323.3 [M+1]⁺; 99.96 @ 220 nm 99.97 @ 254 nm. ¹H NMR (400 MHZ, DMSO-d6, after D2O addition) δ 8.78 (s, 1H), 8.60 (s, 1H, formate), 8.26 (s, 1H), 7.87 (d, J=7.9 Hz, 1H), 7.52 (t, J=7.9 Hz, 1H), 7.36 (s, 1H), 7.24 (d, J=8.5 Hz, 1H), 7.17 (t, J=7.6 Hz, 1H), 4.31 (t, J=5.4 Hz, 2H), 3.29 (t, J=5.3 Hz, 2H).

### Example-8: Synthesis of 5-(5-(2-((4-methylmorpholin-2-yl) methoxy)phenyl) isoxazol-3-ylamino) pyrazine-2-carbonitrile (Compound 1.8)

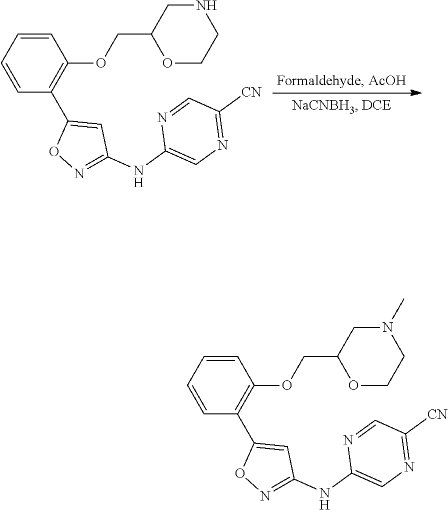 [SMILES: N#Cc1cnc(Nc2cc(-c3ccccc3OCC3CNCCO3)on2)cn1 ; CN1CCOC(COc2ccccc2-c2cc(Nc3cnc(C#N)cn3)no2)C1]

Step-1: Synthesis of 5-(5-(2-((4-methylmorpholin-2-yl) methoxy)phenyl) isoxazol-3-ylamino) pyrazine-2-carbonitrile: To a stirred solution of 5-(5-(2-(morpholin-2-ylmethoxy)phenyl) isoxazol-3-ylamino) pyrazine-2-carbonitrile (70 mg, 0.185 mmol, 1 eq) in dichloroethane was added acetic acid (0.1 mL) and formaldehyde (37% solution) (0.1 mL, 1.11 mmol, 6 eq). The resultant reaction mixture was stirred at room temperature for 1 h. Then the reaction mixture was cooled to 0° C. and sodium cyanoborohydride (58 mg, 0.925 mmol, 5 eq) was added to it. The reaction mixture was stirred for 3 h at RT. Product formation was confirmed by TLC and LCMS. The reaction mixture was quenched using saturated sodium bicarbonate (2×5 mL) and the organic layer was separated and concentrated in vacuum to provide the crude. The crude was purified by R-HPLC to yield desired compound (8 mg, formate salt). LCMS: 393.5 [M+1]⁺ 97.91 @ 220 nm; 99.10 @ 254 nm. ¹H NMR (400 MHZ, DMSO-d6) δ 11.32 (br. s., 1H, NH), 8.83-8.79 (m, 1H), 8.63 (s, 1H), 8.15 (s, 1H, formate CH), 7.88 (dd, J=7.87, 1.19 Hz, 1H), 7.53-7.47 (m, 1H), 7.39 (s, 1H), 7.23 (d, J=8.58 Hz, 1H), 7.13 (t, J=7.63 Hz, 1H), 4.16 (d, J=5.25 Hz, 2H), 3.99-3.91 (m, 2H), 3.87 (d, J=11.44 Hz, 2H), 3.66-3.55 (m, 1H), 2.18 (s, 3H), 2.06-1.89 (m, 2H).

### Example-9: Synthesis of 5-(3-(2-methoxy-4-(1,2,3,6-tetrahydropyridin-4-yl)phenyl) isoxazol-5-ylamino) pyrazine-2-carbonitrile (Compound 1.9)

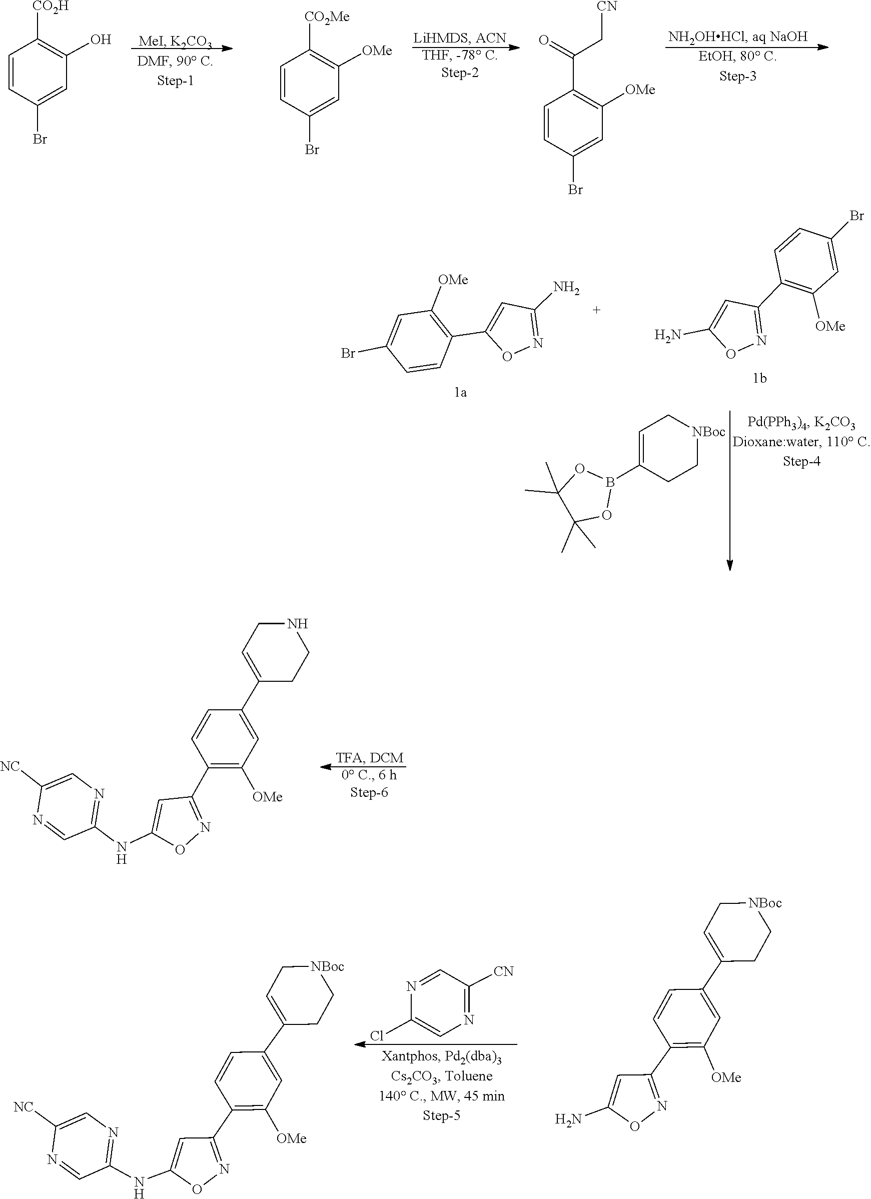

Step 1: Synthesis of methyl 4-bromo-2-methoxybenzoate: Synthesis was performed as shown in WO 2006044775 A2.

Step-2: Synthesis of 3-(4-bromo-2-methoxyphenyl)-3-oxopropanenitrile: To a solution of methyl 4-bromo-2-methoxybenzoate (5.0 g, 20.4 mmol, 1.0 eq) in THF (50 mL) was added 1.0 M solution of LiHMDS in THF (30.6 mL, 30.6 mmol, 1.5 eq) dropwise at −78° C. The resultant reaction mixture was stirred at the same temperature for 2 hours. Then acetonitrile (15 mL) was added dropwise to the reaction mixture and stirred for 1 hour. The product formation was confirmed by TLC and LCMS. After the completion of reaction, reaction mixture was quenched with saturated ammonium chloride solution (100 mL) and extracted with ethyl acetate (3×50 mL). The organic layer was separated and washed with brine (50 mL), dried over anhydrous Na₂SO₄ and concentrated under reduced pressure to obtain the desired compound (4.5 g). LCMS: 254.3 [M+1]⁺

Step-3: Synthesis of 3-(4-bromo-2-methoxyphenyl) isoxazol-5-amine (1b) and 5-(4-bromo-2-methoxyphenyl) isoxazol-3-amine (1a): To a solution of 3-(4-bromo-2-methoxyphenyl)-3-oxopropanenitrile (2.0 g, 7.87 mmol, 1.0 eq) in EtOH: H₂O (20 mL: 8 mL), hydroxyl amine hydrochloride (602 mg, 8.66 mmol, 1.1 eq) and sodium hydroxide (346 mg, 8.66 mmol, 1.1 eq) was added and stirred at 80° C. for 16 h. The progress of reaction was monitored by TLC and LCMS. After the completion of reaction, it was quenched with saturated sodium bicarbonate solution (50 mL) and extracted with ethyl acetate (3×50 mL). The organic layer was separated and washed with brine (50 mL), dried over anhydrous Na₂SO₄ and concentrated under reduced pressure to obtain the crude. The crude was purified by flash chromatography in ethyl acetate: hexane solvent system to obtain the desired compounds 1a (1.2 g) and 1b (0.65 g). LCMS: 1a: 269.2 [M]⁺; 271.2 [M+2]⁺; 1b: 269.3 [M]⁺; 271.3 [M+2]⁺

Step-4: Synthesis of tert-butyl 4-(4-(5-aminoisoxazol-3-yl)-3-methoxyphenyl)-5,6-dihydropyridine-1 (2H)-carboxylate: A solution of 3-(4-bromo-2-methoxyphenyl) isoxazol-5-amine (1.2 g, 4.46 mmol, 1.0 eq) and tert-butyl 4-(4,4,5,5-tetramethyl-1,3,2-dioxaborolan-2-yl)-5,6-dihydropyridine-1 (2H)-carboxylate (1.52 g, 4.91 mmol, 1.1 eq) in dioxane: H₂O (10 mL: 2.0 mL) was purged with N₂ for 5 minutes. K₂CO₃ (1.54 g, 11.1 mmol, 2.5 eq) was added to the reaction mixture and purged again for 5 minutes followed by addition of tetrakis(triphenylphosphine) palladium (0) (515 mg, 0.445 mmol, 0.1 eq) and the reaction mixture was stirred at 110° C. for 16 h. Product formation was confirmed by TLC and LCMS. The reaction mixture was quenched with ice water (50 mL) and extracted with ethyl acetate (3×30 mL). The organic layer was washed with brine (30 mL) and dried over Na₂SO₄ and then concentrated under reduced pressure to get the crude. The crude was purified by flash chromatography by using 14 g RediSep® column in ethyl acetate: hexane solvent system to obtain the desired compound (500 mg). LCMS: 372.4 [M+1]⁺

Step-5: Synthesis of tert-butyl 4-(4-(5-((5-cyanopyrazin-2-ylamino) isoxazol-3-yl)-3-methoxyphenyl)-5,6-dihydropyridine-1 (2H)-carboxylate: A solution of tert-butyl 4-(4-(5-aminoisoxazol-3-yl)-3-methoxyphenyl)-5,6-dihydropyridine-1 (2H)-carboxylate (230 mg, 0.619 mmol, 1.0 eq) and 5-chloropyrazine-2-carbonitrile (104 mg, 0.743 mmol, 1.2 eq) in DMF (5.0 mL) was purged with N₂ for 5 minutes followed by addition of Cs₂CO₃ (605 mg, 1.86 mmol, 3.0 eq). The resultant reaction mixture was again purged for 5 minutes and then Xantphos (7.1 mg, 0.012 mmol, 0.02 eq) and Pd₂dba₃ (28 mg, 0.031 mmol, 0.05 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate: hexane solvent system to obtain the desired compound (120 mg). LCMS: 475.3 [M+1]⁺

Step-6: Synthesis of 5-(3-(2-methoxy-4-(1,2,3,6-tetrahydropyridin-4-yl)phenyl) isoxazol-5-ylamino) pyrazine-2-carbonitrile: To a solution of tert-butyl 4-(4-(5-((5-cyanopyrazin-2-ylamino) isoxazol-3-yl)-3-methoxyphenyl)-5,6-dihydropyridine-1 (2H)-carboxylate (120 mg, 0.253 mmol, 1 eq) in DCM (3 mL), TFA (0.096 mL, 1.26 mmol, 5 eq) was added at 0° C. and kept for stirring for 6 h. The reaction mixture was concentrated under reduced pressure to get the crude. The crude was purified by R-HPLC to yield desired compound (15 mg, Trifluoroacetate salt). LCMS: 375.5 [M+1]⁺97.1% @ 220 nm, 98.88 @ 254 nm. ¹H NMR (400 MHZ, DMSO-d6 after D₂O addition): δ 8.80 (d, J=1.4 Hz, 1H), 8.39 (d, J=1.4 Hz, 1H), 7.72 (d, J=7.9 Hz, 1H), 7.21-7.14 (m, 2H), 6.83 (s, 1H), 6.33 (s, 1H), 3.90 (s, 3H), 3.87 (s, 2H), 3.33 (t, J=6.1 Hz, 2H), 2.72 (s, 2H).

### Example-10: Synthesis of 5-(5-(2-(1-methylazetidin-3-yloxy)phenyl) isoxazol-3-ylamino) pyrazine-2-carbonitrile (Compound 1.10)

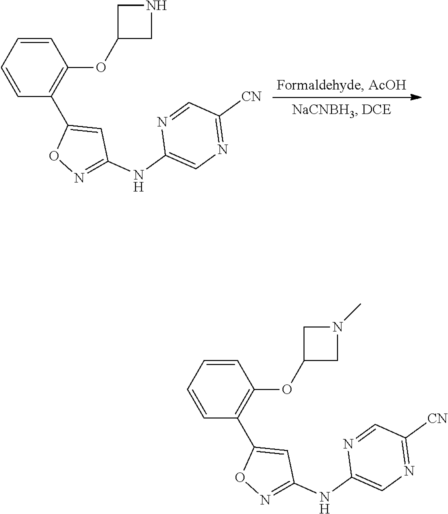 [SMILES: N#Cc1cnc(Nc2cc(-c3ccccc3OC3CNC3)on2)cn1 ; CN1CC(Oc2ccccc2-c2cc(Nc3cnc(C#N)cn3)no2)C1]

Step-1: Synthesis of 5-(5-(2-(1-methylazetidin-3-yloxy)phenyl) isoxazol-3-ylamino) pyrazine-2-carbonitrile: To a stirred solution of 5-(5-(2-(azetidin-3-yloxy)phenyl) isoxazol-3-ylamino) pyrazine-2-carbonitrile (70 mg, 0.209 mmol, 1 eq) in dichloroethane was added acetic acid (0.1 mL) and formaldehyde (37% solution) (0.1 mL, 1.05 mmol, 5 eq). The resultant reaction mixture was stirred at room temperature for 1 h. Then the reaction mixture was cooled to 0° C. and sodium cyanoborohydride (66 mg, 1.05 mmol, 5 eq) was added to the reaction mixture. The reaction mixture was stirred for 3 h at RT. Product formation was confirmed by TLC and LCMS. The reaction mixture was quenched using saturated sodium bicarbonate (2×5 mL) and the organic layer as separated and concentrated in vacuumto provide the crude. The crude was purified by R-HPLC to yield desired compound (10 mg, formate salt). LCMS: 349.5 [M+1]⁺99.39 @ 220 nm, 99.27 @ 254 nm. ¹H NMR (400 MHZ, DMSO-d6 after D₂O addition) δ 8.79 (s, 1H), 8.65 (s, 1H), 8.17 (s, 1H, formate CH), 7.89 (d, J=7.9 Hz, 1H), 7.47 (t, J=7.8 Hz, 1H), 7.39 (s, 1H), 7.16 (t, J=7.6 Hz, 1H), 6.95 (d, J=8.4 Hz, 1H), 5.02 (p, J=5.5 Hz, 1H), 4.00 (dd, J=9.1, 5.9 Hz, 2H), 3.36 (dd, J=9.2, 4.9 Hz, 2H), 2.46 (s, 3H).

### Example-11: Synthesis of 5-(5-(2-methoxyphenyl) isoxazol-3-ylamino) pyrazine-2-carbonitrile (Compound 1.11)

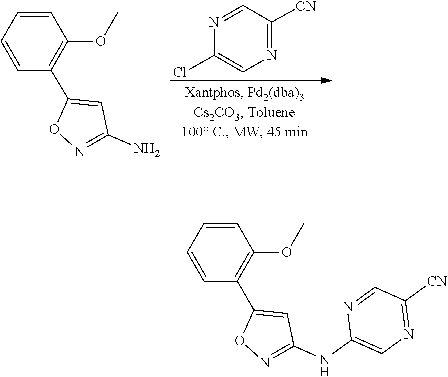 [SMILES: COc1ccccc1-c1cc(N)no1 ; N#Cc1cnc(Cl)cn1 ; COc1ccccc1-c1cc(Nc2cnc(C#N)cn2)no1]

Synthesis of 5-(5-(2-methoxyphenyl) isoxazol-3-ylamino) pyrazine-2-carbonitrile: A solution of 5-(2-methoxyphenyl) isoxazol-3-amine (200 mg, 1.05 mmol, 1.0 eq) and 5-chloropyrazine-2-carbonitrile (147 mg, 1.05 mmol, 1.0 eq) in toluene (5 mL) was purged with nitrogen for 5 minutes followed by addition of Cs₂CO₃ (1.03 mg, 3.15 mmol, 3.0 eq). The resultant reaction mixture was again purged for 5 minutes, then Xantphos (12.0 mg, 0.021 mmol, 0.02 eq) and Pd₂dba₃ (19 mg, 0.021 mmol, 0.02 eq) was added and the reaction mixture was subjected to microwave for 45 min at 100° C. Product formation was confirmed by TLC and LCMS. After the completion of reaction, the reaction mixture was quenched with ice water (10 mL) and extracted with ethyl acetate (2×20 mL). Collective organic layer was washed with brine (20 mL) and dried over anhydrous sodium sulfate and then concentrated under reduced pressure to get the crude. The crude was triturated with diethyl ether (2×5 mL) which provided the desired compound (12 mg) and did not require any further purification. LCMS: 294.3 [M+1]⁺97.31 @ 220 nm 98.2 @ 254 nm. ¹H NMR (400 MHZ, DMSO-d6, after D2O addition) δ 8.75 (s, 1H), 8.62 (s, 1H), 7.84 (d, J=7.7 Hz, 1H), 7.51 (t, J=7.9 Hz, 1H), 7.27-7.17 (m, 2H), 7.12 (t, J=7.5 Hz, 1H), 3.93 (s, 3H).

### Example-12: Synthesis of 5-(5-(2-(1-(4-methylpentan-2-yl) azetidin-3-yloxy)phenyl) isoxazol-3-ylamino) pyrazine-2-carbonitrile (Compound 1.12)

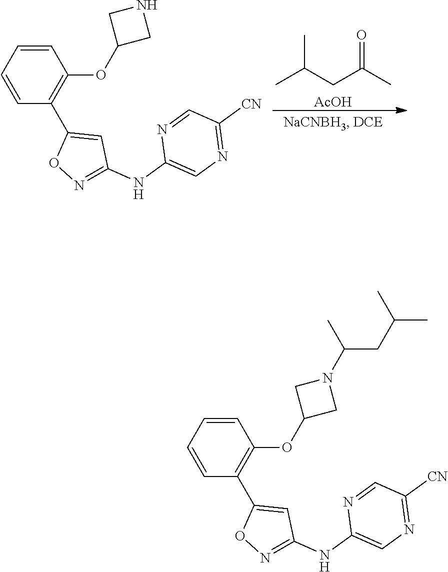

Step-1: Synthesis of 5-(5-(2-(1-(4-methylpentan-2-yl) azetidin-3-yloxy)phenyl) isoxazol-3-ylamino) pyrazine-2-carbonitrile: To a stirred solution of 5-(5-(2-(azetidin-3-yloxy)phenyl) isoxazol-3-ylamino) pyrazine-2-carbonitrile (70 mg, 0.209 mmol, 1 eq) in dichloroethane was added acetic acid (0.1 mL) and 4-methylpentan-2-one (105 mg, 1.05 mmol, 5 eq). The resultant reaction mixture was stirred at room temperature for 1 h. Then the reaction mixture was cooled to 0° C. and sodium cyanoborohydride (66 mg, 1.05 mmol, 5 eq) was added to the reaction mixture. The reaction mixture was stirred for 3 h at RT. Product formation was confirmed by TLC and LCMS. The reaction mixture was quenched using saturated sodium bicarbonate (2×5 mL) and the organic layer as separated and concentrated in vacuum to provide the crude. The crude was purified by R-HPLC to yield desired compound (23 mg, formate salt). LCMS: 419.2 [M+1]⁺ 96.82 @ 220 nm 97.95 @ 254 nm. ¹H NMR (400 MHZ, DMSO-d6 after D₂O addition) δ 8.73 (s, 1H), 8.66 (s, 1H), 8.19 (s, 1H, formate CH), 7.88 (dd, J=7.7, 1.7 Hz, 1H), 7.53-7.42 (m, 1H), 7.35 (s, 1H), 7.15 (t, J=7.6 Hz, 1H), 6.98 (d, J=8.3 Hz, 1H), 4.96 (p, J=5.4 Hz, 1H), 3.91 (q, J=7.9 Hz, 2H), 3.20 (q, J=6.5 Hz, 2H), 2.43 (q, J=5.3, 4.0 Hz, 1H), 1.57 (dq, J=11.8, 5.8 Hz, 1H), 1.21 (ddd, J=13.2, 9.8, 3.6 Hz, 1H), 1.02 (ddd, J=13.6, 9.7, 4.3 Hz, 1H), 0.90 (d, J=5.9 Hz, 3H), 0.87 (d, J=6.6 Hz, 3H), 0.81 (d, J=6.4 Hz, 3H).

### Example-13: Synthesis of 5-(5-(2-(1-isopropylazetidin-3-yloxy)phenyl) isoxazol-3-ylamino) pyrazine-2-carbonitrile (Compound 1.13)

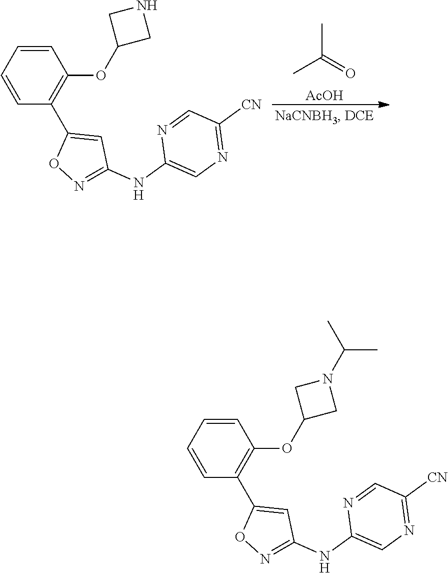

Step-1: Synthesis of 5-(5-(2-(1-isopropylazetidin-3-yloxy)phenyl) isoxazol-3-ylamino) pyrazine-2-carbonitrile: To a stirred solution of 5-(5-(2-(azetidin-3-yloxy)phenyl) isoxazol-3-ylamino) pyrazine-2-carbonitrile (70 mg, 0.209 mmol, 1 eq) in dichloroethane was added acetic acid (0.1 mL) and propan-2-one (63 mg, 1.05 mmol, 5 eq). The resultant reaction mixture was stirred at room temperature for 1 h. Then the reaction mixture was cooled to 0° C. and sodium cyanoborohydride (66 mg, 1.05 mmol, 5 eq) was added to the reaction mixture. The reaction mixture was stirred for 3 h at RT. Product formation was confirmed by TLC and LCMS. The reaction mixture was quenched using saturated sodium bicarbonate (2×5 mL) and the organic layer was separated and concentrated in vacuum to provide the crude. The crude was purified by R-HPLC to yield desired compound (7 mg, formate salt). LCMS: 377.3 [M+1]⁺98.99 @ 220 nm 99.41 @ 254 nm. ¹H NMR (400 MHZ, DMSO-d6 after D₂O addition) δ 8.74 (s, 1H), 8.67 (s, 1H), 8.21 (s, 1H, formate CH), 7.87 (d, J=7.7 Hz, 1H), 7.47 (t, J=8.0 Hz, 1H), 7.31 (s, 1H), 7.15 (t, J=7.5 Hz, 1H), 6.97 (d, J=8.4 Hz, 1H), 4.97 (q, J=5.6 Hz, 1H), 3.94 (t, J=7.2 Hz, 2H), 3.27 (dd, J=9.2, 5.0 Hz, 2H), 2.58 (d, J=6.4 Hz, 1H), 0.94 (d, J=6.2 Hz, 6H).

### Example-14: Synthesis of 5-(5-(2-(pyrrolidin-3-yloxy)phenyl) isoxazol-3-ylamino) pyrazine-2-carbonitrile (Compound 1.14)

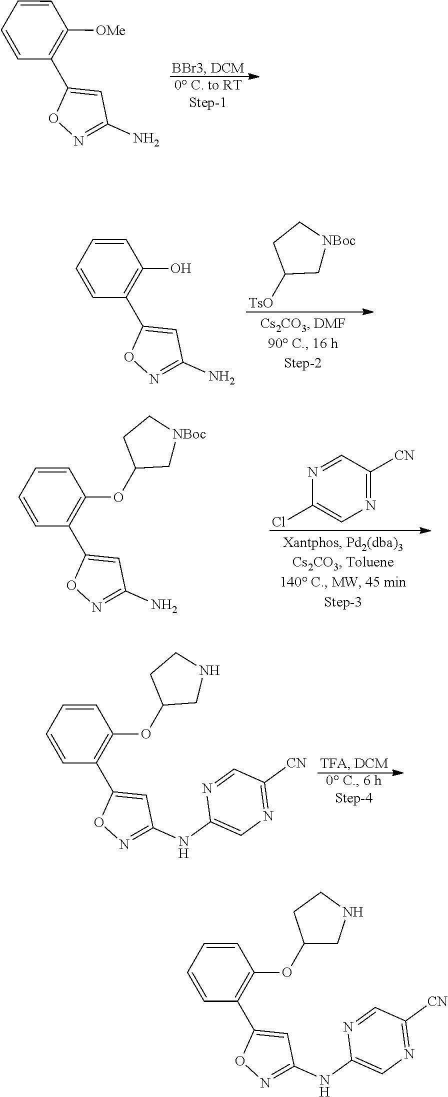

Step-1: Synthesis of 2-(3-aminoisoxazol-5-yl) phenol: Same as described in Compound 1.1

Step-2: Synthesis of tert-butyl 3-(2-(3-aminoisoxazol-5-yl) phenoxy) pyrrolidine-1-carboxylate: To a solution of 2-(3-aminoisoxazol-5-yl) phenol (300 mg, 1.7 mmol, 1.0 eq) in DMF was added tert-butyl 3-(tosyloxy) pyrrolidine-1-carboxylate (synthesized as described in J. Med. Chem, 2020, 63 (19), 11054-84) (698 mg, 2.04 mmol, 1.2 eq) and Cs₂CO₃ (1.11 g, 3.41 mmol, 2 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 4 g RediSep® column in ethyl acetate: hexane solvent system to obtain the desired compound (200 mg) as a colorless liquid. LCMS: 346.3 [M+1]⁺

Step-3: Synthesis of tert-butyl 3-(2-(3-(5-cyanopyrazin-2-ylamino) isoxazol-5-yl) phenoxy) pyrrolidine-1-carboxylate: A solution of tert-butyl 3-(2-(3-aminoisoxazol-5-yl) phenoxy) pyrrolidine-1-carboxylate (200 mg, 0.579 mmol, 1.0 eq) and 5-chloropyrazine-2-carbonitrile (97 mg, 0.695 mmol, 1.2 eq) in toluene (3.0 mL) was purged with N₂ for 5 minutes followed by addition of Cs₂CO₃ (566 mg, 1.74 mmol, 3.0 eq). The resultant reaction mixture was again purged for 5 minutes, then Xantphos (6.7 mg, 0.012 mmol, 0.02 eq) and Pd₂dba₃ (26 mg, 0.029 mmol, 0.05 eq) was added and the reaction mixture was subjected to microwave for 45 minutes at 140° C. Product formation was confirmed by TLC and LCMS. After the completion of reaction, the reaction mixture was quenched with ice water (20 mL) and extracted with ethyl acetate (3× 15 mL). Collective organic layer was washed with brine (20 mL) and dried over anhydrous sodium sulfate and then concentrated under reduced pressure to get the crude. The crude was purified by flash chromatography by using 4 g RediSep® column in ethyl acetate: hexane solvent system to obtain the desired compound (50 mg). LCMS: 449.2 [M+1]⁺

Step-4: Synthesis of 5-(5-(2-(pyrrolidin-3-yloxy)phenyl) isoxazol-3-ylamino) pyrazine-2-carbonitrile: To a solution of tert-butyl 3-(2-(3-(5-cyanopyrazin-2-ylamino) isoxazol-5-yl) phenoxy) pyrrolidine-1-carboxylate (50 mg, 0.111 mmol, 1 eq) in DCM (4 mL), TFA (0.1 mL) was added at 0° C. and kept for stirring for 6 h. The reaction mixture was concentrated under reduced pressure to get the crude. The crude was treated with 5 mL ethyl acetate which led to precipitation of a product (8 mg, trifluoroacetate salt). This product was filtered, characterized by NMR, MS to confirm the formation of desired compound. LCMS: 349.3 [M+1]⁺UPLC: 97.13 @ 220 nm 97.97 @ 254 nm; ¹H NMR (400 MHZ, Methanol-d4) δ 8.65 (d, J=1.5 Hz, 1H), 8.60 (s, 1H), 7.93 (dd, J=7.7, 1.7 Hz, 1H), 7.55-7.49 (m, 1H), 7.33 (s, 1H), 7.23-7.16 (m, 2H), 5.40 (s, 1H), 3.68-3.56 (m, 4H), 2.53-2.39 (m, 2H).

### Example-15: Synthesis of 5-(5-(2-((1s,4s)-4-aminocyclohexyloxy)phenyl) isoxazol-3-ylamino) pyrazine-2-carbonitrile (Compound 1.15)

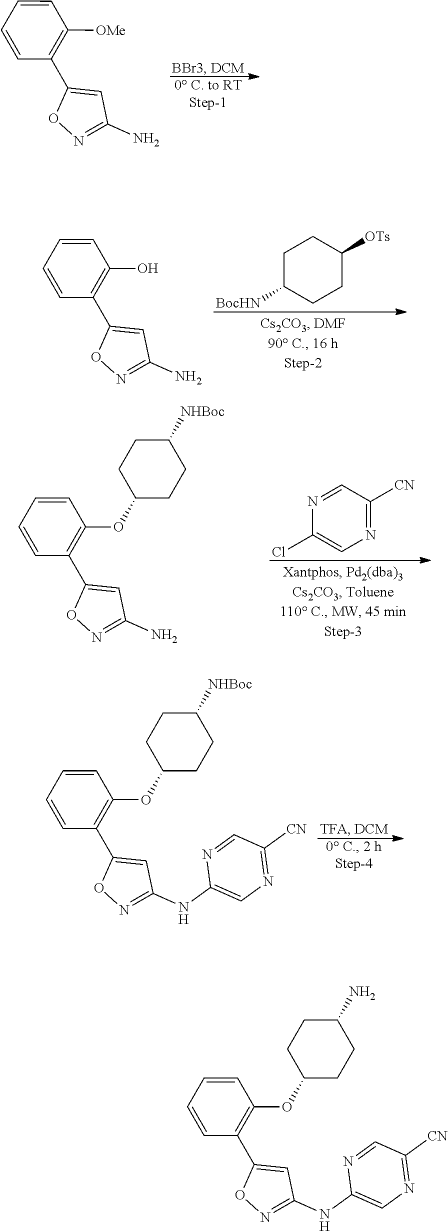

Step-1: Synthesis of 2-(3-aminoisoxazol-5-yl) phenol: Same as described in Compound 1.1

Step-2: Synthesis of tert-butyl (1s,4s)-4-(2-(3-aminoisoxazol-5-yl) phenoxy)cyclohexylcarbamate: To a solution of 2-(3-aminoisoxazol-5-yl) phenol (0.476 g, 2.70 mmol, 1.0 eq) in DMF (10 mL) was added (1r,4r)-4-(tert-butoxycarbonylamino)cyclohexyl 4-methylbenzenesulfonate (synthesized as described in WO2021080015 A1) (0.998 g, 2.70 mmol, 1.0 eq) and Cs₂CO₃ (1.76 g, 5.40 mmol, 2.0 eq). The resultant reaction mixture was stirred at 90° C. for 16 h. Product formation was confirmed by TLC and LCMS. After the completion of reaction, the reaction mixture was quenched with ice water (100 mL) and extracted with ethyl acetate (2×50 mL). Collective organic layer was washed with brine (50 mL) and dried over anhydrous sodium sulfate and then concentrated under reduced pressure to get the crude. The crude was purified by flash chromatography by using 24 g RediSep® column in ethyl acetate: hexane solvent system to obtain the desired product (320 mg). LCMS: 374.3 [M+1]⁺

Step-3: Synthesis of tert-butyl (1s,4s)-4-(2-(3-(5-cyanopyrazin-2-ylamino) isoxazol-5-yl) phenoxy)cyclohexylcarbamate: A solution of tert-butyl (1s,4s)-4-(2-(3-aminoisoxazol-5-yl) phenoxy)cyclohexylcarbamate (150 mg, 0.40 mmol, 1.0 eq) and 5-chloropyrazine-2-carbonitrile (56 mg, 0.40 mmol, 1.0 eq) in toluene (5 mL) was purged with N₂ for 5 minutes followed by addition of Cs₂CO₃ (392 mg, 1.20 mmol, 3.0 eq). The resultant reaction mixture was again purged for 5 minutes, then Xantphos (14 mg, 0.024 mmol, 0.06 eq) and Pd₂dba₃ (29 mg, 0.032 mmol, 0.08 eq) was added and the reaction mixture was subjected to microwave for 45 minutes at 110° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by flash chromatography by using 12 g RediSep® column and by eluting in ethyl acetate: hexane solvent system to obtain the desired compound (70 mg). LCMS: 477.2 [M+1]⁺

Step-4: Synthesis of 5-(5-(2-((1s,4s)-4-aminocyclohexyloxy)phenyl) isoxazol-3-ylamino) pyrazine-2-carbonitrile: To a solution of tert-butyl (1s,4s)-4-(2-(3-(5-cyanopyrazin-2-ylamino) isoxazol-5-yl) phenoxy)cyclohexylcarbamate (70 mg, 0.14 mmol, 1.0 eq) in DCM (5.0 mL), TFA (0.4 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (20 mg, formate salt). LCMS: 377.3 [M+1]⁺ 99.99 @ 220 nm; 99.94 @ 254 nm. ¹H NMR (400 MHz, DMSO-d6, after D2O addition) δ 8.67 (s, 1H), 8.54 (s, 1H), 8.39 (s, 1H, formate CH), 7.87 (d, J=7.6 Hz, 1H), 7.51-7.42 (m, 2H), 7.21 (d, J=8.5 Hz, 1H), 7.09 (t, J=7.6 Hz, 1H), 4.81 (s, 1H), 3.12 (dt, J=13.1, 6.2 Hz, 1H), 2.12 (d, J=12.6 Hz, 2H), 1.86 (dd, J=10.1, 4.7 Hz, 2H), 1.72 (h, J=12.6, 11.5 Hz, 4H).

### Example-16: Synthesis of 5-(5-(2-((1-methylpiperidin-3-yl) methoxy)phenyl) isoxazol-3-ylamino) pyrazine-2-carbonitrile (Compound 1.16)

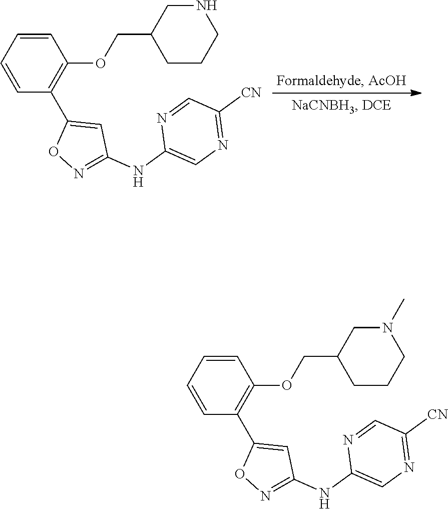 [SMILES: N#Cc1cnc(Nc2cc(-c3ccccc3OCC3CCCNC3)on2)cn1 ; CN1CCCC(COc2ccccc2-c2cc(Nc3cnc(C#N)cn3)no2)C1]

Step-1: Synthesis of 5-(5-(2-((1-methylpiperidin-3-yl) methoxy)phenyl) isoxazol-3-ylamino) pyrazine-2-carbonitrile: To a stirred solution of 5-(5-(2-(piperidin-3-ylmethoxy)phenyl) isoxazol-3-ylamino) pyrazine-2-carbonitrile (70 mg, 0.185 mmol, 1 eq) in dichloroethane (5 mL) was added acetic acid (0.1 mL) and formaldehyde (37% solution) (0.075 ml, 0.929 mmol, 5 eq). The resultant reaction mixture was stirred at room temperature for 1 h. Then the reaction mixture was cooled to 0° C. and sodium cyanoborohydride (58 mg, 0.929 mmol, 5 eq) was added to the reaction mixture. The reaction mixture was stirred for 3 h at RT. Product formation was confirmed by TLC and LCMS. The reaction mixture was quenched using saturated sodium bicarbonate (2×5 mL) and the organic layer as separated and concentrated in vacuum to provide the crude. The crude was purified by R-HPLC to yield desired compound (10 mg, trifluoroacetate salt). LCMS: 391.5 [M+1]⁺99.59 @ 220 nm; 99.78 @ 254 nm. ¹H NMR (400 MHZ, Methanol-d4) δ 8.65 (d, J=15.0 Hz, 2H), 7.86 (d, J=7.7 Hz, 1H), 7.50 (t, J=7.9 Hz, 1H), 7.27-7.06 (m, 3H), 4.16 (p, J=9.9 Hz, 2H), 3.71-3.49 (m, 2H), 2.95 (dd, J=12.5, 7.8 Hz, 2H), 2.91 (s, 3H), 2.44 (s, 1H), 2.13 (t, J=15.8 Hz, 2H), 1.92-1.78 (m, 1H), 1.51 (tt, J=13.9, 7.0 Hz, 1H).

### Example-17: Synthesis of 5-(5-(2-(azetidin-3-ylmethoxy)phenyl) isoxazol-3-ylamino) pyrazine-2-carbonitrile (Compound 1.17)

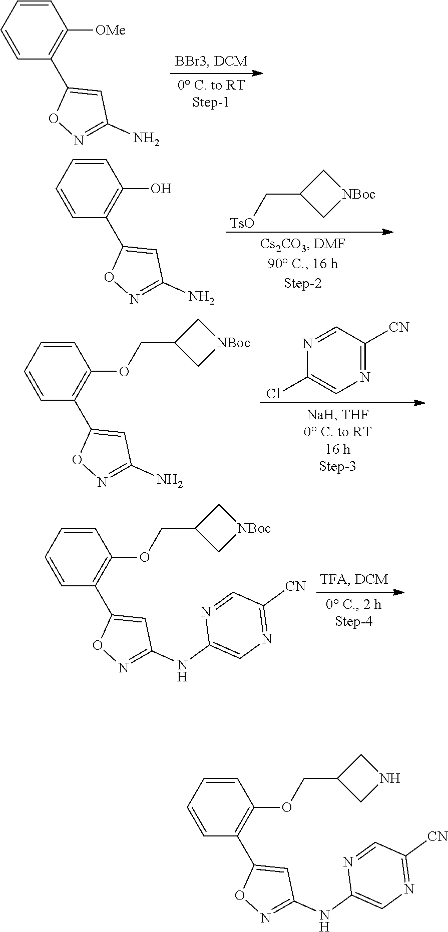

Step-1: Synthesis of 2-(3-aminoisoxazol-5-yl) phenol: Same as described in Compound 1.1

Step-2: Synthesis of tert-butyl 3-((2-(3-aminoisoxazol-5-yl) phenoxy)methyl) azetidine-1-carboxylate: To a solution of 2-(3-aminoisoxazol-5-yl) phenol (1.0 g, 5.68 mmol, 1.0 eq) in DMF (25 mL) was added tert-butyl 3-(tosyloxymethyl) azetidine-1-carboxylate (synthesized as described in EP 2676965 A1) (2.33 g, 6.81 mmol, 1.2 eq) and Cs₂CO₃ (3.7 g, 11.35 mmol, 2.0 eq) in it. The resultant reaction mixture was stirred at 90° C.° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column by using ethyl acetate: hexane solvent system to obtain the desired product (800 mg). LCMS: 346.4 [M+1]⁺

Step-3: Synthesis tert-butyl 3-((2-(3-(5-cyanopyrazin-2-ylamino) isoxazol-5-yl) phenoxy)methyl) azetidine-1-carboxylate: To a solution of tert-butyl 3-((2-(3-aminoisoxazol-5-yl) phenoxy)methyl) azetidine-1-carboxylate (0.5 g, 1.45 mmol, 1.0 eq) in THF (10.0 mL) at 0° C. was added sodium hydride (60% dispersion in mineral oil) (0.173 g, 4.34 mmol, 3.0 eq) portion wise over the period of 10 min. The reaction mixture was then stirred for 30 min at 0° C. To this reaction mixture was added 5-chloropyrazine-2-carbonitrile (0.241 g, 1.74 mmol, 1.2 eq) and it was stirred at RT for 16 h. Product formation was confirmed by TLC and LCMS. The reaction mixture was quenched using ice-cold water (30 mL) and extracted using ethyl acetate (2×50 mL). The combined organic layer was washed with brine (30 mL), dried over anhydrous sodium sulfate and concentrated under reduced pressure to get the crude. The crude was purified by flash chromatography by using 12 g RediSep® column in ethyl acetate: hexane solvent system to obtain the desired product (260 mg). LCMS: 449.1 [M+1]⁺

Step-4: Synthesis of 5-(5-(2-(azetidin-3-ylmethoxy)phenyl) isoxazol-3-ylamino) pyrazine-2-carbonitrile: To a solution of tert-butyl 3-((2-(3-(5-cyanopyrazin-2-ylamino) isoxazol-5-yl) phenoxy)methyl) azetidine-1-carboxylate (260 mg, 0.580 mmol, 1.0 eq) in DCM (10.0 mL), TFA (0.5 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (210 mg, trifluoroacetate salt). LCMS: 349.5 [M+1]⁺98.73 @ 220 nm; 99.64 @ 254 nm. ¹H NMR (400 MHZ, DMSO-d6, After D₂O Addition) δ 8.80 (s, 1H), 8.54 (s, 1H), 7.87 (d, J=7.7 Hz, 1H), 7.52 (t, J=8.0 Hz, 1H), 7.23 (d, J=8.5 Hz, 1H), 7.18 (d, J=12.8 Hz, 2H), 4.34 (d, J=7.3 Hz, 2H), 4.15 (t, J=9.8 Hz, 2H), 3.94 (t, J=9.3 Hz, 2H), 3.48-3.28 (m, 1H)

### Example-18: Synthesis of 5-(5-(2-(1-sec-butylazetidin-3-yloxy)phenyl) isoxazol-3-ylamino) pyrazine-2-carbonitrile (Compound 1.17)

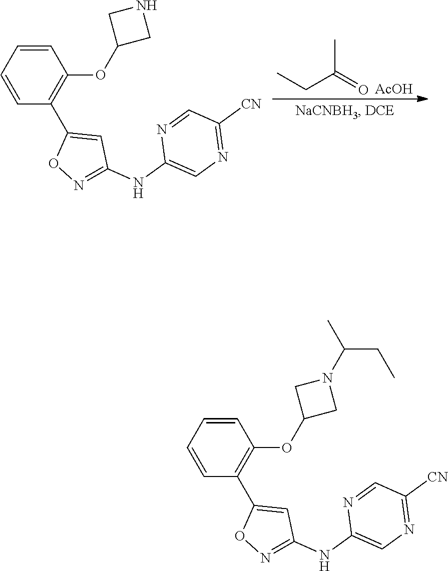

Step-1: Synthesis of 5-(5-(2-(1-sec-butylazetidin-3-yloxy)phenyl) isoxazol-3-ylamino) pyrazine-2-carbonitrile: To a stirred solution of 5-(5-(2-(azetidin-3-yloxy)phenyl) isoxazol-3-ylamino) pyrazine-2-carbonitrile (70 mg, 0.209 mmol, 1 eq) in dichloroethane was added acetic acid (0.1 mL) and butan-2-one (75 mg, 1.05 mmol, 5 eq). The resultant reaction mixture was stirred at room temperature for 1 h. Then the reaction mixture was cooled to 0° C. and sodium cyanoborohydride (66 mg, 1.05 mmol, 5 eq) was added to the reaction mixture. The reaction mixture was stirred for 3 h at RT. Product formation was confirmed by TLC and LCMS. The reaction mixture was quenched using saturated sodium bicarbonate (2×5 mL) and the organic layer as separated and concentrated in vacuum to provide the crude. The crude was purified by R-HPLC to yield desired compound (10 mg, trifluoroacetate salt). LCMS: 391.5 [M+1]⁺99.84 @ 220 nm 99.63 @ 254 nm. H NMR (400 MHZ, DMSO-d6 after D₂O addition) δ 8.74 (s, 1H), 8.63 (s, 1H), 7.90 (d, J=7.7 Hz, 1H), 7.55-7.47 (m, 1H), 7.33 (s, 1H), 7.22 (t, J=7.5 Hz, 1H), 6.92 (s, 1H), 5.26-5.14 (m, 1H), 4.64 (s, 2H), 4.22 (dd, J=12.5, 4.5 Hz, 2H), 3.28 (s, 1H), 1.66 (d, J=12.2 Hz, 1H), 1.32 (dt, J=14.5, 7.6 Hz, 1H), 1.13 (d, J=6.3 Hz, 3H), 0.87 (t, J=7.3 Hz, 3H).

### Example-19: Synthesis of 5-(5-(2-methoxy-4-(1,2,3,6-tetrahydropyridin-4-yl)phenyl) isoxazol-3-ylamino) pyrazine-2-carbonitrile (Compound 1.19)

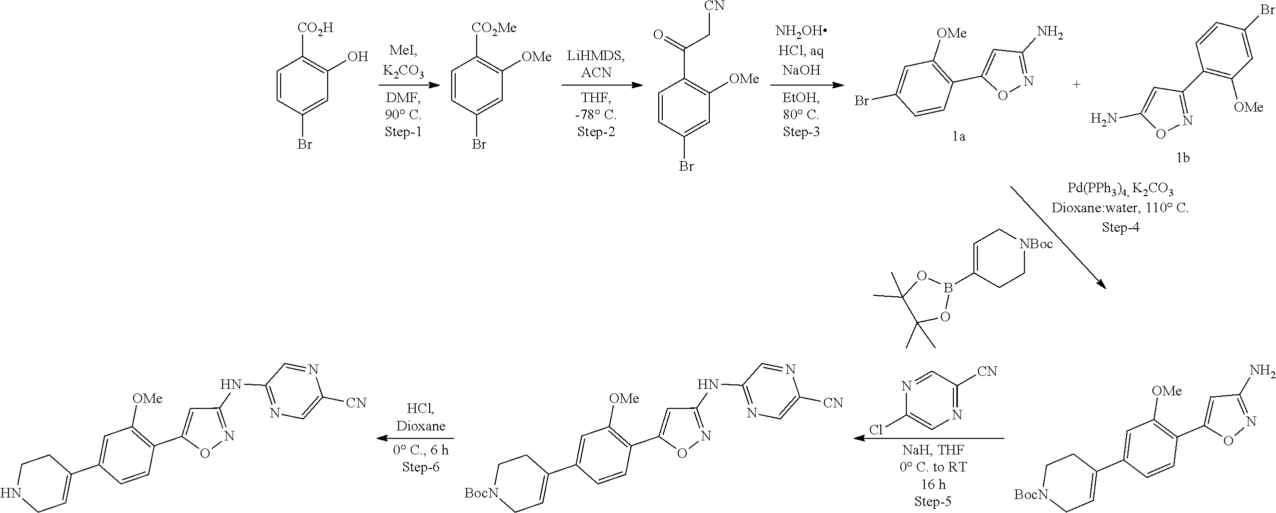

Step 1-3: Same as in shown in synthesis of Compound 1.9

Step-4: Synthesis of tert-butyl 4-(4-(3-aminoisoxazol-5-yl)-3-methoxyphenyl)-5,6-dihydropyridine-1 (2H)-carboxylate: A solution of 5-(4-bromo-2-methoxyphenyl) isoxazol-3-amine (1.2 g, 4.46 mmol, 1.0 eq) and tert-butyl 4-(4,4,5,5-tetramethyl-1,3,2-dioxaborolan-2-yl)-5,6-dihydropyridine-1 (2H)-carboxylate (1.52 g, 4.91 mmol, 1.1 eq) in Dioxane: H₂O (10 mL: 2.0 mL) was purged with N₂ for 5 minutes. K₂CO₃ (1.54 g, 11.1 mmol, 2.5 eq) was added to the reaction mixture and purged again for 5 minutes, then tetrakis (triphenylphosphine) palladium (0) (515 mg, 0.445 mmol, 0.1 eq) was added and the reaction mixture was stirred at 110° C. for 16 h. Product formation was confirmed by TLC and LCMS. The reaction mixture was quenched with ice water (50 mL) and extracted with ethyl acetate (3×30 mL). The organic layer was washed with brine (30 mL) and dried over Na₂SO₄ and then concentrated under reduced pressure to get the crude. The crude was purified by flash chromatography by using 24 g RediSep® column in ethyl acetate: hexane solvent system to obtain the desired compound (500 mg). LCMS: 372.4 [M+1]⁺

Step-5: Synthesis of tert-butyl 4-(4-(3-(5-cyanopyrazin-2-ylamino) isoxazol-5-yl)-3-methoxyphenyl)-5,6-dihydropyridine-1 (2H)-carboxylate: To a solution of tert-butyl 4-(4-(3-aminoisoxazol-5-yl)-3-methoxyphenyl)-5,6-dihydropyridine-1 (2H)-carboxylate (250 mg, 0.673 mmol, 1.0 eq) in THE (5.0 mL) at 0° C. was added sodium hydride (60% dispersion in mineral oil) (134 mg, 3.37 mmol, 5.0 eq) portion wise over the period of 10 min. To this reaction mixture was added 5-chloropyrazine-2-carbonitrile (122 mg, 0.875 mmol, 1.3 eq) was added and it was stirred at RT for 16 h. Product formation was confirmed by TLC and LCMS. After the completion of reaction, the reaction mixture was quenched with ice water (10 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired compound (120 mg). LCMS: 475.3 [M+1]⁺

Step-6: Synthesis of 5-(5-(2-methoxy-4-(1,2,3,6-tetrahydropyridin-4-yl)phenyl) isoxazol-3-ylamino) pyrazine-2-carbonitrile: To a solution of tert-butyl 4-(4-(3-(5-cyanopyrazin-2-ylamino) isoxazol-5-yl)-3-methoxyphenyl)-5,6-dihydropyridine-1 (2H)-carboxylate (120 mg, 0.253 mmol, 1 eq) in Dioxane (3.0 mL), 4.0M HCl in dioxane (3.0 mL) was added at 0° C. and kept for stirring for 6 h. The reaction mixture was concentrated under reduced pressure to get the crude. The crude was triturated with diethylether (2×5 mL) to yield the desired compound (20 mg, Hydrochloride salt). LCMS: 375.5 [M+1]⁺93.97 @220 nm; 93.79@ 254 nm 1H NMR (400 MHZ, DMSO-d6, After D₂O Addition) δ 8.77 (s, 1H), 8.63 (s, 1H), 7.86 (d, J=8.6 Hz, 1H), 7.23 (t, J=6.3 Hz, 3H), 6.38 (s, 1H), 3.99 (s, 3H), 3.78 (s, 2H), 3.33 (t, J=6.0 Hz, 2H), 2.73 (s, 2H).

### Example-20: Synthesis of 5-(5-(2-(3-aminocyclobutoxy)phenyl) isoxazol-3-ylamino) pyrazine-2-carbonitrile (Compound 1.20)

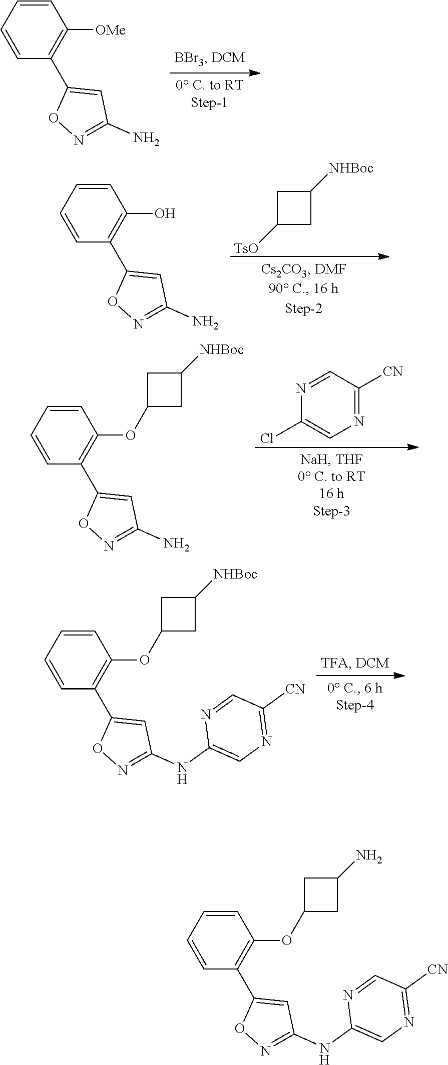

Step-1: Synthesis of 2-(3-aminoisoxazol-5-yl) phenol: Same as described in Compound 1.1

Step-2: Synthesis of tert-butyl 3-(2-(3-aminoisoxazol-5-yl) phenoxy)cyclobutylcarbamate: To a solution of 2-(3-aminoisoxazol-5-yl) phenol (300 mg, 1.7 mmol, 1.0 eq) in DMF (5 mL) was added 3-(tert-butoxycarbonylamino)cyclobutyl 4-methylbenzenesulfonate (synthesized as described in WO2018234978 A1) (698 mg, 2.04 mmol, 1.2 eq) and Cs₂CO₃ (1.11 g, 3.41 mmol, 2.0 eq) in it. The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product as a colorless liquid (200 mg). LCMS: 346.3 [M+1]⁺

Step-3: Synthesis tert-butyl 3-(2-(3-(5-cyanopyrazin-2-ylamino) isoxazol-5-yl) phenoxy)cyclobutylcarbamate: To a solution of tert-butyl (3-(2-(3-aminoisoxazol-5-yl) phenoxy)cyclobutyl) carbamate (200 mg, 0.579 mmol, 1.0 eq) in THE (10.0 mL) at 0° C. was added sodium hydride (60% dispersion in mineral oil) (115 mg, 2.9 mmol, 5.0 eq) portion wise over the period of 10 min. The reaction mixture was stirred for 30 min at 0° C. followed by addition of 5-chloropyrazine-2-carbonitrile (97 mg, 0.695 mmol, 1.2 eq). The reaction mixture was allowed to stir at RT for 16 h. Product formation was confirmed by TLC and LCMS. After the completion of reaction, the reaction mixture was quenched with ice water (30 mL) and extracted with ethyl acetate (3×50 mL). Collective organic layer was washed with brine (20 mL) and dried over anhydrous sodium sulfate and then concentrated under reduced pressure to get the crude which was used as such in the next step (100 mg). LCMS: 449.3 [M+1]⁺

Step-4: Synthesis of 5-(5-(2-(3-aminocyclobutoxy)phenyl) isoxazol-3-ylamino) pyrazine-2-carbonitrile: To a solution of tert-butyl 3-(2-(3-(5-cyanopyrazin-2-ylamino) isoxazol-5-yl) phenoxy)cyclobutylcarbamate (100 mg, 0.223 mmol, 1.0 eq) in DCM (4.0 mL), TFA (0.1 mL) was added at 0° C. and kept for stirring for 6 h. Product formation was confirmed by TLC and LCMS. Then the reaction mixture was concentrated under reduced pressure to get the crude. The crude was purified by R-HPLC to yield desired compound (10 mg, trifluoroacetate salt). LCMS: 349.5 [M+1]⁺97.3 @ 220 nm 98.04 @ 254 nm. ¹H NMR (400 MHZ, DMSO-d6, After D₂O Addition) δ 8.76 (d, J=8.1 Hz, 1H), 8.62 (d, J=5.3 Hz, 1H), 7.89 (d, J=7.4 Hz, 1H), 7.50 (t, J=7.8 Hz, 1H), 7.43 (d, J=6.1 Hz, 1H), 7.19-7.11 (m, 1H), 7.00 (dd, J=29.2, 8.3 Hz, 1H), 5.12 (s, 1H), 3.91 (dd, J=14.2, 7.1 Hz, 1H), 2.98 (s, 1H), 2.75-2.65 (m, 2H), 2.58-2.55 (m, 1H).

### Example-21: Synthesis of 5-(5-(2-(2-azaspiro[3.3]heptan-6-yloxy)phenyl) isoxazol-3-ylamino) pyrazine-2-carbonitrile (Compound 1.21)

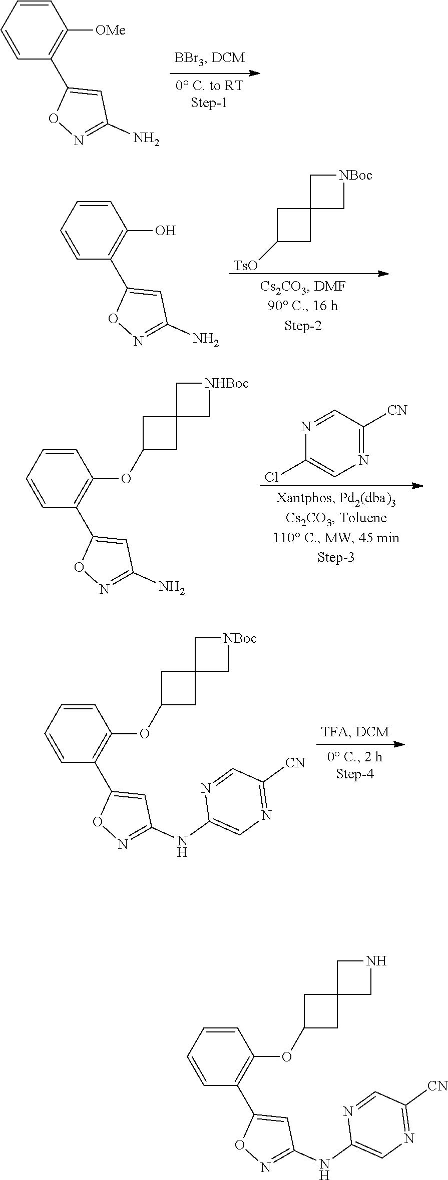

Step-1: Synthesis of 2-(3-aminoisoxazol-5-yl) phenol: Same as described in Compound 1.1

Step-2: Synthesis of tert-butyl 6-(2-(3-aminoisoxazol-5-yl) phenoxy)-2-azaspiro[3.3]heptane-2-carboxylate: To a solution of 2-(3-aminoisoxazol-5-yl) phenol (240 mg, 1.36 mmol, 1.0 eq) in DMF (5 mL) was added tert-butyl 6-(tosyloxy)-2-azaspiro[3.3]heptane-2-carboxylate (synthesized as described in WO 2021124222 A1) (500 mg, 1.36 mmol, 1.0 eq) and Cs₂CO₃ (577 mg, 1.77 mmol, 1.3 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 12 g RediSep® column in ethyl acetate: hexane solvent system to obtain the desired product (210 mg). LCMS: 372.3 [M+1]⁺

Step-3: Synthesis tert-butyl 6-(2-(3-(5-cyanopyrazin-2-ylamino) isoxazol-5-yl) phenoxy)-2-azaspiro[3.3]heptane-2-carboxylate: A solution of tert-butyl 6-(2-(3-aminoisoxazol-5-yl) phenoxy)-2-azaspiro[3.3]heptane-2-carboxylate (200 mg, 0.538 mmol, 1.0 eq) and 5-chloropyrazine-2-carbonitrile (90 mg, 0.646 mmol, 1.2 eq) in toluene (5 mL) was purged with N₂ for 5 minutes followed by addition of Cs₂CO₃ (525 mg, 1.61 mmol, 3.0 eq). The resultant reaction mixture was again purged for 5 minutes, then Xantphos (18 mg, 0.032 mmol, 0.06 eq) and Pd₂dba₃ (39 mg, 0.043 mmol, 0.08 eq) was added and the reaction mixture was subjected to microwave for 45 minutes at 110° C. Product formation was confirmed by TLC and LCMS. After the completion of reaction, the reaction mixture was quenched with ice water (20 mL) and extracted with ethyl acetate (3×15 mL). Collective organic layer was washed with brine (20 mL) and dried over anhydrous sodium sulfate and then concentrated under reduced pressure to get the crude. The crude was purified by combi flash chromatography by using 12 g RediSep® column and by eluting in ethyl acetate: hexane solvent system to obtain the desired compound (70 mg). LCMS: 475.3 [M+1]⁺

Step-4: Synthesis of 5-(5-(2-(2-azaspiro[3.3]heptan-6-yloxy)phenyl) isoxazol-3-ylamino) pyrazine-2-carbonitrile: To a solution of tert-butyl 6-(2-(3-(5-cyanopyrazin-2-ylamino) isoxazol-5-yl) phenoxy)-2-azaspiro[3.3]heptane-2-carboxylate (70 mg, 0.147 mmol, 1.0 eq) in DCM (4.0 mL) TFA (0.4 mL) was added at 0° C. and kept for stirring for 2 h. Product formation was confirmed by TLC and LCMS. Then the reaction mixture was concentrated under reduced pressure to get the crude. The crude was purified by R-HPLC to yield desired compound (10 mg, trifluoroacetate salt). LCMS: 375.5 [M+1]⁺99.66 @ 220 nm; 99.72 @ 254 nm. 1H NMR (400 MHZ, DMSO-d6, after D₂O addition) δ 8.79 (s, 1H), 8.60 (s, 1H), 7.87 (d, J=7.8 Hz, 1H), 7.47 (t, J=7.8 Hz, 1H), 7.36 (s, 1H), 7.13 (t, J=7.6 Hz, 1H), 6.99 (d, J=8.3 Hz, 1H), 4.81 (q, J=6.6 Hz, 1H), 4.03 (d, J=18.9 Hz, 4H), 2.89 (dd, J=12.7, 6.4 Hz, 2H), 2.44-2.31 (m, 2H).

### Example-22: Synthesis of 5-(3-(2-methoxy-4-(1-methyl-1,2,3,6-tetrahydropyridin-4-yl)phenyl) isoxazol-5-ylamino) pyrazine-2-carbonitrile (Compound 1.22)

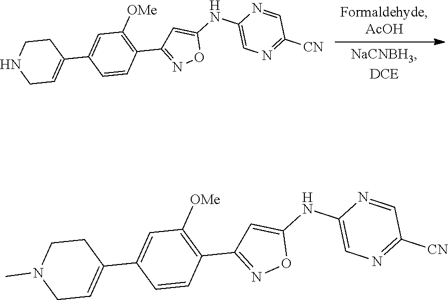 [SMILES: COc1cc(C2=CCNCC2)ccc1-c1cc(Nc2cnc(C#N)cn2)on1 ; COc1cc(C2=CCN(C)CC2)ccc1-c1cc(Nc2cnc(C#N)cn2)on1]

Step-1: Synthesis of 5-(3-(2-methoxy-4-(1-methyl-1,2,3,6-tetrahydropyridin-4-yl)phenyl) isoxazol-5-ylamino) pyrazine-2-carbonitrile: To a stirred solution of 5-(3-(2-methoxy-4-(1,2,3,6-tetrahydropyridin-4-yl)phenyl) isoxazol-5-ylamino) pyrazine-2-carbonitrile (80 mg, 0.213 mmol, 1 eq) in dichloroethane (4 mL) was added acetic acid (0.1 mL) and formaldehyde (37% solution) (0.032 mL, 1.07 mmol, 5 eq). The resultant reaction mixture was stirred at room temperature for 1 h. Then the reaction mixture was cooled to 0° C. and sodium cyanoborohydride (33 mg, 0.534 mmol, 2.5 eq) was added to the reaction mixture. The reaction mixture was stirred for 5 h at RT. Product formation was confirmed by TLC and LCMS. The reaction mixture was quenched using saturated sodium bicarbonate (2×5 mL) and the organic layer as separated and concentrated in vacuum to provide the crude. The crude was purified by R-HPLC to yield desired compound (4 mg, formate salt). LCMS: 389.5 [M+1]⁺97.23@ 220 nm; ¹H NMR (400 MHZ, DMSO-d6) δ 8.77 (s, 1H), 8.34 (s, 1H), 8.23 (s, 1H, formate CH), 7.70 (d, J=8.1 Hz, 1H), 7.21-7.10 (m, 2H), 6.81 (s, 1H), 6.31 (s, 1H), 3.89 (s, 3H), 3.41 (s, 2H), 2.96 (s, 2H), 2.64 (s, 2H), 2.54 (s, 3H).

### Example-23: Synthesis of 5-(5-(2-(pyrrolidin-3-ylmethoxy)phenyl) isoxazol-3-ylamino) pyrazine-2-carbonitrile (Compound 1.23)

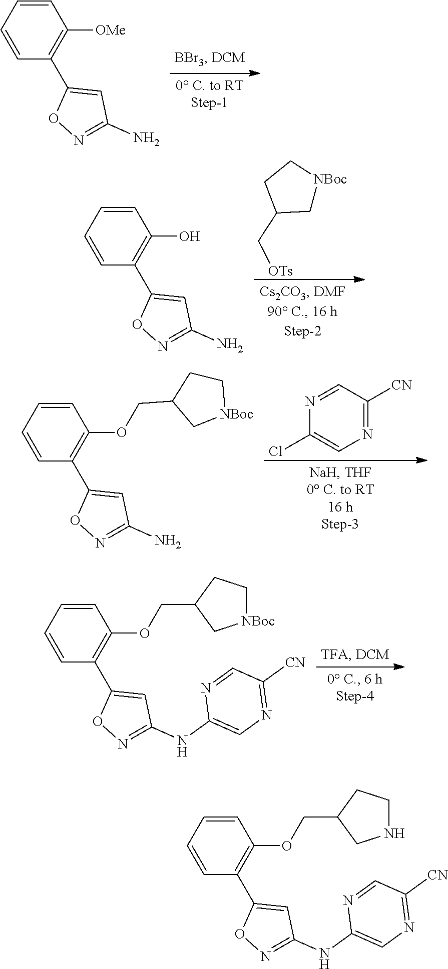

Step-1: Synthesis of 2-(3-aminoisoxazol-5-yl) phenol: Same as described in Compound 1.1

Step-2: Synthesis of tert-butyl 3-((2-(3-aminoisoxazol-5-yl) phenoxy)methyl) pyrrolidine-1-carboxylate: To a solution of 2-(3-aminoisoxazol-5-yl) phenol (300 mg, 1.70 mmol, 1.0 eq) in DMF was added tert-butyl 3-(tosyloxymethyl) pyrrolidine-1-carboxylate (synthesized as described in J. Med. Chem, 2020, 63 (19), 11054-84) (726.1 mg, 2.04 mmol, 1.2 eq) and Cs₂CO₃ (1.1 g, 3.40 mmol, 2.0 eq). The resultant reaction mixture was stirred at 90° C. for 16 h. Product formation was confirmed by TLC and LCMS. After the completion of reaction, the reaction mixture was quenched with ice water (30 mL) and extracted with ethyl acetate (2×20 mL). Collective organic layer was washed with brine (20 mL) and dried over anhydrous sodium sulfate and then concentrated under reduced pressure to get the crude. The crude was purified by flash chromatography by using 4 g RediSep® column and by using ethyl acetate: hexane solvent system to obtain the desired compound (300 mg) as a colorless liquid. LCMS: 360.3 [M+1]⁺

Step-3: Synthesis of tert-butyl 3-((2-(3-(5-cyanopyrazin-2-ylamino) isoxazol-5-yl) phenoxy)methyl) pyrrolidine-1-carboxylate: To a solution of tert-butyl 3-((2-(3-aminoisoxazol-5-yl) phenoxy)methyl) pyrrolidine-1-carboxylate (300 mg, 0.835 mmol, 1.0 eq) in THF (5 ML) at 0° C. was added sodium hydride (60% dispersion in mineral oil) (167 mg, 4.17 mmol, 5.0 eq) portionwise over the period of 10 min. The reaction mixture was then stirred for 30 min at 0° C. followed by addition of 5-chloropyrazine-2-carbonitrile (232.1 mg, 1.67 mmol, 2.0 eq) and it was stirred at RT for 16 h. Product formation was confirmed by TLC and LCMS. The reaction mixture was quenched using ice-cold water (30 mL) and extracted using ethyl acetate (2×50 mL). The combined organic layer was washed with brine (30 mL), dried over anhydrous sodium sulfate and concentrated under reduced pressure to get the crude (350 mg) as a pale yellow solid which was used as such in the next step. LCMS: 463.2 [M+1]⁺

Step-4: Synthesis of 5-(5-(2-(pyrrolidin-3-ylmethoxy)phenyl) isoxazol-3-ylamino) pyrazine-2-carbonitrile: To a solution of tert-butyl 3-((2-(3-(5-cyanopyrazin-2-ylamino) isoxazol-5-yl) phenoxy)methyl) pyrrolidine-1-carboxylate (200 mg, 0.431 mmol, 1.0 eq) in DCM (5 mL), TFA (0.5 mL) was added at 0° C. and kept for stirring for 6 h. The reaction mixture was concentrated under reduced pressure to get the crude. The crude was purified by R-HPLC to yield desired compound (50 mg, trifluoroacetate salt). LCMS: 363.5 [M+1]⁺97.53 @ 220 nm 97.76 @ 254 nm. ¹H NMR (400 MHZ, Methanol-d4) δ 8.64 (s, 1H), 8.59 (s, 1H), 7.89 (dd, J=7.9, 1.8 Hz, 1H), 7.53-7.46 (m, 1H), 7.30 (s, 1H), 7.21 (d, J=8.5 Hz, 1H), 7.14 (t, J=7.6 Hz, 1H), 4.28 (dd, J=9.4, 6.2 Hz, 1H), 4.22-4.14 (m, 1H), 3.63 (dd, J=11.9, 7.9 Hz, 1H), 3.50-3.41 (m, 1H), 3.41-3.36 (m, 1H), 3.29-3.23 (m, 1H), 3.06 (q, J=7.5 Hz, 1H), 2.48-2.34 (m, 1H), 2.06-1.93 (m, 1H).

### Example-24: Synthesis of 5-(5-(3-(piperidin-4-ylmethoxy) naphthalen-2-yl)-1H-pyrazol-3-ylamino) pyrazine-2-carbonitrile (Compound 2.1)

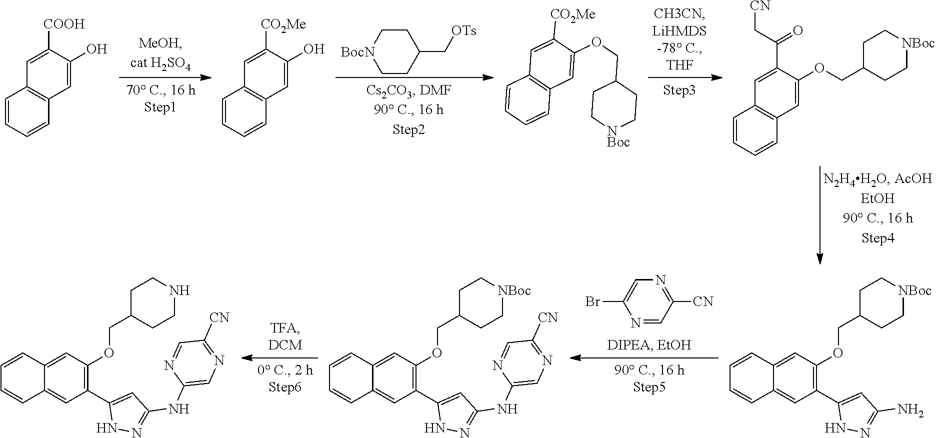

Step-1: Synthesis of methyl 3-hydroxy-2-naphthoate: Synthesized as described in WO2000009169A1.

Step-2: Synthesis of tert-butyl 4-((3-(methoxycarbonyl) naphthalen-2-yloxy)methyl) piperidine-1-carboxylate: To a solution of tert-butyl 4-(tosyloxymethyl) piperidine-1-carboxylate (2.0 g, 5.41 mmol, 1.0 eq) in DMF (40 mL), Cs₂CO₃ (4.41 g, 13.53 mmol, 2.5 eq) was added followed by addition of methyl 3-hydroxy-2-naphthoate (1.42 g, 7.04 mmol, 1.3 eq) and the reaction mixture was kept for stirring at 90° C. overnight. Reaction progress was monitored by TLC and LCMS. The reaction mixture was quenched by addition of ice water (50 mL) and extracted with ethyl acetate (3×25 mL). Collective organic layer was washed with brine (25 mL), dried over Na₂SO₄ and then concentrated under reduced pressure to get the crude. The crude was purified by flash chromatography by using 24 g RediSep® column and by eluting in 30% ethyl acetate: hexane solvent system to obtain the desired compound (1.4 g. LCMS: 300.3 [M+H−100]⁺; de-Boc peak

Step-3: Synthesis of tert-butyl 4-((3-(2-cyanoacetyl) naphthalen-2-yloxy)methyl) piperidine-1-carboxylate: To a solution of acetonitrile (10 mL) in THF (8 mL) was added LiHMDS (1M in THF) (10.5 mL, 10.51 mmol, 3.0 eq) at −78° C. and stirred for 2 h at same temperature. Then tert-butyl 4-((3-(methoxycarbonyl) naphthalen-2-yloxy)methyl) piperidine-1-carboxylate (1.4 g, 3.5 mmol, 1.0 eq) was added and stirred for 2 h. Product formation was confirmed by TLC and LCMS. The reaction mixture was quenched with water (20 mL) and extracted with ethyl acetate (3×15 mL). Collective organic layer was washed with brine (20 mL) and dried over Na₂SO₄ and then concentrated under reduced pressure to get the crude. The crude by purified by flash chromatography by using 12 g RediSep® column and by eluting in 30% ethyl acetate: hexane solvent system to obtain the desired compound (800 mg). LCMS: 353.3 [M+H−56]⁺

Step-4: Synthesis of tert-butyl 4-((3-(3-amino-1H-pyrazol-5-yl) naphthalen-2-yloxy)methyl) piperidine-1-carboxylate: To a solution of tert-butyl 4-((3-(2-cyanoacetyl) naphthalen-2-yloxy)methyl) piperidine-1-carboxylate (500 mg, 1.22 mmol, 1.0 eq) in EtOH (5.0 mL) was added hydrazine hydrate (0.3 mL, 6.12 mmol, 5.0 eq) followed by dropwise addition of acetic acid (5 drops) and the resultant reaction mixture was kept for stirring at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (300 mg). LCMS: 423.3 [M+H]⁺

Step-5: Synthesis of tert-butyl 4-((3-(3-(5-cyanopyrazin-2-ylamino)-1H-pyrazol-5-yl) naphthalen-2-yloxy)methyl) piperidine-1-carboxylate: To a solution of tert-butyl 4-((3-(3-amino-1H-pyrazol-5-yl) naphthalen-2-yloxy)methyl) piperidine-1-carboxylate (300 mg, 0.710 mmol, 1.0 eq) in EtOH (5.0 mL) was added DIPEA (0.3 mL, 1.78 mmol, 2.5 eq) followed by addition of 5-bromopyrazine-2-carbonitrile (261 mg, 1.42 mmol, 2.0 eq) and the reaction mixture was stirred at 90° C. for overnight. Product formation was confirmed by TLC and LCMS. The reaction mixture was concentrated under reduced pressure to get the crude. The crude by purified by flash chromatography by using 12 g RediSep® column and by eluting in 2% MeOH/DCM solvent system to obtain the desired compound (150 mg). LCMS: 426.3 [M+H−Boc]⁺

Step-7: Synthesis of 5-(5-(3-(piperidin-4-ylmethoxy) naphthalen-2-yl)-1H-pyrazol-3-ylamino) pyrazine-2-carbonitrile: To a solution of tert-butyl 4-((3-(3-(5-cyanopyrazin-2-ylamino)-1H-pyrazol-5-yl) naphthalen-2-yloxy)methyl) piperidine-1-carboxylate (150 mg, 0.285 mmol, 1.0 eq) in DCM (5.0 mL), TFA (113 mg, 0.998 mmol, 3.5 eq) was added at 0° C. and kept for stirring for 2 h. The reaction mixture was concentrated under reduced pressure to get the crude. The crude was purified by R-HPLC to yield desired compound (15 mg, formate salt). LCMS: 426.3 [M+1]+; 99.85 @ 220 nm, 99.88 @ 254 nm. ¹H NMR (400 MHZ, DMSO-d6) δ 8.63 (s, 1H), 8.50 (s, 1H), 8.38 (s, 1H), 8.21 (s, 1H), 7.85 (dd, J=15.1, 8.2 Hz, 2H), 7.48 (d, J=8.6 Hz, 2H), 7.40 (t, J=7.5 Hz, 1H), 7.13 (s, 1H), 4.09 (d, J=6.8 Hz, 2H), 3.29 (d, J=12.1 Hz, 2H), 2.89 (t, J=12.3 Hz, 2H), 2.23 (s, 1H), 2.03 (d, J=13.1 Hz, 2H), 1.49 (d, J=14.1 Hz, 2H).

### Example-25: Synthesis of 5-(5-(3-(azetidine-3-ylmethoxy) naphthalen-2-yl)-1H-pyrazol-3-ylamino) pyrazine-2-carbonitrile (Compound 2.2)

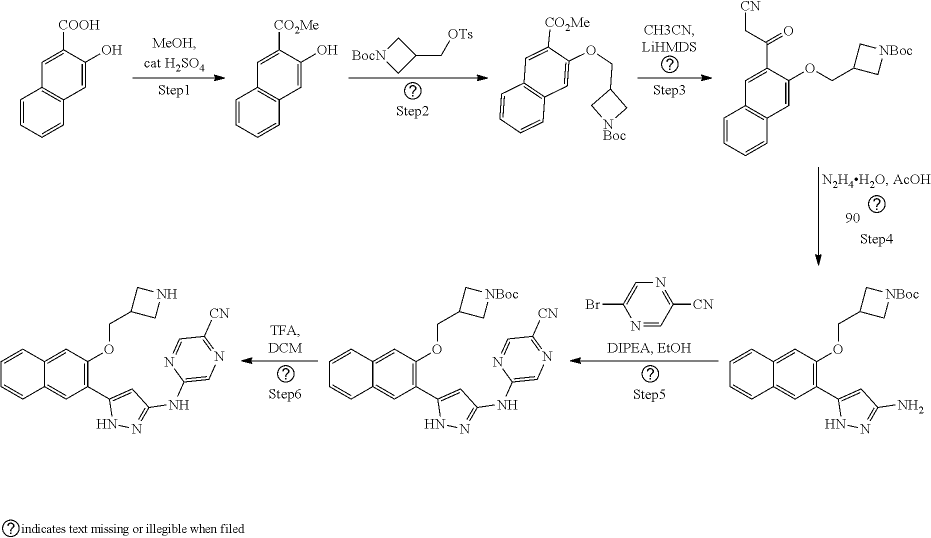

Step-1: Synthesis of methyl 3-hydroxy-2-naphthoate: Synthesized as described in WO2000009169A1

Step-2: Synthesis of tert-butyl 3-((3-(methoxycarbonyl) naphthalen-2-yloxy)methyl) azetidine-1-carboxylate: To a solution of tert-butyl 3-(tosyloxymethyl) azetidine-1-carboxylate (2.0 g, 5.86 mmol, 1.0 eq) in DMF (40 mL), Cs₂CO₃ (4.77 g, 14.64 mmol, 2.5 eq) was added followed by addition of methyl 3-hydroxy-2-naphthoate (1.54 g, 7.62 mmol, 1.3 eq) and the reaction mixture was stirred at 90° C. overnight. Reaction progress was monitored by TLC and LCMS. The reaction mixture was quenched with ice water (50 mL) and extracted with ethyl acetate (3×25 mL). Collective organic layer was washed with brine (25 mL) and dried over Na₂SO₄ and then concentrated under reduced pressure to get the crude. The crude by purified by flash chromatography by using 24 g RediSep® column and by eluting in 20% ethyl acetate: hexane solvent system to obtain the desired compound (1.8 g). LCMS: 372.4 [M+H]⁺

Step-3: Synthesis of tert-butyl 3-((3-(2-cyanoacetyl) naphthalen-2-yloxy)methyl) azetidine-1-carboxylate: To a solution of ACN (10 mL) in THF (8.0 mL) was added LiHMDS (1M in THF) (14.54 mL, 14.54 mmol, 3.0 eq) at −78° C. and stirred for 2 h at same temperature. Then tert-butyl 3-((3-(methoxycarbonyl) naphthalen-2-yloxy)methyl) azetidine-1-carboxylate (1.8 g, 4.85 mmol, 1.0 eq) was added and stirred for 2 h. Product formation was confirmed by TLC and LCMS. The reaction mixture was quenched with water (20 mL) and extracted with ethyl acetate (3×15 mL). Collective organic layer was washed with brine (20 mL) and dried over Na₂SO₄ and then concentrated under reduced pressure to get the crude. The crude was purified by flash chromatography by using 12 g RediSep® column and by eluting in 30% E.A/Hexane solvent system obtain the desired compound (1 g). LCMS: 381.3 [M+H]⁺

Step-4: Synthesis of tert-butyl 3-((3-(3-amino-1H-pyrazol-5-yl) naphthalen-2-yloxy)methyl) azetidine-1-carboxylate: To a solution of tert-butyl 3-((3-(2-cyanoacetyl) naphthalen-2-yloxy)methyl) azetidine-1-carboxylate (500 mg, 1.31 mmol, 1.0 eq) in EtOH (5.0 mL) was added hydrazine hydrate (0.3 mL, 6.57 mmol, 5.0 eq) followed by dropwise addition of acetic acid (5 drops) and the reaction mixture was stirred at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (300 mg). LCMS: 395.4 [M+H]⁺

Step-5: Synthesis of tert-butyl 3-((3-(3-(5-cyanopyrazin-2-ylamino)-1H-pyrazol-5-yl) naphthalen-2-yloxy)methyl) azetidine-1-carboxylate: To a solution of tert-butyl 3-((3-(3-amino-1H-pyrazol-5-yl) naphthalen-2-yloxy)methyl) azetidine-1-carboxylate (150 mg, 0.380 mmol, 1.0 eq) in EtOH (5 mL) was added DIPEA (0.14 mL, 0.76 mmol, 2 eq) followed by addition of 5-bromopyrazine-2-carbonitrile (140 mg, 0.76 mmol, 2.0 eq) and the reaction mixture was stirred overnight at 90° C. Product formation was confirmed by TLC and LCMS. The reaction mixture was concentrated under reduced pressure to get the crude. The crude was purified was purified by flash chromatography by using 12 g RediSep® column and by using 2% MeOH/DCM solvent system to get the desired compound (100 mg).LCMS: 442.3 [(M+1)−56]⁺

Step-6: Synthesis of 5-(5-(3-(azetidine-3-ylmethoxy) naphthalen-2-yl)-1H-pyrazol-3-ylamino) pyrazine-2-carbonitrile: To a solution of tert-butyl 3-((3-(3-((5-cyanopyrazin-2-ylamino)-1H-pyrazol-5-yl) naphthalen-2-yloxy)methyl) azetidine-1-carboxylate (100 mg, 0.201 mmol, 1.0 eq) in DCM (5.0 mL) TFA (80 mg, 0.704 mmol, 3.5equiv) was added at 0° C. and kept for stirring for 2 h. Then the reaction mixture was concentrated under reduced pressure to get the crude. The crude was purified by R-HPLC to yield desired compound (15 mg, formate salt). LCMS: 398.3 [M+H]⁺99.89 @ 220 nm, 99.85 @ 254 nm ¹H NMR (400 MHZ, DMSO-d6) δ 8.71 (s, 1H), 8.51 (s, 1H), 8.35 (s, 1H), 8.31 (s, 1H), 7.92 (d, J=8.3 Hz, 1H), 7.84 (d, J=8.2 Hz, 1H), 7.52 (d, J=3.1 Hz, 1H), 7.49 (d, J=7.8 Hz, 1H), 7.40 (t, J=7.4 Hz, 1H), 7.17 (s, 1H), 4.40 (d, J=4.7 Hz, 2H), 4.05 (t, J=8.7 Hz, 2H), 3.60 (s, 2H), 3.21-3.16 (m, 1H).

### Example-26: Synthesis of 5-(5-(3-(morpholin-2-ylmethoxy) naphthalen-2-yl)-1H-pyrazol-3-ylamino) pyrazine-2-carbonitrile (Compound 2.3)

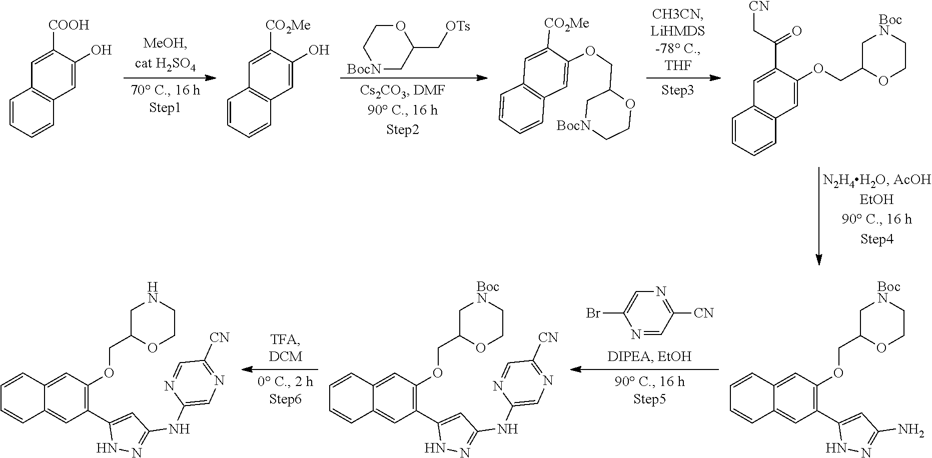

Step-1: Synthesis of methyl 3-hydroxy-2-naphthoate: Synthesized as described in WO2000009169A1

Step-2: Synthesis of tert-butyl 2-((3-(methoxycarbonyl) naphthalen-2-yloxy)methyl) morpholine-4-carboxylate: To a solution of tert-butyl 2-(tosyloxy)methyl) morpholine-4-carboxylate (1.5 g, 4.04 mmol, 1.0 eq) in DMF (40 mL) Cs₂CO₃ (3.2 g, 10.10 mmol, 2.5 eq) was added followed by addition of methyl 3-hydroxy-2-naphthoate (1.06 g, 5.25 mmol, 1.3 eq) and the reaction mixture was stirred at 90° C. overnight. Reaction progress was monitored by TLC and LCMS. The reaction mixture was quenched with ice water (50 mL) and extracted with ethyl acetate (3×25 mL). Collective organic layer was washed with brine (25 mL) and dried over Na₂SO₄ and then concentrated under reduced pressure to get the crude. The crude was purified by flash chromatography by using 20% E.A/Hexane solvent system to yield the desired compound (1.3 g).LCMS: 302.4 [M+H−100]

Step-3: Synthesis of tert-butyl 2-((3-(2-cyanoacetyl) naphthalen-2-yloxy)methyl) morpholine-4-carboxylate: To a solution of ACN (10 mL) in THF (8.0 mL) was added LiHMDS (1M in THF) (8.97 mL, 8.97 mmol, 3.0 eq) at −78° C. and stirred for 2 h at same temperature. Then tert-butyl 2-((3-(methoxycarbonyl) naphthalen-2-yloxy)methyl) morpholine-4-carboxylate (1.2 g, 2.99 mmol, 1.0 eq) was added and stirred for 2 h.Product formation was confirmed by TLC and LCMS. The reaction mixture was quenched with water (20 mL) and extracted with ethyl acetate (3×15 mL). Collective organic layer was washed with brine (20 mL) and dried over Na₂SO₄ and then concentrated under reduced pressure to get the crude. The crude was purified by flash chromatography by using 30% ethyl acetate/Hexane solvent system to to yield the desired compound (800 mg). LCMS: 311.3 [M+H−100]⁺

Step-4: Synthesis of tert-butyl 2-((3-(3-amino-1H-pyrazol-5-yl) naphthalen-2-yloxy)methyl) morpholine-4-carboxylate: To a solution of tert-butyl 2-((3-(2-cyanoacetyl) naphthalen-2-yloxy)methyl) morpholine-4-carboxylate (500 mg, 1.22 mmol, 1.0 eq) in EtOH (5.0 mL) was added hydrazine hydrate (0.3 mL, 6.09 mmol, 5.0 eq) followed by addition of acetic acid (5 drops) and the reaction mixture was stirred at 90° C. overnight. Product formation was confirmed by TLC and LCMS. The reaction mixture was concentrated under reduced pressure to get the crude. The crude was purified by flash chromatography by using 2% MeOH/DCM solvent system to yield the desired compound (400 mg). LCMS: 325.7 [M+H−100]⁺

Step-5: Synthesis of tert-butyl 2-((3-(3-(5-cyanopyrazin-2-ylamino)-1H-pyrazol-5-yl) naphthalen-2-yloxy)methyl) morpholine-4-carboxylate: To a solution of tert-butyl 2-((3-(3-amino-1H-pyrazol-5-yl) naphthalen-2-yloxy)methyl) morpholine-4-carboxylate (200 mg, 0.471 mmol, 1.0 eq) in EtOH (5.0 mL) was added DIPEA (0.21 mL, 1.18 mmol, 2.5 eq) followed by addition of 5-bromopyrazine-2-carbonitrile (172 mg, 0.942 mmol, 2.0 eq) and the reaction mixture was stirred at 90° C. for overnight. Product formation was confirmed by TLC and LCMS. The reaction mixture was concentrated under reduced pressure to get the crude. Then the crude was purified by flash chromatography by using 2% MeOH/DCM solvent system to yield the desired compound (150 mg).LCMS: 528.3 [(M+1)]⁺

Step-6: Synthesis of 5-((5-(3-(morpholin-2-ylmethoxy) naphthalen-2-yl)-1H-pyrazol-3-yl)amino) pyrazine-2-carbonitrile: To a solution of tert-butyl 2-((3-(3-((5-cyanopyrazin-2-yl)amino)-1H-pyrazol-5-yl) naphthalen-2-yloxy)methyl) morpholine-4-carboxylate (100 mg, 0.190 mmol, 1.0 eq) in DCM (5.0 mL), TFA (75 mg, 0.663 mmol, 3.5 eq) was added at 0° C. and kept for stirring for 2 h. Then the reaction mixture was concentrated under reduced pressure to get the crude. The crude was purified by R-HPLC to yield desired compound (15 mg, formate salt). LCMS: 428.3 [M+H]⁺99.25 @ 220 nm, 99.23 @ 254 nm. ¹H NMR (400 MHZ, DMSO-d6) δ 8.68 (s, 1H), 8.46 (s, 1H), 8.28 (s, 1H), 8.23 (s, 1H), 7.88 (d, J=8.1 Hz, 1H), 7.82 (d, J=8.2 Hz, 1H), 7.52-7.46 (m, 2H), 7.40 (t, J=7.5 Hz, 1H), 7.18 (s, 1H), 4.23 (qd, J=10.4, 4.5 Hz, 2H), 4.04 (s, 1H), 3.94 (d, J=12.0 Hz, 1H), 3.64 (d, J=11.5 Hz, 1H), 3.15 (d, J=12.2 Hz, 1H), 2.93 (d, J=12.6 Hz, 1H), 2.81 (q, J=13.3, 12.8 Hz, 2H).

### Example-27: Synthesis of 5-(5-(3-(pyrrolidin-3-ylmethoxy) naphthalen-2-yl)-1H-pyrazol-3-ylamino) pyrazine-2-carbonitrile (Compound 2.4)

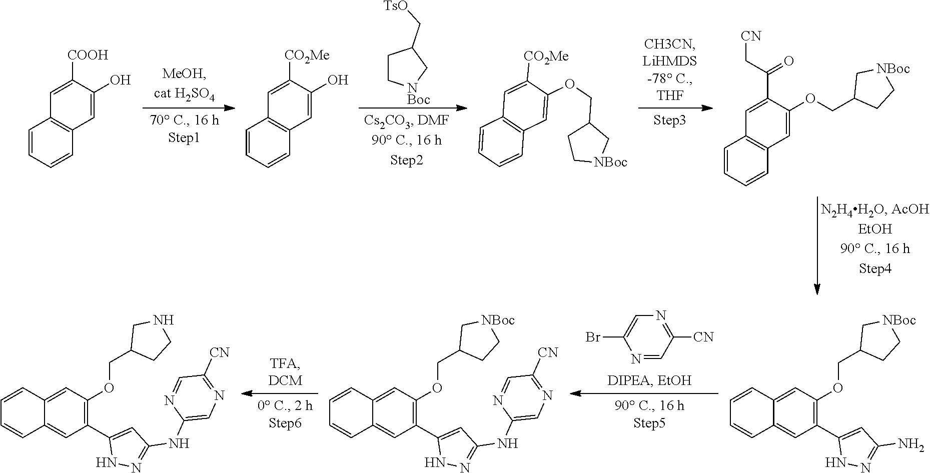

Step-1: Synthesis of methyl 3-hydroxy-2-naphthoate: Synthesized as described in WO2000009169 A1

Step-2: Synthesis of tert-butyl 3-((3-(methoxycarbonyl) naphthalen-2-yloxy)methyl) pyrrolidine-1-carboxylate: To a solution of methyl 3-hydroxy-2-naphthoate (1.37 g, 6.75 mmol, 1.2 eq) in DMF (30 mL), Cs₂CO₃ (2.75 g, 8.44 mmol, 1.5 eq) was added followed by addition of tert-butyl 3-(tosyloxymethyl) pyrrolidine-1-carboxylate (2.0 g, 5.63 mmol, 1.0 eq) and the reaction mixture was kept for stirring at 90° C. overnight. Reaction progress was monitored by TLC and LCMS. The reaction mixture was quenched by addition of ice water (50 mL) and extracted with ethyl acetate (3×25 mL). Collective organic layer was washed with brine (25 mL), dried over Na₂SO₄ and then concentrated under reduced pressure to get the crude. The crude was purified by flash chromatography by using 24 g RediSep® column and by eluting in 30% ethyl acetate: hexane solvent system to obtain the desired compound (1.62 g). LCMS: 286.3 [M+H−100]⁺; de-Boc peak

Step-3: Synthesis of tert-butyl 3-((3-(2-cyanoacetyl) naphthalen-2-yloxy)methyl) pyrrolidine-1-carboxylate: To a solution of acetonitrile (12 mL) in THF (20 mL) was added LiHMDS (1M in THF) (14.7 mL, 14.7 mmol, 3.5 eq) at −78° C. and stirred for 2 h at same temperature. Then tert-butyl 3-((3-(methoxycarbonyl) naphthalen-2-yloxy)methyl) pyrrolidine-1-carboxylate (1.62 g, 4.20 mmol, 1.0 eq) was added and stirred for 2 h. Product formation was confirmed by TLC and LCMS. The reaction mixture was quenched with water (20 mL) and extracted with ethyl acetate (3×15 mL). Collective organic layer was washed with brine (20 mL) and dried over Na₂SO₄ and then concentrated under reduced pressure to get the crude. The crude by purified by flash chromatography by using 12 g RediSep® column and by eluting in 30% ethyl acetate: hexane solvent system to obtain the desired compound (620 mg).LCMS: 295.3 [M+H−100]⁺

Step-4: Synthesis of tert-butyl 3-((3-(3-amino-1H-pyrazol-5-yl) naphthalen-2-yloxy)methyl) pyrrolidine-1-carboxylate: To a solution of tert-butyl 3-((3-(2-cyanoacetyl) naphthalen-2-yloxy)methyl) pyrrolidine-1-carboxylate (620 mg, 1.57 mmol, 1.0 eq) in EtOH (12 mL) was added hydrazine hydrate (1.18 g, 23.5 mmol, 15.0 eq) followed by dropwise addition of acetic acid (5 drops) and the resultant reaction mixture was kept for stirring at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (300 mg). LCMS: 409.1 [M+H]⁺

Step-5: Synthesis of tert-butyl 3-((3-(3-(5-cyanopyrazin-2-ylamino)-1H-pyrazol-5-yl) naphthalen-2-yloxy)methyl) pyrrolidine-1-carboxylate: To a solution of tert-butyl 3-(((3-(3-amino-1H-pyrazol-5-yl) naphthalen-2-yl)oxy)methyl) pyrrolidine-1-carboxylate (150 mg, 0.367 mmol, 1.0 eq) in EtOH (5.0 mL) was added DIPEA (0.17 mL, 0.918 mmol, 2.5 eq) followed by addition of 5-bromopyrazine-2-carbonitrile (135 mg, 0.734 mmol, 2.0 eq) and the reaction mixture was stirred at 90° C. for overnight. Product formation was confirmed by TLC and LCMS. The reaction mixture was concentrated under reduced pressure to get the crude. The crude by purified by flash chromatography by using 12 g RediSep® column and by eluting in 2% MeOH/DCM solvent system to obtain the desired compound (70 mg,). LCMS: 512.3 [M+H]⁺

Step-6: Synthesis of 5-(5-(3-(pyrrolidin-3-ylmethoxy) naphthalen-2-yl)-1H-pyrazol-3-ylamino) pyrazine-2-carbonitrile: To a solution of tert-butyl 3-((3-(3-(5-cyanopyrazin-2-ylamino)-1H-pyrazol-5-yl) naphthalen-2-yloxy)methyl) pyrrolidine-1-carboxylate (70 mg, 0.137 mmol, 1.0 eq) in DCM (5.0 mL), TFA (54.6 mg, 0.479 mmol, 3.5 eq) was added at 0° C. and the reaction mixture was stirred for 2 h. The reaction mixture was concentrated under reduced pressure to get the crude. The crude was purified by R-HPLC to yield desired compound (12 mg, formate salt). LCMS: 412.3 [M+1]+; 99.89 @ 220 nm, 99.86 @ 254 nm. 1H NMR (400 MHZ, DMSO-d6) δ 8.66 (s, 1H), 8.47 (s, 1H), 8.37 (s, 1H), 8.24 (s, 1H), 7.85 (dd, J=15.8, 8.1 Hz, 2H), 7.57-7.46 (m, 2H), 7.41 (t, J=7.4 Hz, 1H), 7.17 (s, 1H), 4.26 (dd, J=9.4, 6.2 Hz, 1H), 4.16 (t, J=8.7 Hz, 1H), 3.46-3.36 (m, 1H), 3.28-3.04 (m, 3H), 2.93-2.86 (m, 1H), 2.21 (dq, J=14.1, 7.3 Hz, 1H), 1.81 (dq, J=14.8, 7.6 Hz, 1H)

### Example-28: Synthesis of 5-(5-(3-(piperidin-3-ylmethoxy) naphthalen-2-yl)-1H-pyrazol-3-ylamino) pyrazine-2-carbonitrile (Compound 2.5)

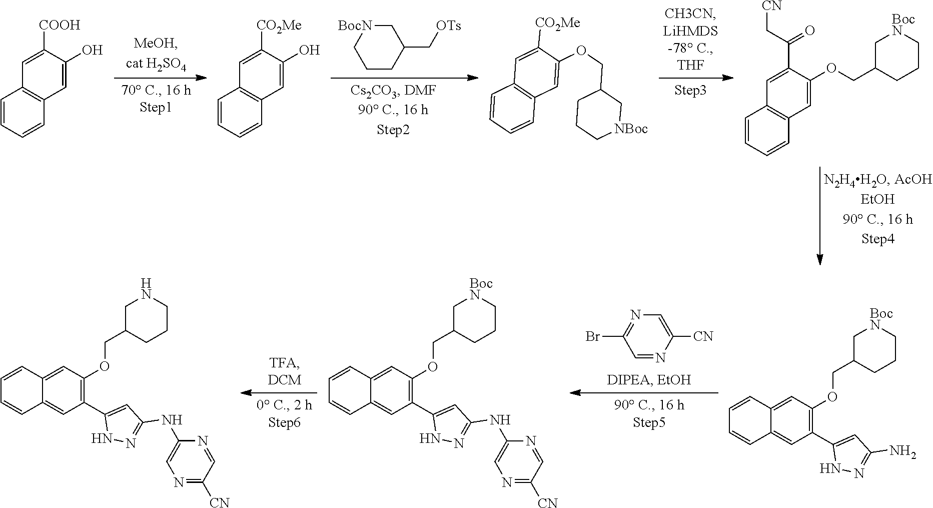

Step-1: Synthesis of methyl 3-hydroxy-2-naphthoate: Synthesized as described in WO2000009169A1.

Step-2: Synthesis of tert-butyl 3-((3-(methoxycarbonyl) naphthalen-2-yloxy)methyl) piperidine-1-carboxylate: To a solution of tert-butyl 3-(tosyloxymethyl) piperidine-1-carboxylate (2.0 g, 5.41 mmol, 1.0 eq) in DMF (40 mL), Cs₂CO₃ (4.41 g, 13.53 mmol, 2.5 eq) was added followed by addition of methyl 3-hydroxy-2-naphthoate (1.42 g, 7.04 mmol, 1.3 eq) and the reaction mixture was kept for stirring at 90° C. overnight. Reaction progress was monitored by TLC and LCMS. The reaction mixture was quenched by addition of ice water (50 mL) and extracted with ethyl acetate (3×25 mL). Collective organic layer was washed with brine (25 mL), dried over Na₂SO₄ and then concentrated under reduced pressure to get the crude. The crude was purified by flash chromatography by using 24 g RediSep® column and by eluting in 30% ethyl acetate: hexane solvent system to obtain the desired compound (1.3 g). LCMS: 300.3 [M+H−100]⁺; de-Boc peak

Step-3: Synthesis of tert-butyl 3-((3-(2-cyanoacetyl) naphthalen-2-yloxy)methyl) piperidine-1-carboxylate: To a solution of acetonitrile (7.0 mL) in THF (15 mL) was added LiHMDS (1M in THF) (8.7 mL, 8.7 mmol, 3.5 eq) at −78° C. and stirred for 2 h at same temperature. Then tert-butyl 3-((3-(methoxycarbonyl) naphthalen-2-yloxy)methyl) piperidine-1-carboxylate (1.0 g, 2.50 mmol, 1.0 eq) was added and the reaction mixture was stirred for 2 h. Product formation was confirmed by TLC and LCMS. The reaction mixture was quenched with water (20 mL) and extracted with ethyl acetate (3×15 mL). Collective organic layer was washed with brine (20 mL) and dried over Na₂SO₄ and then concentrated under reduced pressure to get the crude. The crude by purified by flash chromatography by using 12 g RediSep® column and by eluting in 30% ethyl acetate: hexane solvent system to obtain the desired compound (550 mg).LCMS: 309.4 [M+H−Boc]⁺

Step-4: Synthesis of tert-butyl 3-((3-(3-amino-1H-pyrazol-5-yl) naphthalen-2-yloxy)methyl) piperidine-1-carboxylate: To a solution of tert-butyl 3-((3-(2-cyanoacetyl) naphthalen-2-yloxy)methyl) piperidine-1-carboxylate (550 mg, 1.35 mmol, 1.0 eq) in EtOH (5.0 mL) was added hydrazine hydrate (1.01 mL, 20.2 mmol, 15.0 eq) followed by dropwise addition of acetic acid (0.1 mL) and the resultant reaction mixture was kept for stirring at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (250 mg). LCMS: 423.7 [M+H]⁺

Step-5: Synthesis of tert-butyl 3-((3-(3-(5-cyanopyrazin-2-ylamino)-1H-pyrazol-5-yl) naphthalen-2-yloxy)methyl) piperidine-1-carboxylate: To a solution of tert-butyl 3-((3-(3-amino-1H-pyrazol-5-yl) naphthalen-2-yloxy)methyl) piperidine-1-carboxylate (150 mg, 0.355 mmol, 1.0 eq) in EtOH (5.0 mL) was added DIPEA (0.19 mL, 1.07 mmol, 3.0 eq) followed by addition of 5-bromopyrazine-2-carbonitrile (78.3 mg, 0.426 mmol, 1.2 eq) and the reaction mixture was added and kept for stirring at 90° C.° C. for 6 h. Product formation was confirmed by TLC and LCMS. The reaction mixture was concentrated under reduced pressure to get the crude. The crude by purified by flash chromatography by using 12 g RediSep® column and by eluting in 2% MeOH/DCM solvent system to obtain the desired compound (110 mg). LCMS: 526.3 [M+H]⁺

Step-6: Synthesis of 5-(5-(3-(piperidin-3-ylmethoxy) naphthalen-2-yl)-1H-pyrazol-3-ylamino) pyrazine-2-carbonitrile: To a solution of tert-butyl 3-((3-(3-(5-cyanopyrazin-2-ylamino)-1H-pyrazol-5-yl) naphthalen-2-yloxy)methyl) piperidine-1-carboxylate (110 mg, 0.209 mmol, 1.0 eq) in DCM (5.0 mL), TFA (83.5 mg, 0.733 mmol, 3.5 eq) was added at 0° C. and kept for stirring for 2 h. The reaction mixture was concentrated under reduced pressure to get the crude. The crude was purified by R-HPLC to yield desired compound (15 mg). LCMS: 426.3 [M+H]+; UPLC: 99.86 @ 220 nm, 99.90 @ 254 nm. 1H NMR (400 MHZ, Methanol-d4) δ 8.56 (s, 1H), 8.53 (s, 1H), 8.48 (s, 1H), 8.10 (s, 1H), 7.86 (d, J=8.1 Hz, 1H), 7.82 (d, J=8.2 Hz, 1H), 7.54-7.45 (m, 1H), 7.45 (s, 1H), 7.40 (t, J=7.3 Hz, 1H), 7.04 (s, 1H), 4.27 (dd, J=9.8, 5.0 Hz, 1H), 4.17 (dd, J=9.7, 6.9 Hz, 1H), 3.58-3.49 (m, 1H), 3.38 (s, 1H), 2.93 (td, J=12.9, 12.3, 8.7 Hz, 2H), 2.44 (s, 1H), 2.09 (d, J=14.0 Hz, 1H), 2.03-1.97 (m, 1H), 1.87-1.71 (m, 1H), 1.65-1.51 (m, 1H).

### Example-29: Synthesis of 5-((5-(3-(2-aminoethoxy) naphthalen-2-yl)-1H-pyrazol-3-yl)amino) pyrazine-2-carbonitrile (Compound 2.6)

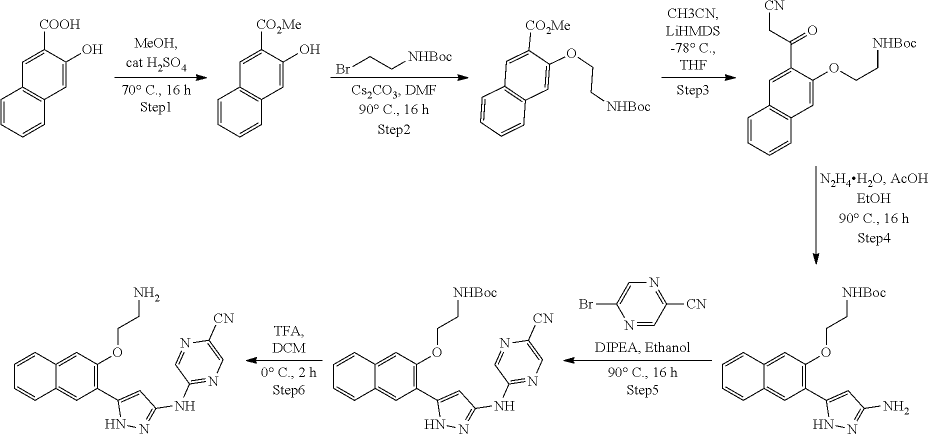

Step-1: Synthesis of methyl 3-hydroxy-2-naphthoate: Synthesized as described in WO2000009169 A1

Step-2: Synthesis of methyl 3-(2-(tert-butoxycarbonylamino) ethoxy)-2-naphthoate: To a solution of methyl 3-hydroxy-2-naphthoate (2.0 g, 9.89 mmol, 1.0 eq) in DMF (40 mL), Cs₂CO₃ (4.83 g, 14.84 mmol, 1.5 eq) was added followed by addition of tert-butyl (2-bromoethyl) carbamate (2.66 g, 11.87 mmol, 1.2 eq) and the reaction mixture was kept for stirring at 90° C. overnight. Reaction progress was monitored by TLC and LCMS. The reaction mixture was quenched by addition of ice water (50 mL) and extracted with ethyl acetate (3×25 mL). Collective organic layer was washed with brine (25 mL), dried over Na₂SO₄ and then concentrated under reduced pressure to get the crude. The crude was purified by flash chromatography by using 24 g RediSep® column and by eluting in 30% ethyl acetate: hexane solvent system to obtain the desired compound (620 mg). LCMS: 246.3 [M+H−100]⁺; de-Boc peak

Step-3: Synthesis of tert-butyl (2-((3-(2-cyanoacetyl) naphthalen-2-yl)oxy)ethyl) carbamate: To a solution of acetonitrile (10 mL) in THF (8 mL) was added LiHMDS (1M in THF) (6.28 mL, 6.28 mmol, 3.5 eq) at −78° C. and stirred for 2 h at same temperature. Then methyl 3-(2-(tert-butoxycarbonyl)aminoethoxy)-2-naphthoate (620 mg, 1.8 mmol, 1.0 eq) was added and the reaction mixture was stirred for 2 h. Product formation was confirmed by TLC and LCMS. The reaction mixture was quenched with water (20 mL) and extracted with ethyl acetate (3×15 mL). Collective organic layer was washed with brine (20 mL), dried over Na₂SO₄ and then concentrated under reduced pressure to get the crude. The crude by purified by flash chromatography by using 12 g RediSep® column and by eluting in 30% ethyl acetate: hexane solvent system to obtain the desired compound (320 mg).LCMS: 255.3 [M+H−100]⁺

Step-4: Synthesis of tert-butyl 2-(3-(3-amino-1H-pyrazol-5-yl) naphthalen-2-yloxy)ethylcarbamate: To a solution of tert-butyl 2-(3-(2-cyanoacetyl) naphthalen-2-yloxy)ethylcarbamate (300 mg, 0.847 mmol, 1.0 eq) in EtOH (5.0 mL) was added hydrazine hydrate (635 mg, 12.71 mmol, 15.0 eq) followed by dropwise addition of acetic acid (5 drops) and the resultant reaction mixture was kept for stirring at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (60 mg). LCMS: 369.4 [M+H]⁺

Step-5: Synthesis of tert-butyl 2-(3-(3-(5-cyanopyrazin-2-yl)amino)-1H-pyrazol-5-yl) naphthalen-2-yloxy)ethylcarbamate: To a solution of tert-butyl 2-(3-(3-amino-1H-pyrazol-5-yl) naphthalen-2-yloxy)ethylcarbamate (60 mg, 0.163 mmol, 1.0 eq) in EtOH (5.0 mL) was added DIPEA (0.07 mL, 0.407 mmol, 2.5 eq) followed by addition of 5-bromopyrazine-2-carbonitrile (59.9 mg, 0.326 mmol, 2.0 eq) and the reaction mixture was stirred at 90° C. for overnight. Product formation was confirmed by TLC and LCMS. The reaction mixture was concentrated under reduced pressure to get the crude. The crude by purified by flash chromatography by using 12 g RediSep® column and by eluting in 2% MeOH/DCM solvent system to obtain the desired compound (40 mg). LCMS: 472.3 [M+H]⁺

Step-6: Synthesis of 5-(5-(3-(2-aminoethoxy) naphthalen-2-yl)-1H-pyrazol-3-ylamino) pyrazine-2-carbonitrile: To a solution of tert-butyl 2-(3-(3-(5-cyanopyrazin-2-ylamino)-1H-pyrazol-5-yl) naphthalen-2-yloxy)ethylcarbamate (40 mg, 0.084 mmol, 1.0 eq) in DCM (4.0 mL), TFA (34 mg, 0.297 mmol, 3.5 eq) was added at 0° C. and kept for stirring for 2 h. The reaction mixture was concentrated under reduced pressure to get the crude. The crude was purified by R-HPLC to yield desired compound (4 mg, formate salt). LCMS: 372.3 [M+H]+; UPLC: 99.03 @ 220 nm, 98.98 @ 254 nm. ¹HNMR (400 MHZ, DMSO-d6) δ 8.67 (s, 1H), 8.52 (s, 1H), 8.32 (s, 1H), 8.24 (s, 1H), 7.91 (d, J=8.2 Hz, 1H), 7.85 (d,J=8.1 Hz, 1H), 7.58-7.47 (m, 2H), 7.41 (t, J=7.5 Hz, 1H), 7.12 (s, 1H), 4.35 (s, 2H), 3.27 (s, 2H).

### Example-30: Synthesis of 5-(5-(3-(3-aminopropoxy) naphthalen-2-yl)-1H-pyrazol-3-ylamino) pyrazine-2-carbonitrile (Compound 2.7)

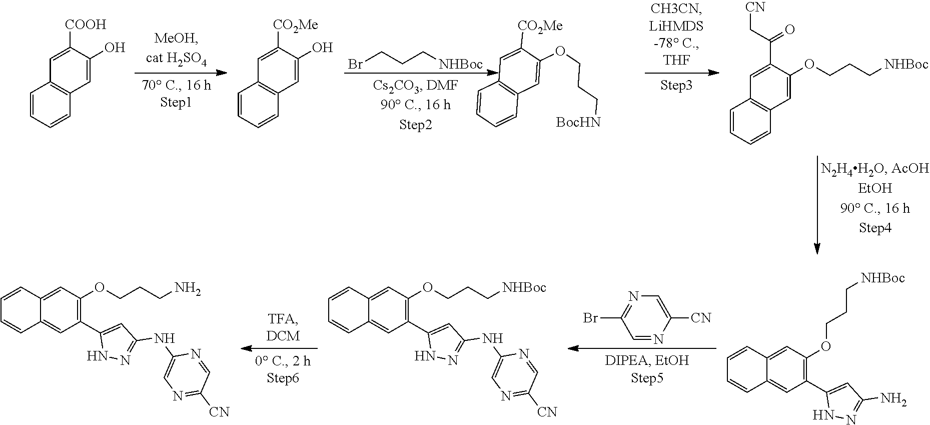

Step-1: Synthesis of methyl 3-hydroxy-2-naphthoate: Synthesized as described in WO2000009169A1.

Step-2: Synthesis of methyl 3-(3-(tert-butoxycarbonylamino) propoxy)-2-naphthoate: To a solution of methyl 3-hydroxy-2-naphthoate (2.5 g, 12.36 mmol, 1.0 eq) in DMF (40 mL), Cs₂CO₃ (10.0 g, 30.9 mmol, 2.5 eq) was added followed by addition of tert-butyl 3-bromopropylcarbamate (3.83 g, 16.07 mmol, 1.3 eq) and the reaction mixture was kept for stirring at 90° C. overnight. Reaction progress was monitored by TLC and LCMS. The reaction mixture was quenched by addition of ice water (50 mL) and extracted with ethyl acetate (3× 30 mL). Collective organic layer was washed with brine (50 mL), dried over Na₂SO₄ and then concentrated under reduced pressure to get the crude. The crude was purified by flash chromatography by using 24 g RediSep® column and by eluting in 30% ethyl acetate: hexane solvent system to obtain the desired compound (2.0 g). LCMS: 260.4 [M+H−100]⁺; de-Boc peak

Step-3: Synthesis of tert-butyl (3-(3-(2-cyanoacetyl) naphthalen-2-yloxy) propyl) carbamate: To a solution of acetonitrile (10.0 mL) in THF (30 mL) was added LiHMDS (1M in THF) (16.7 mL, 16.7 mmol, 3.0 eq) at −78° C. and stirred for 2 h at same temperature. Then methyl 3-(3-(tert-butoxycarbonylamino) propoxy)-2-naphthoate (2.0 g, 5.56 mmol, 1.0 eq) was added and the reaction mixture was stirred for 2 h. Product formation was confirmed by TLC and LCMS. The reaction mixture was quenched with water (50 mL) and extracted with ethyl acetate (3×25 mL). Collective organic layer was washed with brine (20 mL) and dried over Na₂SO₄ and then concentrated under reduced pressure to get the crude. The crude by purified by flash chromatography by using 12 g RediSep® column and by eluting in 30% ethyl acetate: hexane solvent system to obtain the desired compound (1.1 g). ¹H NMR (400 MHZ, DMSO-d6) δ 8.27 (s, 1H), 7.95 (d, J=8.2 Hz, 1H), 7.84 (d, J=8.4 Hz, 1H), 7.56 (t, J=7.5 Hz, 1H), 7.45 (s, 1H), 7.41 (t, J=7.5 Hz, 1H), 6.90 (t, J=5.7 Hz, 1H), 4.14 (t, J=6.0 Hz, 2H), 3.86 (s, 2H), 3.16 (q, J=6.5 Hz, 2H), 1.90 (p, J=6.4 Hz, 2H), 1.37 (s, 9H).

Step-4: Synthesis of tert-butyl 3-(3-(3-amino-1H-pyrazol-5-yl) naphthalen-2-yloxy) propylcarbamate: To a solution of tert-butyl 3-(3-(2-cyanoacetyl) naphthalen-2-yloxy) propylcarbamate (500 mg, 1.36 mmol, 1.0 eq) in EtOH (5.0 mL) was added hydrazine hydrate (1.02 mL, 20.36 mmol, 15.0 eq) followed by dropwise addition of acetic acid (0.1 mL) and the resultant reaction mixture was kept for stirring at 90° C. overnight. Product formation was confirmed by TLC and LCMS. The reaction mixture was concentrated under reduced pressure to get the crude. The crude was purified by flash chromatography by using 12 g RediSep® column and by eluting in 2% MeOH/DCM solvent system to obtain the desired compound (130 mg). LCMS: 383.4 [M+H]⁺

Step-5: Synthesis of tert-butyl 3-(3-(3-(5-cyanopyrazin-2-ylamino)-1H-pyrazol-5-yl) naphthalen-2-yloxy) propylcarbamate: To a solution of tert-butyl 3-(3-(3-amino-1H-pyrazol-5-yl) naphthalen-2-yloxy) propylcarbamate (130 mg, 0.339 mmol, 1.0 eq) in EtOH (5.0 mL) was added DIPEA (0.15 mL, 0.849 mmol, 2.5 eq) followed by addition of 4-bromo-1-naphthonitrile (125 mg, 0.670 mmol, 2.0 eq) and the reaction mixture was added and kept for stirring at 90° C. for 6 h. Product formation was confirmed by TLC and LCMS. The reaction mixture was concentrated under reduced pressure to get the crude. The crude by purified by flash chromatography by using 12 g RediSep® column and by eluting in 2% MeOH/DCM solvent system to obtain the desired compound (80 mg). LCMS: 486.3 [M+H]⁺

Step-6: Synthesis of 5-(5-(3-(3-aminopropoxy) naphthalen-2-yl)-1H-pyrazol-3-ylamino) pyrazine-2-carbonitrile: To a solution of tert-butyl 3-(3-(3-(5-cyanopyrazin-2-ylamino)-1H-pyrazol-5-yl) naphthalen-2-yloxy) propylcarbamate (80 mg, 0.164 mmol, 1.0 eq) in DCM (5.0 mL), TFA (65.8 mg, 0.577 mmol, 3.5 eq) was added at 0° C. and kept for stirring for 2 h. The reaction mixture was concentrated under reduced pressure to get the crude. The crude was purified by R-HPLC to yield desired compound (15 mg). LCMS: 386.3 [M+H]+; UPLC: 99.73 @ 220NM; 99.63@ 254NM; 1HNMR (400 MHZ, DMSO-d6) δ 8.67 (s, 1H), 8.47 (s, 1H), 8.39 (s, 1H), 8.22 (s, 1H), 7.88 (d, J=8.3 Hz, 1H), 7.83 (d, J=8.2 Hz, 1H), 7.49 (d, J=7.2 Hz, 2H), 7.40 (t, J=7.5 Hz, 1H), 7.14 (s, 1H), 4.29 (t, J=5.9 Hz, 2H), 3.06 (t, J=7.1 Hz, 2H), 2.15 (p,J=6.5 Hz, 2H).

### Biological Examples

### Example-31: CHK-1% Inhibition Kinase Assay

Inhibition of CHK-1 kinase or percent inhibition at defined concentrations was determined by TR-FRET assay. Kinase reactions were performed in a 20 μL volume in low-volume 384-well plates (Cat #Corning 4511). The concentration of substrate Fluorescein-CREBtide was maintained at 0.2 μM in the assay, and the kinase reaction buffer consisted of 50 mM HEPES pH 7.5, 0.01% BRIJ-35, 10 mM MgCl2, 1 mM EGTA. Serially diluted compounds (3-fold) in DMSO (0.5% in final reaction) were incubated with cocktail of CHK-1 kinase (6 nM; Cat #PR3959B, ThermoFisher), Fluorescein-CREBtide (0.2 μM; Cat #PV3508, ThermoFisher), ATP (15 μM; Cat #A1852, Sigma), and kinase reaction buffer. After 1 hour kinase reactions at room temperature, 10 μL of Detection mix (8 nM LanthaScreen® Tb-CREB [pSer133] antibody, Cat #PV3542, ThermoFisher with 20 mM EDTA in TR-FRET Buffer, Cat #PV3574, ThermoFisher) was added, and the plate was read with a plate reader (Synergy™ NEO; Biotek) for TR-FRET.

The resulting TR-FRET emission ratio was used to calculate the percent inhibition by normalizing to the control using the following formula:

$$[ \% Inhibition = 1 - \{ ( \% {Phosphorylation}_{sample} / \% {Phosphorylation}_{control ( 0 \% Inhibition )} \} * 100 ]$$

The results of % inhibition of CHK1 enzyme at 30 nM and 300 nM are shown in Table-B1

TABLE B1

| Compound No. | % Inhibition at 30 nM | % Inhibition at 300 nM |
|---|---|---|
| Compound 1.1 | 49 | 88 |
| Compound 1.2 | 36 | 85 |
| Compound 1.3 | 50 | 97 |
| Compound 1.4 | 7 | 54 |
| Compound 1.5 | 63 | 89 |
| Compound 1.6 | 71 | 91 |
| Compound 1.7 | 36 | 82 |
| Compound 1.8 | 51 | 64 |
| Compound 1.9 | 33 | 31 |
| Compound 1.10 | 44 | 65 |
| Compound 1.11 | 12 | 36 |
| Compound 1.12 | 21 | 30 |
| Compound 1.13 | 15 | 32 |
| Compound 1.14 | 37 | 60 |
| Compound 1.15 | 39 | 79 |
| Compound 1.16 | 9 | 75 |
| Compound 1.17 | 69 | 95 |
| Compound 1.18 | 4 | 28 |
| Compound 1.19 | 15.1 | 50.5 |
| Compound 1.20 | 60.2 | 91.9 |
| Compound 1.21 | 78.7 | 95.5 |
| Compound 1.22 | 13.7 | 11.0 |
| Compound 1.23 | 82.0 | 99.0 |
| Compound 2.1 | 27 | 91 |
| Compound 2.2 | 80 | 96 |
| Compound 2.3 | 41 | 81 |
| Compound 2.4 | 70 | 97 |
| Compound 2.5 | 72 | 96 |
| Compound 2.6 | 61 | 90 |
| Compound 2.7 | 95 | 100 |

The results of CHK1 IC₅₀ are shown in Table-B2

TABLE B2

| Compound No. | CHK1 IC₅₀ (nM) |
|---|---|
| Compound 1.1 | 67.7 |
| Compound 1.2 | 13.3 |
| Compound 1.3 | 7.77 |
| Compound 1.4 | 86.4 |
| Compound 1.5 | 7.82 |
| Compound 1.6 | 12.3 |
| Compound 1.8 | 76.6 |
| Compound 1.9 | >10000 |
| Compound 2.1 | 2.46 |
| Compound 2.2 | 4.14 |
| Compound 2.3 | 8.15 |
| Compound 2.4 | 2.34 |
| Compound 2.5 | 4.36 |
| Compound 2.6 | 15.7 |
| Compound 2.7 | 0.933 |

### Example-32: Microsomal Stability Assay

Incubation mixture contained 0.2 mg/mL microsomes, 1 mmol NADPH, and test compound at 1 μM in 100 mmol potassium phosphate buffer at pH 7.4. The mixtures were incubated at 37° C. for 5, 15, and 30 minutes. Aliquots of the mixtures were taken at 0, 5, 15 and 30 minutes and quenched with chilled Acetonitrile:Methanol (50:50) containing internal standard. Following completion of incubation, samples were centrifuged at 4,000 rpm at 4° C. The supernatant was analyzed by LC-MS/MS and half-life, as well as intrinsic clearance (Clint), were calculated.

The results of Microsomal stability assay are provided in the Table-B3.

TABLE B3

| | Clint HLM | Clint MLM | Clint RLM |
|---|---|---|---|
| Compound No. | (μl/min/mg) | (μl/min/mg) | (μl/min/mg) |
| Compound 1.1 | 32 | 100 | 58 |
| Compound 1.2 | 45 | 39 | 27 |
| Compound 1.3 | 47 | 68 | 61 |
| Compound 1.4 | 52 | 66 | 27 |
| Compound 1.5 | 78 | 77 | 70 |
| Compound 1.6 | 63 | 63 | 118 |
| Compound 1.7 | 75 | 114 | 118 |
| Compound 1.8 | 202 | 481 | 104 |
| Compound 1.9 | 159 | 83 | 85 |
| Compound 1.10 | 80 | 243 | 49 |
| Compound 1.11 | 140 | 382 | 79 |
| Compound 1.12 | 478 | 473 | 266 |
| Compound 1.13 | 86 | 221 | 84 |
| Compound 1.14 | 34 | 56 | 23 |
| Compound 1.15 | 12 | 25 | 33 |
| Compound 1.16 | 117 | 141 | 138 |
| Compound 1.17 | 26 | 50 | 43 |
| Compound 1.18 | 145 | 297 | 130 |
| Compound 1.19 | 183 | 239 | 88 |
| Compound 1.20 | 47 | 86 | 46 |
| Compound 1.21 | 20 | 18 | 37 |
| Compound 1.22 | 169 | 313 | 142 |
| Compound 1.23 | 37 | 52 | 50 |
| Compound 2.1 | 25 | 101 | 36 |
| Compound 2.2 | 21 | 153 | 45 |
| Compound 2.3 | 26 | 85 | 39 |
| Compound 2.4 | 33 | 142 | 94 |
| Compound 2.5 | 14 | 111 | 124 |
| Compound 2.6 | 3 | 153 | 58 |
| Compound 2.7 | 6 | 118 | 44 |

HLM: human liver microsome;
MLM: mouse liver microsome;
RLM: rat liver microsome

### Example-33: Kinetic Solubility Assay

Test articles were serially diluted in DMSO from concentration range of 10 mM to 0.078 mM in 96 well v-bottom dilution plate (#3363, Corning). 1 μL of test article from each well was transferred to 96 well flat bottom clear plates (#655101, Greiner) containing 99 μL of PBS at pH-7.4, which makes the test drug final concentration range from 100 μM to 0.78 μM. Samples were incubated for 1 hour at 37° C. followed by measurement of light scattering at 625 nm with a laser based micro plate Nephelometer (Nephlostar Plus, BMG Labtech). Solubility concentration (μM) was then calculated by segmental regression with Neplometer as Readout.

The results of kinetic solubility assay are provided in Table-B4.

TABLE B4

| Compound No. | Kinetic solubility (μM) |
|---|---|
| Compound 1.1 | 44.0 |
| Compound 1.2 | 45.6 |
| Compound 1.3 | 100 |
| Compound 1.4 | 100 |
| Compound 1.5 | 41.8 |
| Compound 1.6 | 42.8 |
| Compound 1.7 | 39.2 |
| Compound 1.8 | 32 |
| Compound 1.9 | 100 |
| Compound 1.10 | 30.5 |
| Compound 1.11 | 14 |
| Compound 1.12 | 38.4 |
| Compound 1.13 | 11.9 |
| Compound 1.14 | 39.6 |
| Compound 1.15 | 100 |
| Compound 1.16 | 38.2 |
| Compound 1.17 | 48 |
| Compound 1.18 | 48 |
| Compound 1.19 | 27.6 |
| Compound 1.20 | 29.4 |
| Compound 1.21 | 18.8 |
| Compound 1.22 | 28.4 |
| Compound 1.23 | 46.6 |
| Compound 2.1 | 34.7 |
| Compound 2.2 | 41.8 |
| Compound 2.3 | 39.7 |
| Compound 2.4 | 7.77 |
| Compound 2.5 | 41.6 |
| Compound 2.6 | 18.8 |
| Compound 2.7 | 21.3 |

### Example-34: Caco-2 Cell Permeability Assay

After thawed, Caco-2 cells were cultured in the medium containing 10% FBS, 1% Sodium pyruvate, 1% NEAA and 1% Glutamax MEM, then were placed in the incubator containing 5% CO₂ at 37° C., and the culture medium then was replaced every 2-3 days. When the confluence reached 80%, the subculture was carried out. Add 5 mL of 1 M HEPES to 500 mL of HBSS for preparing transport buffer (pH 7.4), and the final concentration of HEPES was 10 mM. Before the transport study (day 21-28), take out the culture medium from the cell culture plate, wash it twice with preheated HBSS buffer (10 mM HEPES, pH 7.4), and add 100 μL HBSS buffer to each well. After incubation at 37° C. for 30 minutes, Teer value was measured by cell resistance meter (Millicell-ERS2) to confirm the integrity of monolayer cells. Discard the HBSS buffer, transfer the upper plate containing the cell monolayer membrane to the corresponding receiving plate, add the test compound solution or buffer to the top (side A) and the bottom (side B) respectively, put the cell plate into the corresponding 96 well receiving plate, and add the prepared positive control compound, test compound or HBSS buffer (with 0.5% BSA) on side A or side B of the filter membrane of Transwell cell plate according to the following groups, incubate cell plates at 37° C. When incubated for 5 minutes, 8 μL sample solutions were taken from the side A and the side B respectively and add 72 μL transport buffer (diluted 10 times) as TO of the initial solution. After 120 minutes of incubation, collect the samples at both ends of the filter membrane of Transwell cell plate; take 80 μL sample solution from the receiving side as the sample on the receiving side after transport, meanwhile take 8 μL sample solution from the administration side and then mix with 72 μL transport buffer, the well-mixed solution was used as the sample on the administration side after transport. After collecting bilateral samples, tap gently, discard the remaining solution in the cell plate, wash the cell plate with transport buffer; and to each well, add 100 μL acetonitrile solution containing internal standard working solution to lyse cells; After mixing well by blowing up and down, take 80 μL per well cell lysate as the sample after cell lysis. After collecting all samples in the transport system (TO sample from receiving side and administration side), 160 μL IS/ACN solution was added to each well, 80 μL IS/ACN solution was added to the collected lysate sample, and then seal it. Shake for 10 minutes and store the sample plate at −20° C. before LC-MS/MS analysis.

The following equation are used to calculate the apparent permeability coefficient (Papp, cm/s), the efflux ratio (ER), the solution recovery (% Recovery) and the total recovery (% Total Recovery).

$${P}_{app} = \frac{{V}_{recevier}}{Area \times Time} \times \frac{{[ drug ]}_{recevier}}{{[ drug ]}_{initial}}$$

$$Efflux Ratio = \frac{{P}_{app} ( B to A )}{{P}_{app} ( A to B )}$$

$$\% Recovery = \frac{{[ drug ]}_{receiver} \times {V}_{receiver} + {[ drug ]}_{donor} \times {V}_{donor}}{{[ drug ]}_{initial} \times {V}_{donor}} \times 100$$

$$\% Total Recovery = \frac{{[ drug ]}_{receiver} \times {V}_{receiver} + {[ drug ]}_{lyste} \times {V}_{lysate} + {[ drug ]}_{donor} \times {V}_{donor}}{{[ drug ]}_{inital} \times {V}_{donor}} \times 100$$

Where,
V_{receiver} is the volume of solution at the receiving end (0.25 mL for A to B and 0.1 mL for B to A);
V_{donor} is the volume of the solution at the administration end (0.1 mL for A to B and 0.25 mL for B to A);
V_{lysate} is the volume of cell lysate (0.1 mL);
Area is the relative surface area of the cell monolayer (0.0804 cm²);
Time is the incubation time (7200 s);
[drug]_{receiver} is the drug concentration (nM) at the receiving end or the control compound;
[drug]_{donor} is the concentration of drug or control compound at the administration end (nM);
[drug]_{lysate} is the concentration of drug or control compound in cell lysate (nM);
[drug]ᵢₙᵢₜᵢₐₗ is the initial concentration of the test or control compound (nM).

TABLE B5

| | Summary | | | | | | |
|---|---|---|---|---|---|---|---|
| | | | | | | Recovery % | |
| | Papp | | | | | with Cell | |
| | (10⁻⁶ cm/s) | | | Recovery % | | Lysate | |
| Compound | A to | B to | Efflux | A to | B to | A to | B to |
| ID | B | A | Ratio | B | A | B | A |
| Compound 1.3 | 3.40 | 11.16 | 3.28 | 54.7 | 103.0 | 89.0 | 107.2 |
| Compound 1.2 | 5.35 | 9.75 | 1.82 | 67.7 | 103.2 | 93.9 | 108.2 |
| Compound 1.1 | 7.00 | 8.70 | 1.24 | 69.0 | 102.2 | 94.1 | 107.2 |

1. A compound of formula (I): 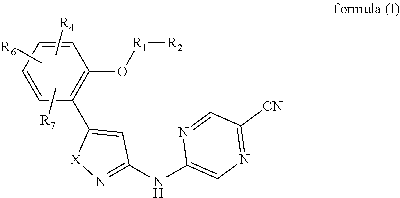
wherein,
X is selected from the group consisting of O or NH;
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄, R₆ and R₇ are independently -A-R₅ or absent;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R³ is selected from the group consisting of H, or C₁-C₃ alkyl; n is 0, 1 or 2;
or R₄ and R₆, together with the carbons to which they are attached, form a 5-, 6-, 7-, or, 8-membered ring, wherein said ring is partially or fully unsaturated and substituted or unsubstituted;
or a pharmaceutically acceptable salt thereof.

2. The compound of claim 1, wherein formula (I) is 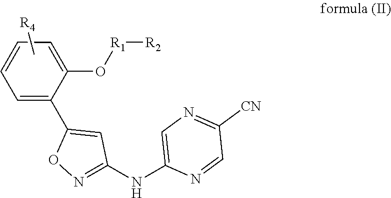
wherein,
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄ is-A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) saturated or unsaturated monocyclic 5-6 membered heterocyclyl, wherein the heterocyclyl contains 1-3 heteroatoms independently selected from N(R₃)ₙ, O or S; optionally each R₃ is selected from the group consisting of H, or C₁-C₃ alkyl; n is 0, 1 or 2;
or a pharmaceutically acceptable salt thereof.

3. The compound of claim 2, wherein, formula (II) is selected from the group consisting of:

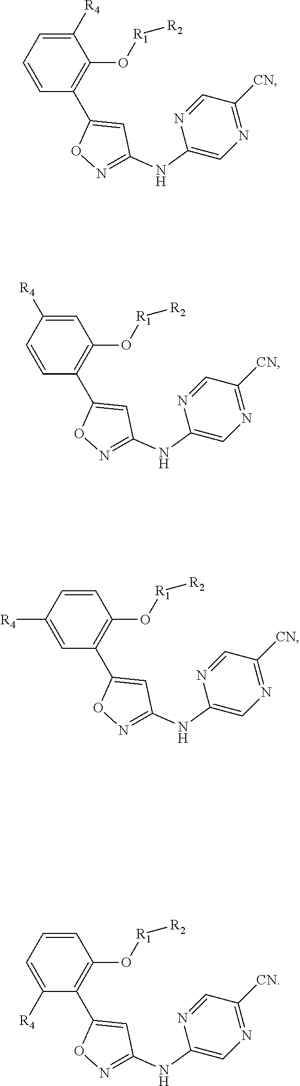

4. The compound of claim 2 or 3, wherein,
R₄ is -A-R₅;
A is selected from:
(i) a bond; or
(ii) a saturated chain of 2 to 4 chain members in length containing at least one carbon atom chain member, at least one heteroatom chain member selected from nitrogen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of
(i) H, or
(ii) saturated or unsaturated monocyclic 6 membered heterocyclyl, wherein the heterocyclyl contains 1-2 heteroatoms independently selected from N(R₃)ₙ, optionally each R₃ is selected from the group consisting of H, or C₁-C₃ alkyl, n is 0, 1 or 2.

5. The compound of anyone of claims 2 to 4, wherein,
R₄ is -A-R₅;
A is selected from:
(i) a bond; or
(ii)-(CH₂)₁₋₄, —NH—(CH₂)₁₋₃;
R₅ is selected from the group consisting of
(i) H, or

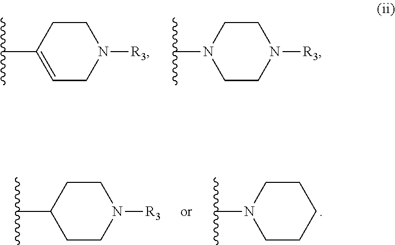

6. The compound of claim 1, wherein formula (I) is 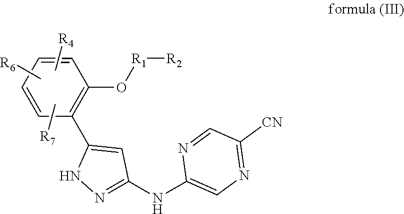
wherein,
R₁ is selected from the group consisting of C₁-C₆ alkyl, C₁-C₆ alkylene, —C₃-C₈ cycloalkyl or absent;
R₂ is selected from the group consisting of H, —NH₂, unsaturated or saturated monocyclic, bicyclic or spirocyclic 4- to 10-membered heterocyclyl, wherein the heterocyclyl contains 1-2 heteroatoms independently selected from N(R₃)ₙ, and O;
R₄, R₆ and R₇ are independently -A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R³ is selected from the group consisting of H, or C₁-C₃ alkyl; n is 0, 1 or 2;
or R₄ and R₆, together with the carbons to which they are attached, form a 5-, 6-, 7-, or, 8-membered ring, wherein said ring is partially or fully unsaturated and substituted or unsubstituted;
or a pharmaceutically acceptable salt thereof.

7. The compound of claim 6, wherein, formula (III) is 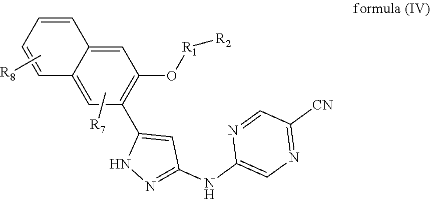
wherein R₈ is -A-R₅;
A is selected from the group consisting of:
(i) a bond;
(ii) CH₂;
(iii) a saturated chain of 2 to 10 chain members in length containing at least one carbon atom chain member, at least one heteroatom chain member selected from nitrogen and oxygen, and optionally one or more further carbon atom chain members and/or heteroatom chain members selected from nitrogen, oxygen, sulphur, sulphinyl and sulphonyl; the saturated chain being optionally substituted with one or more substituents selected from =O, C₁₋₄ hydrocarbyl and fluorine wherein two hydrocarbyl substituents on the same carbon atom may optionally link to form a ring of three to five ring members;
R₅ is selected from the group consisting of:
(i) H,
(ii) CH₂,
(iii) saturated or unsaturated monocyclic 5-6 membered heterocyclyl, wherein the heterocyclyl contains 1-3 heteroatoms independently selected from N(R₃)ₙ, O or S; optionally each R₃ is selected from the group consisting of H, or C₁-C₃ alkyl; n is 0, 1 or 2,
or a pharmaceutically acceptable salt thereof.

8. The compound of claim 7, wherein, formula (IV) is 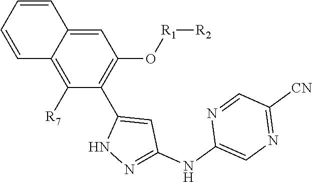
or a pharmaceutically acceptable salt thereof.

9. The compound of anyone of claims 1 to 8, wherein,
R₁ is selected from the group consisting of C₁-C₃ alkyl, C₁-C₄ alkylene, —C₃-C₆ cycloalkyl or absent;
R₂ is selected from the group consisting of H, —NH₂, unsaturated or saturated monocyclic, or spirocyclic 4- to 6-membered heterocyclyl, wherein the heterocyclyl contains 1-2 heteroatoms independently selected from N(R₃)ₙ, and O;
or a pharmaceutically acceptable salt thereof.

10. The compound of anyone of claims 1 to 9, wherein R¹ is selected from the group consisting methyl, ethyl, n-propyl, isopropyl, —CH₂—, —CH₂CH₂—, —CH₂CH₂CH₂—, —CH₂CH₂CH₂CH₂—, cyclopropyl, cyclobutyl, cyclopentyl, cyclohexyl, or absent.

11. The compound of anyone of claims 1 to 10, wherein,
R₂ is selected from the group consisting of H, —NH₂,

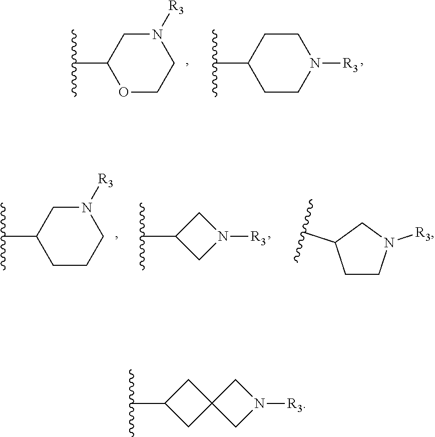

12. The compound of anyone of claims 1 to 11, wherein R₃ is selected from the group consisting of H, methyl, ethyl, n-propyl, isopropyl, n-butyl, t-butyl, isobutyl, sec-butyl, n-pentyl, isopentyl, neopentyl, n-hexyl, 2-methylpentyl, 3-methylpentyl, 2,3-dimethylbutyl, or 2,2-dimethylbutyl.

13. The compound of anyone claim of 1 to 12, wherein the compound is selected from the group consisting of: 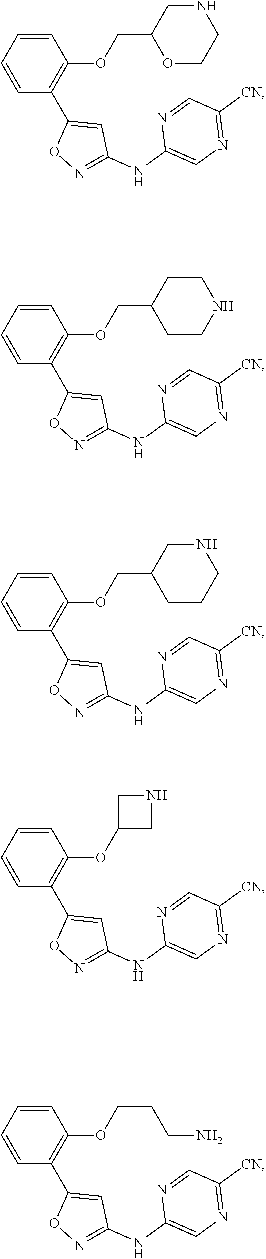 [SMILES: N#Cc1cnc(Nc2cc(-c3ccccc3OCC3CNCCO3)on2)cn1 ; N#Cc1cnc(Nc2cc(-c3ccccc3OCC3CCNCC3)on2)cn1 ; N#Cc1cnc(Nc2cc(-c3ccccc3OCC3CCCNC3)on2)cn1 ; N#Cc1cnc(Nc2cc(-c3ccccc3OC3CNC3)on2)cn1 ; N#Cc1cnc(Nc2cc(-c3ccccc3OCCCN)on2)cn1] 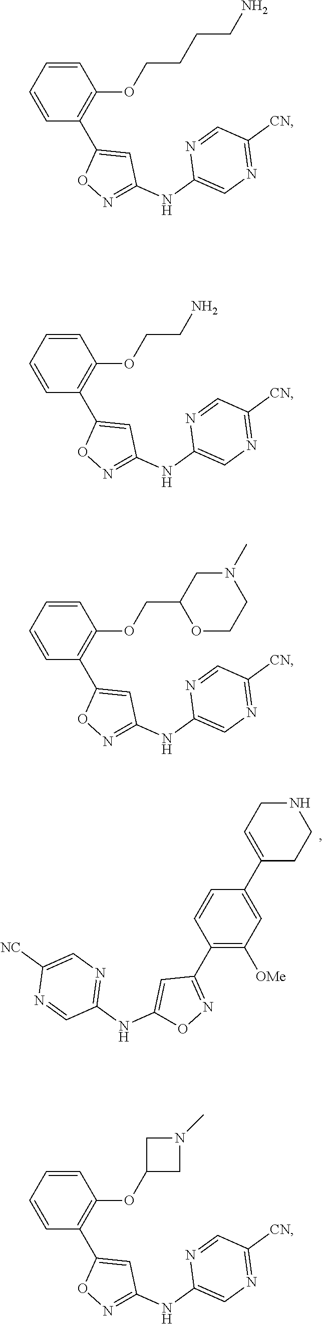 [SMILES: N#Cc1cnc(Nc2cc(-c3ccccc3OCCCCN)on2)cn1 ; N#Cc1cnc(Nc2cc(-c3ccccc3OCCN)on2)cn1 ; CN1CCOC(COc2ccccc2-c2cc(Nc3cnc(C#N)cn3)no2)C1 ; COc1cc(C2=CCNCC2)ccc1-c1cc(Nc2cnc(C#N)cn2)on1 ; CN1CC(Oc2ccccc2-c2cc(Nc3cnc(C#N)cn3)no2)C1] 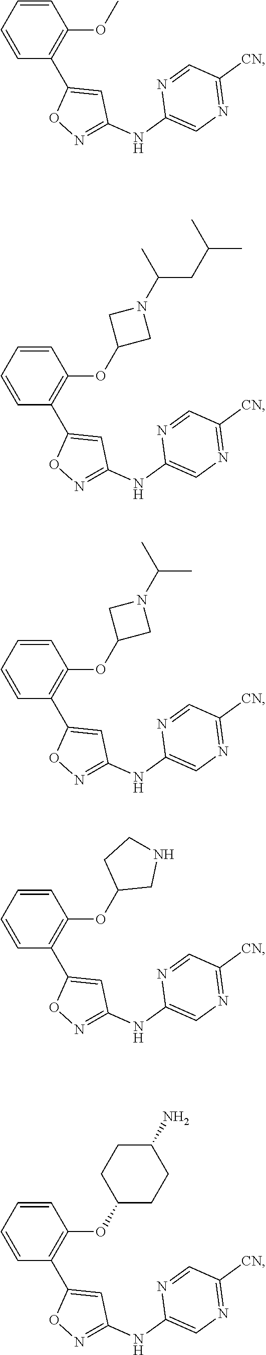 [SMILES: COc1ccccc1-c1cc(Nc2cnc(C#N)cn2)no1 ; CC(C)CC(C)N1CC(Oc2ccccc2-c2cc(Nc3cnc(C#N)cn3)no2)C1 ; CC(C)N1CC(Oc2ccccc2-c2cc(Nc3cnc(C#N)cn3)no2)C1 ; N#Cc1cnc(Nc2cc(-c3ccccc3OC3CCNC3)on2)cn1 ; N#Cc1cnc(Nc2cc(-c3ccccc3O[C@H]3CC[C@@H](N)CC3)on2)cn1] 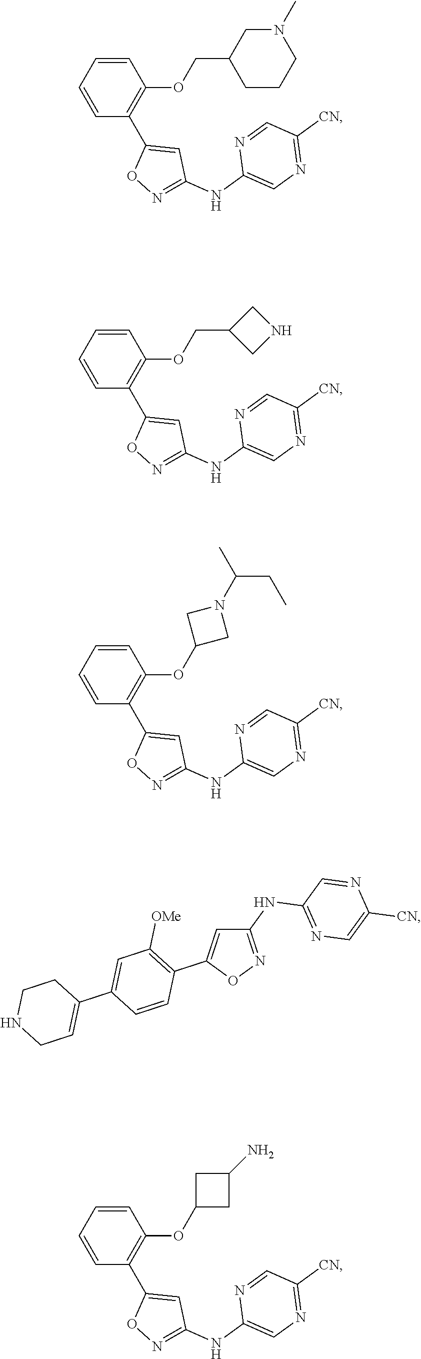 [SMILES: CN1CCCC(COc2ccccc2-c2cc(Nc3cnc(C#N)cn3)no2)C1 ; N#Cc1cnc(Nc2cc(-c3ccccc3OCC3CNC3)on2)cn1 ; CCC(C)N1CC(Oc2ccccc2-c2cc(Nc3cnc(C#N)cn3)no2)C1 ; COc1cc(C2=CCNCC2)ccc1-c1cc(Nc2cnc(C#N)cn2)no1 ; N#Cc1cnc(Nc2cc(-c3ccccc3OC3CC(N)C3)on2)cn1] 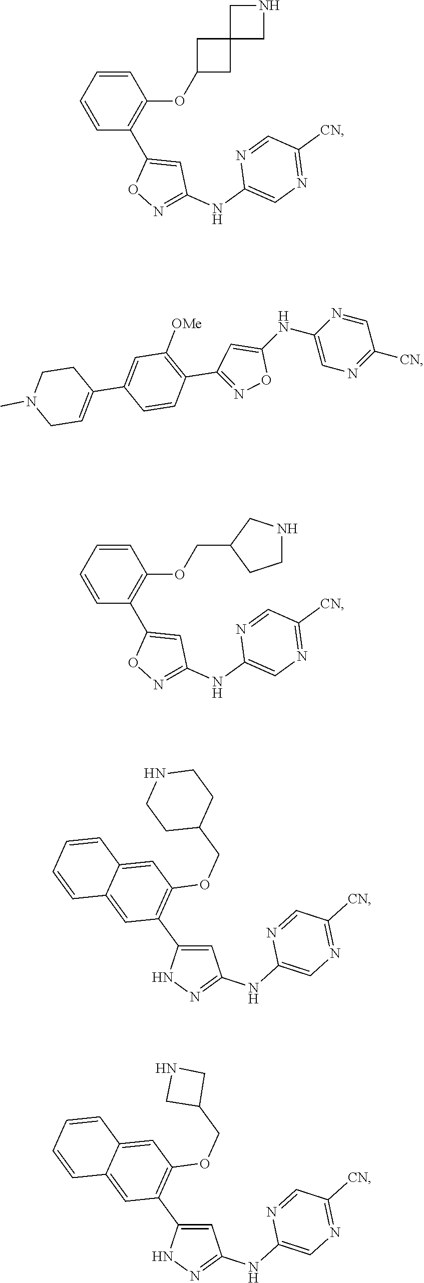 [SMILES: N#Cc1cnc(Nc2cc(-c3ccccc3OC3CC4(CNC4)C3)on2)cn1 ; COc1cc(C2=CCN(C)CC2)ccc1-c1cc(Nc2cnc(C#N)cn2)on1 ; N#Cc1cnc(Nc2cc(-c3ccccc3OCC3CCNC3)on2)cn1 ; N#Cc1cnc(Nc2cc(-c3cc4ccccc4cc3OCC3CCNCC3)[nH]n2)cn1 ; N#Cc1cnc(Nc2cc(-c3cc4ccccc4cc3OCC3CNC3)[nH]n2)cn1] 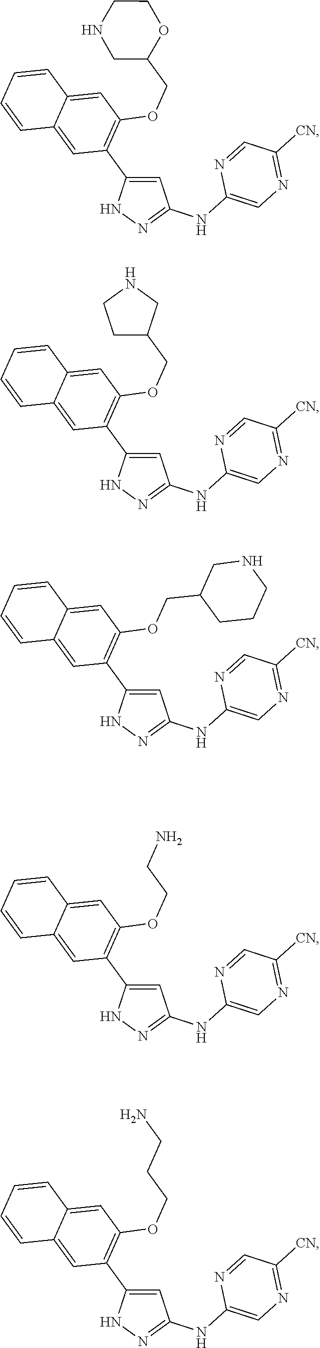 [SMILES: N#Cc1cnc(Nc2cc(-c3cc4ccccc4cc3OCC3CNCCO3)[nH]n2)cn1 ; N#Cc1cnc(Nc2cc(-c3cc4ccccc4cc3OCC3CCNC3)[nH]n2)cn1 ; N#Cc1cnc(Nc2cc(-c3cc4ccccc4cc3OCC3CCCNC3)[nH]n2)cn1 ; N#Cc1cnc(Nc2cc(-c3cc4ccccc4cc3OCCN)[nH]n2)cn1 ; N#Cc1cnc(Nc2cc(-c3cc4ccccc4cc3OCCCN)[nH]n2)cn1]
or a pharmaceutically acceptable salt thereof.

14. The compound of anyone of claims 1 to 13, use for manufacture of a medicament for treatment of a disorder associated with Checkpoint kinase-1 (CHK-1) biological pathway.

15. A method of treating disease associated with Checkpoint kinase-1 (CHK-1) in an individual in need thereof comprising administering to the individual a therapeutically effective amount of a compound of anyone of claims 1 to 13.

16. A method of treating cancer in an individual in need thereof comprising administering to the individual a therapeutically effective amount of a compound of anyone of claims 1 to 13.

17. A method of inhibiting Checkpoint kinase-1 (CHK-1) in an individual in need thereof comprising administering to the individual a therapeutically effective amount of a compound of anyone of claims 1 to 13.

18. A method of treating Idiopathic Pulmonary Fibrosis (IPF) in an individual in need thereof comprising administering to the individual a therapeutically effective amount of a compound of anyone of claims 1 to 13.

19. A method of treating Pulmonary Arterial Hypertension (PAH) in an individual in need thereof comprising administering to the individual a therapeutically effective amount of a compound of anyone of claims 1 to 13.

20. A method of treating cancer, IPF or PAH in an individual in need thereof comprising administering to the individual a therapeutically effective amount of a compound of anyone of claims 1 to 13 in combination with another therapeutic agent.

21. A pharmaceutical composition comprising a compound of anyone of claims 1 to 13 and a pharmaceutically acceptable carrier.

22. A method of treating disease associated with Checkpoint kinase-1 (CHK-1) in an individual in need thereof comprising administering to the individual the pharmaceutical composition of claim 21.

23. Use of a compound of anyone of claims 1 to 13, or a pharmaceutically acceptable salt thereof, in the manufacture of a medicament for treatment of a disease mediated by Checkpoint kinase-1 (CHK-1).

24. A kit comprising a compound of anyone of claims 1 to 13 or a pharmaceutically acceptable salt thereof.
